(19) United States
(12) Patent Application Publication
(10) Pub. No.: US 2025/0082761 A1
(43) Pub. Date: Mar. 13, 2025

(54) DUAL-TARGETED RNA POLYMERASE INHIBITORS: CONJUGATES OF BENZOXAZINO- AND SPIRO-RIFAMYCINS WITH N ALPHA-AROYL-N-ARYL-PHENYLALANINAMIDES

(71) Applicant: RUTGERS, THE STATE UNIVERSITY OF NEW JERSEY, New Brunswick, NJ (US)

(72) Inventors: Richard H. EBRIGHT, New Brunswick, NJ (US); Yon W. EBRIGHT, New Brunswick, NJ (US); Chih-Tsung LIN, New Brunswick, NJ (US)

(73) Assignee: RUTGERS, THE STATE UNIVERSITY OF NEW JERSEY, New Brunswick, NJ (US)

(21) Appl. No.: 18/683,583

(22) PCT Filed: Aug. 19, 2022
(86) PCT No.: PCT/US2022/040964

§ 371 Date: Feb. 14, 2024

Related U.S. Application Data
(60) Provisional application No. 63/235,616, filed on Aug. 20, 2021.

(51) Int. Cl. A61K 47/55; A61P 31/06

(52) U.S. Cl. CPC A61K 47/55; A61P 31/06

(57) ABSTRACT

The invention provides dual-targeted inhibitors of bacterial RNA polymerase having the general structural formula (I): wherein a is a benzoxazino-rifamycin or a spiro-rifamycin: y is a moiety that binds to the bridge-helix N-terminus target of a bacterial RNA polymerase; and P is a bond, two bonds, or a linker. The invention also provides compositions comprising such compounds, methods of making such compounds, and methods of using said compounds. The invention has applications in control of bacterial gene expression, control of bacterial growth, antibacterial chemistry, and antibacterial therapy.

### PRIORITY

This application claims priority from U.S. Provisional Patent Application No. 63/235,616, filed Aug. 20, 2021. The entire contents of this United States Provisional Patent Application is incorporated herein by reference.

### GOVERNMENT SUPPORT

The invention described herein was made with United States Government support under Grant Numbers AI1427313 and HL150852 awarded by the National Institutes of Health. The United States Government has certain rights in the invention.

### BACKGROUND

Bacterial infections remain among the most common and deadly causes of human disease. Infectious diseases are the third leading cause of death in the United States and the leading cause of death worldwide (Binder et al. (1999) Science 284, 1311-1313). Multi-drug-resistant bacteria now cause infections that pose a grave and growing threat to public health. It has been shown that bacterial pathogens can acquire resistance to first-line and even second-line antibiotics (Stuart B. Levy, The Challenge of Antibiotic Resistance, in Scientific American, 46-53 (March 1998); Walsh, C. (2000) Nature 406, 775-781; Schluger, N. (2000) Int. J. Tuberculosis Lung Disease 4, S71-S75; Raviglione et al., (2001) Ann. NY Acad. Sci. 953, 88-97). New approaches to drug development are necessary to combat the ever-increasing number of antibiotic-resistant pathogens.

RNA polymerase (RNAP) is the molecular machine responsible for transcription and is the target, directly or indirectly, of most regulation of gene expression (Ebright, R. (2000) J. Mol. Biol. 304, 687-698; Darst, S. (2001) Curr. Opin. Structl. Biol. 11, 155-162; Murakami, K. and Darst, S. (2003) Curr. Opin. Structl. Biol. 13, 31-39; Borukhov, S. and Nudler, E. (2003) Curr. Opin. Microbiol. 6, 93-100; Werner, F. (2007) Mol. Microbiol. 65, 1395-1404; Hirata, A. and Murakami, K. (2009) Curr. Opin. Structl. Biol. 19, 724-731; Jun, S., Reichlen, M., Tajiri, M. and Murakami, K. (2011) Crit. Rev. Biochem. Mol. Biol. 46, 27-40; Cramer, P. (2002) Curr. Opin. Struct. Biol. 12, 89-97; Cramer, P. (2004) Curr. Opin. Genet. Dev. 14, 218-226; Hahn, S. (2004) Nature Struct. Mol. Biol. 11, 394-403; Kornberg, R. (2007) Proc. Natl. Acad. Sci. USA 104, 12955-12961; Cramer, P., Armache, K., Baumli, S., Benkert, S., Brueckner, F., Buchen, C., Damsma, G., Dengl, S., Geiger, S., Jasiak, A., Jawhari, A., Jennebach, S., Kamenski, T., Kettenberger, Kuhn, C., Lehmann, E., Leike, K., Sydow, J. and Vannini, A. (2008) Annu. Rev. Biophys. 37, 337-352; Lane, W. and Darst, S. (2010) J. Mol. Biol. 395, 671-685; Lane, W and Darst, S. (2010) J. Mol. Biol. 395, 686-704; Werner, F. and Grohmann, D. (2011) Nature Rev. Microbiol. 9, 85-98; Vannini, A. and Cramer, P. (2012) Mol. Cell 45, 439-446). Bacterial RNAP core enzyme has a molecular mass of ˜380,000 Da and consists of one β′ subunit, one β subunit, two a subunits, and one ω subunit; bacterial RNAP holoenzyme has a molecular mass of ˜450,000 Da and consists of bacterial RNAP core enzyme in complex with the transcription initiation factor σ (Ebright, R. (2000) J. Mol. Biol. 304, 687-698; Darst, S. (2001) Curr. Opin. Structl. Biol. 11, 155-162; Cramer, P. (2002) Curr. Opin. Structl. Biol. 12, 89-97; Murakami and Darst (2003) Curr. Opin. Structl. Biol. 13, 31-39; Borukhov and Nudler (2003) Curr. Opin. Microbiol. 6, 93-100). Bacterial RNAP core subunit sequences are conserved across Gram-positive and Gram-negative bacterial species (Ebright, R. (2000) J. Mol. Biol. 304, 687-698; Darst, S. (2001) Curr. Opin. Structl. Biol. 11, 155-162; Lane, W. and Darst, S. (2010) J. Mol. Biol. 395, 671-685; Lane, W. and Darst, S. (2010) J. Mol. Biol. 395, 686-704). Eukaryotic RNAP I, RNAP II, and RNAP III contain counterparts of all bacterial RNAP core subunits, but eukaryotic-subunit sequences and bacterial-subunit sequences exhibit only limited conservation (Ebright, R. (2000) J. Mol. Biol. 304, 687-698; Darst, S. (2001) Curr. Opin. Structl. Biol. 11, 155-162; Cramer, P. (2002) Curr. Opin. Structl. Biol. 12, 89-97; Cramer, P. (2004) Curr. Opin. Genet. Dev. 14, 218-226; Lane, W. and Darst, S. (2010) J. Mol. Biol. 395, 671-685; Lane, W. and Darst, S. (2010) J. Mol. Biol. 395, 686-704).

Crystal structures have been determined for bacterial RNAP and eukaryotic RNAP II (Zhang et al., (1999) (ell 98, 811-824; Cramer et al., (2000) Science 288, 640-649; Cramer et al., (2001) Science 292, 1863-1876).

Structures also have been determined for RNAP complexes with nucleic acids, nucleotides and inhibitors (Campbell, et al. (2001) Cell 104, 901-912; Artsimovitch, et al. (2005) Cell 122, 351-363; Campbell, et al. (2005) EMBOJ. 24, 674-682; Tuske, et al. (2005) Cell 122, 541-522; Temiaov, et al. (2005) Mol. Cell 19, 655-666; Mukhopadhyay, J., Das, K., Ismail, S., Koppstein, D., Jang, M., Hudson, B., Sarafianos, S., Tuske, S., Patel, J., Jansen, R., Irschik, H., Arnold, E., and Ebright, R. (2008) Cell 135, 295-307; Belogurov, G., Vassylyeva, M., Sevostyanova, A., Appleman, J., Xiang, A., Lira, R., Webber, S., Klyuyev, S., Nudler, E., Artsimovitch, I., and Vassylyev, D. (2009) Nature. 45, 332-335; Vassylyev, D., Vassylyeva, M., Perederina, A., Tahirov, T. and Artsimovitch, I. (2007) Nature 448, 157-162; Vassylyev, D., Vassylyeva, M., Zhang, J., Palangat, M., Artsimovitch, I. and Landick, R. (2007) Nature 448, 163-168; Gnatt, et al. (2001) Science 292, 1876-1882; Westover, et al. (2004a) Science 303, 1014-1016; Westover, et al. (2004b) Cell 119, 481-489; Ketenberger, et al. (2004) Mol. Cell 16, 955-965; Bushnell, et al. (2002) Proc. Natl. Acad. Sci. U.S.A. 99, 1218-1222; Kettenberger, et al. (2005) Natl. Structl. Mol. Biol. 13, 44-48; Ho, M., Hudson, B., Das, K., Arnold, E. and Ebright, R. (2009) Curr. Opin. Structl. Biol. 19, 715-723).

Bacterial RNAP is a proven target for antibacterial therapy (Darst, S. (2004) Trends Biochem. Sci. 29, 159-162; Chopra, I. (2007) Curr. Opin. Investig. Drugs 8, 600-607; Villain-Guillot, P., Bastide, L., Gualtieri, M. and Leonetti, J. (2007) Drug Discov. Today 12, 200-208; Mariani, R. and Maffioli, S. (2009) Curr. Med. Chem. 16, 430-454; Ho, M., Hudson, B., Das, K., Arnold, E. and Ebright, R. (2009) Curr. Opin. Structl. Biol. 19, 715-723; Srivastava, A., Talaue, M., Liu, S., Degen, D, Ebright, R. Y., Sineva, E., Chakraborty, A., Druzhinin, S., Chatterjee, S., Mukhopadhyay, J., Ebright, Y., Zozula, A., Shen, J., Sengupta, S., Niedfeldt, R., Xin, C., Kaneko, T., Irschik, H., Jansen, R., Donadio, S., Connell, N. and Ebright, R. H. (2011) Curr. Opin. Microbiol. 14, 532-543). The suitability of bacterial RNAP as a target for antibacterial therapy follows from the fact that bacterial RNAP is an essential enzyme (permitting efficacy), the fact that bacterial RNAP subunit sequences are conserved (providing a basis for broad-spectrum activity), and the fact that bacterial RNAP subunit sequences are only weakly conserved in eukaryotic RNAP I, RNAP II, and RNAP III (providing a basis for therapeutic selectivity).

The rifamycin antibacterial agents—notably rifampin, rifapentine, and rifabutin—function by binding to and inhibiting bacterial RNAP (Darst, S. (2004) Trends Biochem. Sci. 29, 159-162; Ho, M., Hudson, B., Das, K., Arnold, E. and Ebright, R. (2009) Curr. Opin. Structl. Biol. 19, 715-723; Floss and Yu (2005) Chem. Rev. 105, 621-632; Campbell, et al. (2001) Cell 104, 901-912; Artsimovitch, et al. (2005) Cell 122, 351-363; Feklistov, A., Mekler, V., Jiang, Q., Westblade, L., Irschik, H., Jansen, R., Mustaev, A., Darst, S., and Ebright, R. (2008) Proc. Natl. Acad. Sci. USA 105, 14820-14825). The rifamycins bind to a site on bacterial RNAP located adjacent to the RNAP active center (“Rif target”) and prevent the extension of RNA chains beyond a length of 2-3 nt.

The rifamycins are in current clinical use in treatment of Gram-positive and Gram-negative bacterial infections (Darst, S. (2004) Trends Biochem. Sci. 29, 159-162; Ho, M., Hudson, B., Das, K., Arnold, E. and Ebright, R. (2009) Curr. Opin. Structl. Biol. 19, 715-723; Floss and Yu (2005) Chem. Rev. 105, 621-632; Campbell, et al. (2001) Cell 104, 901-912). The rifamycins are first-line treatments for tuberculosis and are the only current first-line treatments for tuberculosis able to kill non-replicating tuberculosis bacteria, to clear infection, and to prevent relapse (Mitchison, D. (2000) Int. J. Tuberc. Lung Dis. 4, 796-806). The rifamycins also are first-line treatments for biofilm-associated infections of catheters and implanted medical devices and are among the very few current antibacterial drugs able to kill non-replicating biofilm-associated bacteria (Obst, G., Gagnon, R. F., Prentis, J. and Richards, G. K. (1988) ASAIO Trans. 34, 782-784; Obst, G., Gagnon, R. F., Harris, A., Prentis, J. and Richards, G. K. (1989) Am. J. Nephrol. 9, 414-420; Villain-Guillot, P., Gualtieri, M., Bastide, L. and Leonetti, J. P. (2007) Antimicrob. Agents Chemother. 51, 3117-3121.

The clinical utility of the rifamycin antibacterial agents is threatened by the emergence and spread of bacterial strains resistant to known rifamycins (Darst, S. (2004) Trends Biochem. Sci. 29, 159-162; Ho, M., Hudson, B., Das, K, Arnold, E. and Ebright, R. (2009) Curr. Opin. Structl. Biol. 19, 715-723; Floss and Yu (2005) Chem. Rev. 105, 621-632; Campbell, et al. (2001) Cell 104, 901-912). Resistance to rifamycins typically involves substitution of residues in or immediately adjacent to the rifamycin binding site on bacterial RNAP (referred to as the “Rif target” of a bacterial RNAP)—i.e., substitutions that directly decrease binding of rifamycins. A significant and increasing percentage of cases of tuberculosis are resistant to rifampicin (1.4% of new cases, 8.7% of previously treated cases, and 100% of cases designated multidrug-resistant, in 1999-2002; Schluger, N. (2000) Int. J. Tuberc. Lung Dis. 4, S71-S75; Raviglione, et al. (2001) Ann. N.Y. Acad. Sci. 953, 88-97; Zumia, et al. (2001) Lancet Infect. Dis. 1, 199-202; Dye, et al. (2002) J. Infect. Dis. 185, 1197-1202; WHO/IUATLD (2003) Anti- tuberculosis drug resistance in the world: third global report (WHO, Geneva)). Strains of bacterial bioweapons agents resistant to rifampicin can be, and have been, constructed (Lebedeva, et al. (1991) Antibiot. Khimioter. 36, 19-22; Pomerantsev, et al. (1993) Antibiot. Khimioter. 38, 34-38; Volger, et al. (2002) Antimicrob. Agents Chemother. 46, 511-513; Marianelli, et al. (204) J. Clin. Microbiol. 42, 5439-5443).

In view of the public-health threat posed by rifamycin-resistant bacterial infections, there is an urgent need for new antibacterial agents that target bacterial RNAP and an especially urgent need for new antibacterial agents that target bacterial RNAP derivatives resistant to known rifamycins. (See Darst, S. (2004) Trends Biochem. Sci. 29, 159-162; Chopra, I. (2007) Curr. Opin. Investig. Drugs 8, 600-607; Villain-Guillot, P., Bastide, L., Gualtieri, M. and Leonetti, J. (2007) Drug Discov. Today 12, 200-208; Mariani, R. and Maffioli, S. (2009) Curr. Med. Chem. 16, 430-454; Ho, M., Hudson, B., Das, K., Arnold, E. and Ebright, R. (2009) Curr. Opin. Structl. Biol. 19, 715-723; Srivastava, A., Talaue, M., Liu, S., Degen, D., Ebright, R. Y., Sineva, E., Chakraborty, A., Druzhinin, S., Chatterjee, S., Mukhopadhyay, J., Ebright, Y., Zozula, A., Shen, J., Sengupta, S., Niedfeldt, R., Xin, C., Kaneko, T., Irschik, H., Jansen, R., Donadio, S., Connell, N. and Ebright, R.H. (2011) Curr. Opin. Microbiol. 14, 532-543; Lin, W., Mandal, S., Degen, D., Liu, Y . . . , Ebright, Y., Li, S., Feng, Y., Zhang, Y., Mandal, S., Jiang, Y., Liu, S., Gigliotti, M., Talaue, M., Connell, N., Das, K., Arnold, E., Ebright, R. (2017) Structural basis of Mycobacterium tuberculosis transcription and transcription inhibition. Mol. Cell 166, 169-179.)

Na-aroyl-N-aryl-phenylalaninamides (AAPs) inhibit bacterial RNAP, particularly Mycobacterial RNAP, through a binding site (the RNAP bridge-helix N-terminus) and a mechanism (allosteric interference with RNAP active-center conformatoinl changes required for nucleotdie addition) that differ from the binding site and mechanism of rifamycins (Lin, W., Mandal, S., Degen, D., Liu, Y . . . , Ebright, Y., Li, S., Feng, Y., Zhang, Y., Mandal, S., Jiang, Y., Liu, S., Gigliotti, M., Talaue, M., Connell, N., Das, K., Arnold, E., Ebright, R. (2017) Structural basis of Mycobacterium tuberculosis transcription and transcription inhibition. Mol. Cell 166, 169-179; Ebright, R., Ebright, Y., Mandal, S., Wilde, R., and Li, S. (2018) Antibacterial agents: N (alpha)-aroyl-N-aryl-phenylalaninamides. U.S. Pat. No. 9,919,998). Because AAPs inhibit=bacterial RNAP through a different binding site and different mechanism than rifamycins, AAPs exhibit no cross-resistance with rifamycins Structural basis of Mycobacterium tuberculosis transcription and transcription inhibition. Mol. Cell 166, 169-179; Ebright, R., Ebright, Y., Mandal, S., Wilde, R., and Li, S. (2018) Antibacterial agents: N (alpha)-aroyl-N-aryl-phenylalaninamides. U.S. Pat. No. 9,919,998). However, resistance to AAPs occurs. Resistance to AAPs involves substitution of residues in or immediately adjacent to the AAP boinding site on bacterial RNAP (the RNAP bridge-helix N-terminus)—i.e., substitutions that directly decrease binding of AAPs.

### SUMMARY

Applicant has identified compounds that inhibit bacterial RNA polymerase (RNAP) and inhibit bacterial growth. Accordingly, in one embodiment the invention provides a compound of the invention, which is a compound of formula (I):

α-β-γ (1)

or a salt thereof, wherein:
α is a benzoxazino-rifamycin or a spiro-rifamycin;
β is a bond, or two bonds, or a linker comprising at least one atom and at least two bonds; and
γ is a moiety that binds to the bridge-helix N-terminus target of a bacterial RNA polymerase.

The invention also provides a method for making a compound of formula I, wherein the compound is prepared from precursors α-β′ and ‘β-γ, where β’ and ′β are moieties that can react to form β.

The invention also provides a use of a compound of the invention to bind to a bacterial RNAP.

The invention also provides a use of a compound of the invention to inhibit a bacterial RNAP.

The invention also provides a use of a compound of the invention to inhibit bacterial gene expression.

The invention also provides a use of a compound of the invention to inhibit bacterial growth.

The invention also provides a use of a compound of the invention to inhibit a bacterial infection.

The invention also provides a composition comprising a compound of the invention or a pharmaceutically acceptable salt thereof and a pharmaceutically acceptable vehicle.

The invention also provides a method for inhibiting the growth of bacteria comprising contacting the bacteria with a compound of the invention or a salt thereof.

The invention also provides a method for inhibiting a bacterial RNAP comprising contacting the bacterial RNAP with a compound of the invention or a salt thereof.

The invention also provides a method for treating a bacterial infection in a mammal, e.g., a human, comprising administering to the mammal an effective amount of a compound of the invention or a pharmaceutically acceptable salt thereof.

The invention also provides a compound of the invention or a pharmaceutically acceptable salt thereof for use in the prophylactic or therapeutic treatment of a bacterial infection.

The invention also provides the use of a compound of the invention or a pharmaceutically acceptable salt thereof for the preparation of a medicament for treating a bacterial infection in a mammal, e.g., a human.

The invention also provides a compound of the invention or a pharmaceutically acceptable salt thereof for use in medical treatment.

The invention provides a new class of inhibitors of bacterial RNAP.

Compounds of this invention consist of a first moiety, «, that inhibits a bacterial RNAP by binding to the RNAP Rif pocket linked to a second moiety, γ, that inhibits a bacterial RNAP by binding to the RNAP brudge-helix N-terminus.

Compounds of the invention that can inhibit bacterial RNAP through two different binding sites on RNAP (i.e., the Rif target and the bridge-helix N-terminus) and two different mechanisms (i.e., the steric-occlusion mechanism of inhibitors that function through the Rif target and the allosteric mechanism of inhibitors that function through the bridge-helix N-terminus).

As a result, compounds of the invention can overcome resistance mutations that alter the the Rif target (by continuing to inhibit RNAP through the bridge-helix N-terminus) and can overcome resistance mutations that alter the bridge-helix N-terminus (by continuing to inhibit RNAP through the Rif target).

As a further result, compounds of the invention can exhibit lower resistance emergence than inhibitors that function through one of the Rif target and the bridge-helix N-terminus rifamycins and AAPs. Resistance to compounds of the invention can require a double mutational hit that alters both the Rif target and the bridge-helix N-terminus, in contrast to resistance to inhibitors that function through the Rif target or through the bridge-helix N-terminus, each of which requires only a single mutational hit that alters the Rif target or the bridge-helix N-terminus.

The invention provides compounds that can bind to a bacterial RNAP with a 2:1 stoichiometry, with a moiety of a first molecule of the compound interacting with the Rif tarfet site on RNAP, and the γ moiety of a second molecule of the compound interacting with the inhibitors that function through one of the Rif target and the bridge-helix N-terminus,

Certain compounds of the invention consist of a rifamycin RNAP inhibitor (an entity that inhibits a bacterial RNAP by binding to the RNAP Rif pocket) linked to an Na-aroyl-N-aryl-phenylalaninamide (AAP; an entity that inhibits bacterial RNAP by binding to the RNAP bridge-helix N-terminus).

Certain compounds of the invention can inhibit bacterial RNAP through two different binding sites (i.e., the rifamycin binding site and RNAP bridge-helix N-terminus) and two different mechanisms (i.e., the rifamycin mechanism and the AAP mechanisms).

As a result, certaio compounds of the invention can overcome rifamycin-resistance (by inhibiting RNAP through the AAP binding site and mechanism) and can overcome to AAP-resistance (by inhibiting RNAP through the rifamycin binding site and mechanism).

As a further result, certain compounds of the invention can exhibit lower resistance emergence than rifamycins and AAPs. Resistance to certain compounds of the invention requires a double mutational hit that alters both the rifamycin binding site and the AAP binding site, in contrast to resistance to rifamcins or AAPs, each of which requires only a single mutational hit that alters the rifamycin binding site or the AAP binding site.

The invention provides compounds that can bind to a bacterial RNAP with a 2:1 stoichiometry, with rifamycin moiety of a first molecule of the compound interacting with the rifamycin binding site on RNAP, and the AAP moiety of a second molecule of the compound interacting with the AAP binding site on RNAP

Importantly, the invention provides compounds that can exhibit potencies higher than those of known inhibitors.

Especially importantly, the invention provides compounds that can inhibit bacterial RNAP derivatives resistant to known inhibitors.

The invention provides new compositions of matter that inhibit a bacterial RNA polymerase and inhibit bacterial growth. The compounds are anticipated to have applications in analysis of RNAP structure and function, control of bacterial gene expression, control of bacterial growth, antibacterial prophylaxis, antibacterial therapy, and drug discovery.

Certain compounds of this invention inhibit a bacterial RNAP and inhibit growth of bacteria more potently than a rifamycin or an AAP.

Certain compounds of this invention may inhibit a rifamycin-resistant bacterial RNAP and inhibit growth of rifamycin-resistant bacteria much more potently than a rifamycin.

Certain compounds of this invention inhibit an AAP-resistant bacterial RNAP and inhibit growth of rifamycin-resistant bacteria much more potently than an AAP.

Compounds of this invention have particularly potent effects against drug-susceptible and drug-resistant RNAP from Mycobacteria, including Mycobacterium tuberculosis, Mycobacterium avium, and Mycobacterium abscessus.

Certain compounds of this invention have particularly potent effects against growth of drug-sensitive and drug-resistant Mycobacteria, including Mycobacterium tuberculosis, Mycobacterium avium, and Mycobacterium abscessus.

In contrast to rifamycins, which potently induce cytochrome P450 3A4 (Cyp 3A4), certain compounds of this invention do not potently induce Cyp 3A4. As a result, in contrast to rifamycins, which exhibit unfavorable drug-drug interactions due to induction of Cyp 3A4, certain compounds of this invention will not exhibit unfavorable drug-drug interactions due to induction of Cyp 3A4.

The invention provides bipartite, dual-targeted inhibitors of bacterial RNAP that contain: (i) a first moiety α; (ii) a second moiety, γ, that binds to the bridge-helix N-terminus target of a bacterial RNAP; and (iii) a linker β connecting said first and second moieties.

The invention provides bipartite, dual-targeted inhibitors that interact with bacterial RNAP through alternative interactions of α and γ. The ability of the bipartite inhibitors to interact with a bacterial RNAP alternatively through two moieties, α or γ, can result in simultaneous interactions of two molecules of bipartite inhibitor with RNAP, conferring an additive or super-additive inhibitory effects. The ability of the bipartite inhibitors to interact with RNAP alternatively through two moieties, α and γ, also can confer an ability to interact with a bacterial RNAP derivative resistant to α or γ.

The bipartite, dual-targeted inhibitors have applications in control of bacterial gene expression, control of bacterial growth, antibacterial chemistry, and antibacterial therapy.

The invention also provides intermediates and processes useful for preparing compounds of the invention.

The invention provides a method for preparing a compound that contains: (i) a first moiety α; (ii) a second moiety, γ, that binds to the bridge-helix N-terminus target of a bacterial RNAP; and (iii) a linker, β, connecting said first and second moieties. The method includes providing precursors α-β′ and ′β-γ, and reacting moieties β′ and ′β to form β. For example, one precursor may contain an aldehyde, a ketone, a protected aldehyde, or a protected ketone, and the other precursor contain a hydrazide or an amine. One precursor may contain an activated ester, an imidazolide, or an anhydride, and the other precursor contain an amine. One precursor may contain a halogen, and the other precursor contain an amine. One precursor may contain a halogen, and the other precursor contain a sulfhydryl. One precursor may contain an azide and the other precursor contain an alkyne. One precursor may contain an azide, and the other precursor contain a phosphine. One precursor may contain a boronic acid, and the other precursor contain a substituted phenol. One precursor may contain a phenylboronic acid, and the other precursor contain salicylhydroxamic acid.

These and other aspects of the present invention will be better appreciated by reference to the following drawings and Detailed Description.

### BRIEF DESCRIPTION OF THE DRAWINGS

FIG. 1 shows a sequence alignment defining the Rif target of bacterial RNAP. The sequence alignment shows amino acid residues 146, 148, 507-509, 511-513, 516, 518, 522-523, 525-526, 529, 531-534, 568, 572, 574, and 687 of the β subunit of RNAP from Escherichia coli; and corresponding residues of the β subunits of Haemophilus influenzae, Vibrio cholerae, Pseudomonas aeruginosa, Treponema pallidum, Borrelia burgdorferi, Xylella fastidiosa, Campylobacter jejuni, Neisseria meningitides, Rickettsia prowazekii, Thermotoga maritime, Chlamydia trachomatis, Mycoplasma pneumoniae, Bacillus subtilis, Staphylococcus aureus, Mycobacterium tuberculosis, Synechocystis sp., Aquifex aeolicus, Deinococcus radiodurans, Thermus thermophilus, and Thermus aquaticus (collectively, the “Rif target”); and corresponding residues of the second-largest subunits of human RNAP I, RNAP II and RNAP III.

FIG. 2 shows the position of the Rif target within the three-dimensional structure of bacterial RNAP (two orthogonal views). Sites of amino acid substitutions that confer rifamycin-resistance are shown as a dark gray solid surface (labelled R; Ovchinnikov, Y., Monastyrskaya, G., Gubanov, V., Lipkin, V., Sverdlov, E., Kiver, I., Bass, I., Mindlin, S., Danilevskaya, O., and Khesin, R. (1981) Mol. Gen. Genet. 184, 536-538; Ovchinnikov, Y., Monastyrskaya, G., Guriev, S., Kalinina, N., Sverdlov, E., Gragerov, A., Bass, I., Kiver, I., Moiseyeva, E., Igumnov, V., Mindlin, S., Nikiforov, V. and Khesin, R. (1983) Mol. Gen. Genet. 190, 344-348; Jin, D. J., and Gross, C. (1988) J. Mol. Biol. 202, 45-58; Severinov, K., Soushko, M., Goldfarb, A., and Nikiforov, V. (1993) J. Biol. Chem. 268, 14820-14825; Ho, M., Hudson, B., Das, K., Arnold, E. and Ebright, R. (2009) Curr. Opin. Structl. Biol. 19, 715-723). RNAP backbone atoms are shown in a Cα representation. The RNAP active-center Mg²⁺ is shown as a sphere.

FIGS. 3A-3B show a sequence alignment defining the bridge-helix N-terminus target of bacterial RNAP. The sequence alignment shows amino acid residues 550, 552, 555, 637, 640 and 642 of the β subunit (FIG. 3A) and 749, 750, 755, and 757 of the β′ subunit (FIG. 3B) of RNAP from Escherichia coli (ECOLI), and corresponding residues of the β and β′ subunits of Mycobacterium tuberculosis (MYCTU), Mycobacterium avium (MYCA1), Mycobacterium abscessus (MYCA9), Mycobacterium smegmatis (MYCSM), Salmonella typhimurium (SALTY), Klebsiella pneumoniae (KLEP7), Enterococcus cloacae (ENTCC), Vibrio cholerae (VIBCH), Haemophilus influenzae (HAEIN), Neisseria gonorrhoeae (NEIG1), Stenotrophomonas maltophilia (STPMP), Moraxella catarrhalis (MORCA), Acinetobacter baumannii (ACIBC), Pseudomonas aeruginosa (PSEAE), Staphylococcus aureus (STAAU), Staphylococcus epidermidis (STAEQ), Enterococcus faecalis (ENTFA), Streptococcus pyogenes (STRP1), Streptococcus pneumoniae (STRP2), Clostridium difficile (CDIFF), Thermus thermophilus (THETH), Thermus aquaticus (THEAQ), and Deinococcus radiodurans (DEIRA) (collectively, the “bridge-helix N-terminus target”); and corresponding residues of the second-largest subunits of human RNAP I, RNAP II, and RNAP III. Defining residues of the bridge-helix N-terminus target are boxed and are numbered at top as in Eschericia coli RNAP (in parentheses) and as in Mycobacterium tuberculosis RNAP.

FIG. 4 shows the position of the bridge-helix N-terminus within the three-dimensional structure of bacterial RNAP (two orthogonal views). Sites of amino acid substitutions that confer AAP-resistance and/or CBR-resistance are shown as a dark gray solid surface (labelled B; Artsimovitch, I., Chu, C., Lynch, A. S., and Landick, R. (2003). Science 302, 650-654; Feng, Y., Degen, D., Wang, X., Gigliotti, M., Liu, S., Zhang, Y., Das, D., Michalchuk, T., Ebright, Y. W., Talaue, M., Connell, N., and Ebright, R. H . . . (2015). Structure 23, 1470-1481; Bae, B., Nayak, D., Ray, A., Mustaev, A., Landick, R., and Darst, S. A. (2015). Proc. Natl. Acad. Sci. USA 112, E4178-E4187; Ebright, R. H., Ebright, Y., Mandal, S., Wilde, R., and Li, S. (2015) WO2015/120320; Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179). RNAP backbone atoms are shown in a Co representation. The RNAP active-center Mg2+ is shown as a sphere.

### DETAILED DESCRIPTION OF THE INVENTION

### Definitions

The following definitions are used, unless otherwise described: halo or halogen is fluoro, chloro, bromo, or iodo. Alkyl, alkoxy, etc. denote both straight and branched groups; but reference to an individual radical such as propyl embraces only the straight chain radical, a branched chain isomer such as isopropyl being specifically referred to.

The term “alkyl”, by itself or as part of another substituent, means, unless otherwise stated, a straight-chain, branched-chain, or cycle-containing-chain hydrocarbon radical, having the number of carbon atoms designated (i.e., C₁₋₆ means one to six). Examples include C₁-C₆) alkyl, (C₂-C₆) alkyl and (C₃-C₆) alkyl. Examples of alkyl groups include methyl, ethyl, propyl, isopropyl, butyl, isobutyl, sec-butyl, pentyl, isopentyl, hexyl, isohexyl, cyclopropyl, cyclobutyl, cyclopentyl, cyclohexyl, cyclopropyl-(C₁-C₃) alkyl, cyclobutyl-(C₁-C₂) alkyl, and cyclopentyl-(C₁) alkyl), and isomers and higher homologs.

The term “alkoxy” refers to an alkyl groups attached to the remainder of the molecule via an oxygen atom (“oxy”).

The term “aryl” as used herein refers to a single all carbon aromatic ring or a multiple condensed all carbon ring system wherein at least one of the rings is aromatic. For example, in certain embodiments, an aryl group has 6 to 20 carbon atoms, 6 to 14 carbon atoms, 6 to 12 carbon atoms, or 6 to 10 carbon atoms. Aryl includes a phenyl radical. Aryl also includes multiple condensed carbon ring systems (e.g., ring systems comprising 2, 3 or 4 rings) having about 9 to 20 carbon atoms in which at least one ring is aromatic and wherein the other rings may be aromatic or not aromatic (i.e., cycloalkyl. The rings of the multiple condensed ring system can be connected to each other via fused, spiro and bridged bonds when allowed by valency requirements. It is to be understood that the point of attachment of a multiple condensed ring system, as defined above, can be at any position of the ring system including an aromatic or a carbocycle portion of the ring. Non-limiting examples of aryl groups include, but are not limited to, phenyl, indenyl, indanyl, naphthyl, 1, 2, 3, 4-tetrahydronaphthyl, anthracenyl, and the like.

The term “heteroaryl” as used herein refers to a single aromatic ring that has at least one atom other than carbon in the ring, wherein the atom is selected from the group consisting of oxygen, nitrogen and sulfur; “heteroaryl” also includes multiple condensed ring systems that have at least one such aromatic ring. Thus, “heteroaryl” includes single aromatic rings of from about 1 to 6 carbon atoms and about 1-4 heteroatoms selected from the group consisting of oxygen, nitrogen and sulfur. The sulfur and nitrogen atoms may also be present in an oxidized form provided the ring is aromatic. Exemplary heteroaryl ring systems include but are not limited to pyridyl, pyrimidinyl, oxazolyl or furyl. In one embodiment, the heteroaryl is a (C₃-C₅) heteroaryl

The term “acyl” means-C(═O)R, wherein R is (C₁-C₆) alkyl. In one embodiment, “acyl” is —C(═O)CH₃.

The term “aroyl” means-C(═O)R, wherein R is aryl. In one embodiment, aroyl is —(C(═O)-phenyl.

The term “heteroaroyl” means-C(═O)R, wherein R is heteroaryl. In one embodiment, heteroaroyl is —(C(═O)—(C₃-C₅) heteroaryl.

The term “amine” means —NRR, wherein each R is one of H and (C₁-C₆) alkyl.

The term “amide” means —C(═O) NRR, wherein each R is one of H and (C₁-C₆) alkyl.

The term “carbamidyl” means —NHC(═O)R, wherein R is (C₁-C₆) alkyl.

The term “ester” means —C(═O)O(C₁-C₆)alkyl.

The term, “hydroxyl” means —OH.

The term “phosphate” means —OP(═O)(OH)₂.

The term “O-methylphosphate” means —OP(═O)(OR)₂ m wherein each R independently is one of H and methyl.

The term “alkoxy-substituted alkyl” means a (C₁-C₆) alkyl group that is substituted with one or more (e.g., 1, 2, or 3)(C₁-C₆) alkoxy groups.

The term “amino-substituted alkyl” means a (C₁-C₆) alkyl group that is substituted with one or more (e.g., 1, 2, or 3) amine (—NRR) groups.

The term “aryl-substituted alkyl” means a (C₁-C₆) alkyl group that is substituted with one or more (e.g., 1, 2, or 3) aryl groups. The term includes benzyl and phenethyl.

The terms “treat”, “treatment”, or “treating” to the extent it relates to a disease or condition includes inhibiting the disease or condition, eliminating the disease or condition, and/or relieving one or more symptoms of the disease or condition. The terms “treat”, “treatment”, or “treating” also refer to both therapeutic treatment and/or prophylactic treatment or preventative measures, wherein the object is to prevent or slow down (lessen) an undesired physiological change or disorder, such as, for example, the development or spread of cancer. For example, beneficial or desired clinical results include, but are not limited to, alleviation of symptoms, diminishment of extent of disease or disorder, stabilized (i.e., not worsening) state of disease or disorder, delay or slowing of disease progression, amelioration or palliation of the disease state or disorder, and remission (whether partial or total), whether detectable or undetectable. “Treat”, “treatment”, or “treating,” can also mean prolonging survival as compared to expected survival if not receiving treatment. Those in need of treatment include those already with the disease or disorder as well as those prone to have the disease or disorder or those in which the disease or disorder is to be prevented. In one embodiment “treat”, “treatment”, or “treating” does not include preventing or prevention.

Unless otherwise specified, the term “binds” used herein refers to high-affinity specific binding (i.e., an interaction for which the equilibrium dissociation constant, Kd, is less than about 100 μM and preferably is less than about 10 μM).

Unless otherwise specified, the term “rifamycin” used herein encompasses both the napthol (reduced) and napthoquinone (oxidized) forms of a rifamycin, and both the 25-O-acetyl and 25-OH forms of a rifamycin (see Sensi, P., Maggi, N., Furesz, S. and Maffii, G. (1966) Antimicrobial Agents Chemother 6, 699-714; Rinehart, K. (1972) Accts. Chem. Res. 5, 57-64; Wehrli (1977) Topics Curr. Chem. 72, 21-49; Floss, et al. (2005) Chem. Rev. 105, 621-632; Aristoff, P., Garcia, G. A., Kirchoff, P. and Showalter, H. D. H. (2010) Tuberculosis 90, 94-118).

Unless otherwise specified, structures depicted herein are intended to include all stereochemical forms of the structure; i.e., the R and S configurations for each asymmetric center. Therefore, single stereochemical isomers, as well as enantiomeric and diastereomeric mixtures, of the present compounds are within the scope of the invention.

Unless otherwise specified, structures depicted herein are intended to include compounds which differ only in the presence of one or more isotopically enriched atoms. For example, compounds having the present structures, except for the replacement of a hydrogen atom by a deuterium atom or a tritium atom, except for the replacement of a deuterium atom by a hydrogen atom or a tritium atom, or except for the replacement of a carbon by a ¹³C- or ¹⁴C-enriched carbon atom, are within the scope of this invention.

Compounds of this invention may exist in tautomeric forms, such as keto-enol tautomers. The depiction of a single tautomer is understood to represent the compound in all of its tautomeric forms.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any non-toxic salt that, upon administration to a recipient, is capable of providing, either directly or indirectly, a compound of this invention.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para-toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para-bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne-1,4-dioate, hexyne-1,6-dioate, benzoate, chlorobenzoate, methylbenzoate, dinitrobenzoate, hydroxybenzoate, methoxybenzoate, phthalate, terephthalate, sulfonate, xylene sulfonate, phenylacetate, phenylpropionate, phenylbutyrate, citrate, lactate, β-hydroxybutyrate, glycolate, maleate, tartrate, methanesulfonate, propanesulfonate, naphthalene-1-sulfonate, naphthalene-2-sulfonate, mandelate and other salts. In one embodiment, pharmaceutically acceptable acid addition salts include those formed with mineral acids such as hydrochloric acid and hydrobromic acid, and especially those formed with organic acids such as maleic acid.

The pharmaceutically acceptable salt may also be a salt of a compound of the present invention having an acidic functional group, such as a carboxylic acid functional group, and a base. Exemplary bases include, but are not limited to, hydroxide of alkali metals including sodium, potassium, and lithium; hydroxides of alkaline earth metals such as calcium and magnesium; hydroxides of other metals, such as aluminum and zinc; ammonia, organic amines such as unsubstituted or hydroxyl-substituted mono-, di-, or tri-alkylamines, dicyclohexylamine; tributyl amine; pyridine; N-methyl, N-ethylamine; diethylamine; triethylamine; mono-, bis-, or tris-(2-OH—(C₁-C₆)-alkylamine), such as N,N-dimethyl-N-(2-hydroxyethyl) amine or tri-(2-hydroxyethyl) amine; N-methyl-D-glucamine; morpholine; thiomorpholine; piperidine; pyrrolidine; and amino acids such as arginine, lysine, and the like.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, polymorphic, or stereoisomeric form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase.

When a bond in a compound formula herein is drawn in a non-stereochemical manner (e.g. flat), the atom to which the bond is attached includes all stereochemical possibilities. When a bond in a compound formula herein is drawn in a defined stereochemical manner (e.g. bold, bold-wedge, dashed or dashed-wedge), it is to be understood that the atom to which the stereochemical bond is attached is enriched in the absolute stereoisomer depicted unless otherwise noted. In one embodiment, the compound may be at least 51% the absolute stereoisomer depicted. In another embodiment, the compound may be at least 60% the absolute stereoisomer depicted. In another embodiment, the compound may be at least 80% the absolute stereoisomer depicted. In another embodiment, the compound may be at least 90% the absolute stereoisomer depicted. In another embodiment, the compound may be at least 95 the absolute stereoisomer depicted. In another embodiment, the compound may be at least 99% the absolute stereoisomer depicted.

Specific values listed below for radicals, substituents, and ranges, are for illustration only; they do not exclude other defined values or other values within defined ranges for the radicals and substituents. It is to be understood that two or more values may be combined. It is also to be understood that the values listed herein below (or subsets thereof) can be excluded.

A specific alkyl is a (C₁-C₆) alkyl. Specifically, (C₁-C₆) alkyl can be, for example, methyl, ethyl, propyl, isopropyl, butyl, isobutyl, sec-butyl, pentyl, isopentyl, hexyl, isohexyl, cyclopropyl, cyclobutyl, cyclopentyl, cyclohexyl, cyclopropyl-(C₁-C₃) alkyl, cyclobutyl-(C₁-C₂) alkyl, and cyclopentyl-(C₁) alkyl); (C₁-C₆) alkoxy can be, for example, methoxy, ethoxy, propoxy, isopropoxy, butoxy, iso-butoxy, sec-butoxy, pentoxy, 3-pentoxy, or hexyloxy; and (C₁-C₆) alkanoyl can be, for example, acetyl, propanoyl or butanoyl; and aryl can be phenyl, indenyl, or naphthyl.

A specific alkoxy is a (C₁-C₆) alkoxy. Specifically, (C₁-C₆) alkoxy can be, for example, methoxy, ethoxy, propoxy, isopropoxy, butoxy, isobutoxy, sec-butyoxy, pentoxy, isopentoxy, hexoxy, isohexoxy, cyclopropoxy, cyclobutoxy, cyclopentoxy, cyclohexoxy, cyclopropyl-(C₁-C₃) alkoxy, cyclobutyl-(C₁-C₂) alkoxy, and cyclopentyl-(C₁) alkoxy.

### Dual-Targeted Inhibitors of RNAP

Certain embodiments of the invention provide a new class of inhibitors of RNAP that inhibit RNAP through two different tabiundiung sites and two different mechanisms. Certain embodiments of the invention provide novel inhibitors of RNAP that kill bacterial pathogens more potently than current inhibitors. For example, certain embodiments exhibit inhibition activities higher than known inhibitors. Another aspect of the invention is the provision of novel inhibitors of RNAP that kill bacterial pathogens resistant to current inhibitors.

Moiety that Binds to the Rifamycin Target of a Bacterial RNA Polymerase (α)

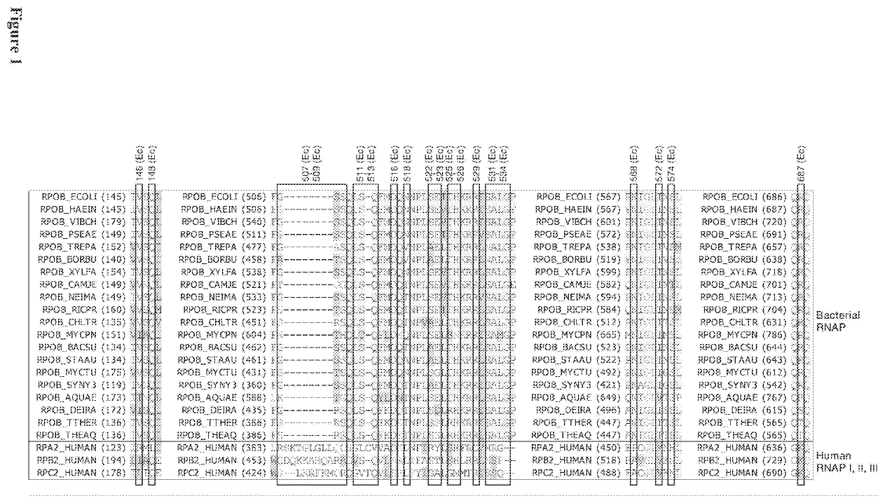

A region located within the RNAP active-center cleft—a region that comprises amino acids 146, 148, 507-509, 511-513, 516, 518, 522-523, 525-526, 529, 531-534, 568, 572, 574, and 687 of the RNAP β subunit in RNAP from Escherichia coli—is a useful target for compounds that inhibit transcription, including, by way of example, rifamycins, streptovaricins, tolypomycins, and sorangicins (Sensi, P., Maggi, N., Furesz, S. and Maffii, G. (1966) Antimicrobial Agents Chemother 6, 699-714; Rinehart (1972) Accts. Chem. Res. 5, 57-64; Wehrli (1977) Topies Curr. Chem. 72, 21-49; Campbell, et al. (2001) Cell 104, 901-912; Artsimovitch, et al. (2005) Cell 122, 351-363; Floss, et al. (2005) Chem. Rev. 105, 621-632; Aristoff, P., Garcia, G. A., Kirchoff, P. and Showalter, H. D. H. (2010) Tuberculosis 90, 94-118; Nitta, et al. (1968) J. Antibiotics 21, 521-522; Morrow, et al. (1979) J. Bacteriol. 137, 374-383; Kondo, et al. (1972) J. Antibiotics 25, 16-24; Rommelle, et al. (1990) J. Antibiotics 43, 88-91; O'Neill, et al. (2000) Antimicrobial Agents Chemother. 44, 3163-3166; Campbell, et al. (2005) EMBO J. 24, 1-9; Ho, M., Hudson, B., Das, K., Arnold, E. and Ebright, R. (2009) Curr. Opin. Structl. Biol. 19, 715-723; Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179; FIGS. 1,2). This region is referred to herein as the “Rif target,” reflecting the fact that it serves as the binding site for rifamycins, among other compounds.

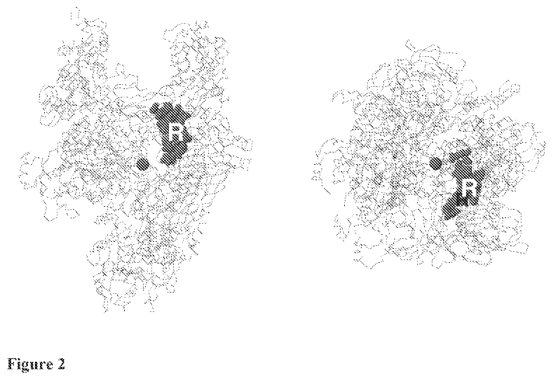

The Rif target includes residues that are invariant or nearly invariant in RNAP from bacterial species, but that are radically different in RNAP from eukaryotic species (FIG. 1). The Rif target forms a shallow pocket within the wall of the RNAP active-center cleft (FIG. 2). A compound that binds to the Rif target of a bacterial RNAP can block bacterial RNA synthesis (e.g., by sterically blocking extension of RNA chains beyond a length of 2-3 nt), can inhibit bacterial gene expression, and can inhibit bacterial growth.

The Rif target referred to above in RNAP from Escherichia coli is similar in amino acid sequence in RNAP from most or all species of bacteria (FIG. 1). For example, amino acid residues 146, 148, 507-509, 511-513, 516, 518, 522-523, 525-526, 529, 531-534, 568, 572, 574, and 687 of the β subunit of RNAP from Escherichia coli exhibit high similarity to amino acid residues 135-137, 463-465, 467-469, 472, 474, 478-479, 481-482, 485, 487-490, 524, 526, and 645 of the B subunit of RNAP from Bacillus subtilis (FIG. 1). Thus, a molecule that binds to the Rif target of, and inhibits RNA synthesis by, RNAP from Escherichia coli also is likely to bind to the Rif target of, and inhibit RNA synthesis by, RNAP from other species of bacteria.

In contrast, the Rif target differs radically in amino acid sequence between bacterial RNAP and eukaryotic RNAP, including human RNAP I, human RNAP II, and human RNAP III (FIG. 1). This allows for the identification of molecules that bind, in a Rif-target-dependent fashion, to a bacterial RNAP, but that do not bind, or that bind substantially less well, to a eukaryotic RNAP. This also allows for the identification of molecules that inhibit, in a Rif-target-dependent fashion, an activity of a bacterial RNAP, but that do not inhibit, or that inhibit substantially less well, an activity of a eukaryotic RNAP. This differentiation is important, because it permits the identification of bacterial-RNAP-selective binding molecules and bacteria-selective inhibitors.

Ligands that bind to the Rif target of, and inhibit RNA synthesis by, a bacterial RNAP are known in the art. Such ligands include, for example, rifamycins (a class of compounds that includes, for example, rifamycin SV, rifamycin S, rifamycin B, rifampin, rifapentine, rifalazil, and rifabutin), streptovaricins, tolypomycins, and sorangicins (Sensi, P., Maggi, N., Furesz, S. and Maffii, G. (1966) Antimicrobial Agents Chemother 6, 699-714; Rinehart (1972) Accts. Chem. Res. 5, 57-64; Wehrli (1977) Topics Curr. Chem. 72, 21-49; Campbell, et al. (2001) Cell 104, 901-912; Artsimovitch, et al. (2005) Cell 122, 351-363; Floss, et al. (2005) Chem. Rev. 105, 621-632; Aristoff, P., Garcia, G. A., Kirchoff, P. and Showalter, H. D. H. (2010) Tuberculosis 90, 94-118; Nitta, et al. (1968) J. Antibiotics 21, 521-522; Morrow, et al. (1979) J. Bacteriol. 137, 374-383; Kondo, et al. (1972) J. Antibiotics 25, 16-24; SOR: Rommelle, et al. (1990) J. Antibiotics 43, 88-91; O'Neill, et al. (2000) Antimicrobial Agents Chemother. 44, 3163-3166; Campbell, et al. (2005) EMBO J. 24, 1-9; Ho, M., Hudson, B., Das, K., Arnold, E. and Ebright, R. (2009) Curr. Opin. Structl. Biol. 19, 715-723; Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179). The references cited above are incorporated herein in their entirety.

Benzoxazino derivatives of rifamycins are known in the art and include, for example, rifalazil. Spiro derivatives of rifamycins are known in the art and include, for example, rifabutin.

Derivatives of rifamycins in which the C25 acetyl group is replaced by C25 hydroxyl, a C25 O-acyl group other than acetyl, a C25 O-carbamate group are known in the art and can provide potential advantages in terms of.increased solubility, incaresed activity, and/or decreased susceptibility to resistance (Combrink et al. (2007) Bioorg. Med. Chem. Lett. 17, 522-526).

Resistance to rifamycins, streptovaricins, tolypomycins, and sorangicins usually arises from mutations that result in amino acid substitutions in, or immediately adjacent to Rif target (Campbell, et al. (2001) Cell 104, 901-912; Artsimovitch, et al. (2005) Cell 122, 351-363; Floss, et al. (2005) Chem. Rev. 105, 621-632; Aristoff, P., Garcia, G. A., Kirchoff, P. and Showalter, H. D. H. (2010) Tuberculosis 90, 94-118; O'Neill, et al. (2000) Antimicrobial Agents Chemother. 44, 3163-3166; Campbell, et al. (2005) EMBO J. 24, 1-9; Ho, M., Hudson, B., Das, K., Arnold, E. and Ebright, R. (2009) Curr. Opin. Structl. Biol. 19, 715-723).

In one embodiment, α is:

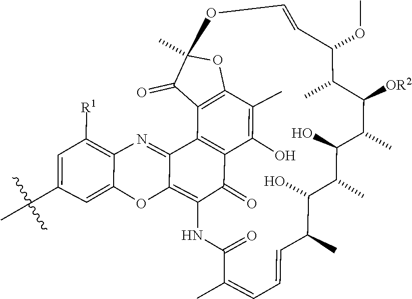

where R¹ is one of hydrogen, hydroxyl, and (C₁-C₆)alkyl, and R² is one of hydrogen, R^{x}, and —C(═O) NR^{a}R^{b}; wherein each R^{a} and R^{b} is one of hydrogen and (C₁-C₆) alkyl or R^{a} and R^{b} together with the nitrogen to which they are attached form a aziridino, azetidino, morpholino, piperazino, pyrrolidino or piperidino; and R^{x} is “(C₁-C₆) alkanoyl optionally substituted by halogen, aroyl optionally substituted by halogen, (C₃-C₅) heteroaroyl optionally substituted by halogen.

In one embodiment, α is a bednzoxazino-rifamycin.

In one embodiment, α is:

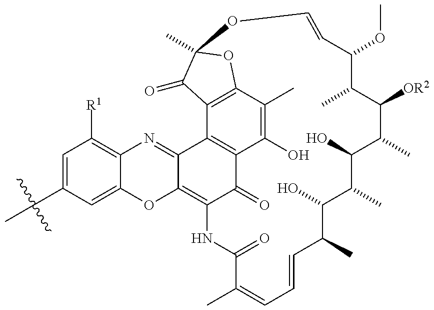

where R¹ is one of hydrogen, hydroxyl, and (C₁-C₆)alkyl, and R² is one of hydrogen, (C₁-C₆) alkanoyl, and —C(═O)NR^{a}R^{b}; wherein each R^{a} and R^{b} is one of hydrogen and (C₁-C₆) alkyl or R^{a} and R^{b} together with the nitrogen to which they are attached form a aziridino, azetidino, morpholino, piperazino, pyrrolidino or piperidino

In one embodiment, α is a spiro-rifamycin.

In one embodiment, α is:

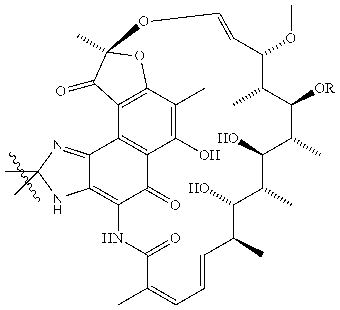

where R is one of hydrogen, R^{x}, and —C(═O)NR^{a}R^{b}; wherein each R^{a} and R^{b} is one of hydrogen and (C₁-C₆) alkyl or R^{a} and R^{b} together with the nitrogen to which they are attached form a aziridino, azetidino, morpholino, piperazino, pyrrolidino or piperidino; and R^{x} is “(C₁-C₆) alkanoyl optionally substituted by halogen, aroyl optionally substituted by halogen, (C₃-C₅) heteroaroyl optionally substituted by halogen.

In one embodiment, α is:

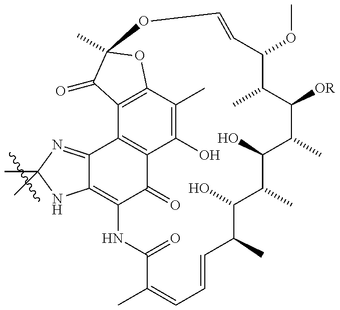

where R is one of hydrogen, (C₁-C₆) alkanoyl, and —C(═O)NR^{a}R^{b}, wherein each R^{a} and R^{b} is one of hydrogen and (C₁-C₆) alkyl or R^{a} and R^{b} together with the nitrogen to which they are attached form a aziridino, azetidino, morpholino, piperazino, pyrrolidino or piperidino.
Moiety that Binds to the Bridge-Helix N-Terminus of a Bacterial RNA Polymerase (γ)

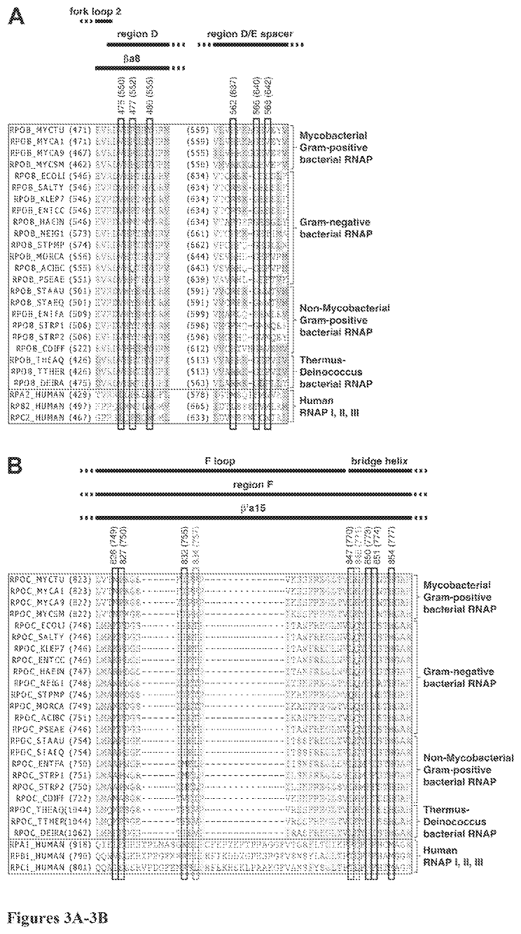

A region of RNAP that comprises amino acids 550, 552, 555, 637, 640 and 642 of the β subunit and amino acids 749, 750, 755, and 757 of the B′ subunit of RNAP from Escherichia coli is a useful target for compounds that inhibit transcription, including, by way of example, CBR hydoxamidines and pyrazoles (CBRs) and No-aroyl-N-aryl-phenylalanianmides (AAPs) (Artsimovitch, I., Chu, C., Lynch, A. S., and Landick, R. (2003). Science 302, 650-654; Feng, Y., Degen, D., Wang, X., Gigliotti, M., Liu, S., Zhang, Y., Das, D., Michalchuk, T., Ebright, Y. W., Talaue, M., Connell, N., and Ebright, R. H. (2015). Structure 23, 1470-1481; Bae, B., Nayak, D., Ray, A., Mustaev, A., Landick, R., and Darst, S. A. (2015). Proc. Natl. Acad. Sci. USA 112, E4178-E4187; Ebright, R. H., Ebright, Y., Mandal, S., Wilde, R., and Li, S. (2015) WO2015/120320; Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179; FIGS. 3,4). This region is referred to herein as the “bridge-helix N-terminus target,” reflecting the fact that it is includes residues of a structural element of RNAP referred to as the “bridge-helix N-terminus.”

The bridge-helix N-terminus target includes residues that are invariant or nearly invariant in RNAP from Gram-negative bacterial species, but that are radically different in RNAP from eukaryotic species (Feng, Y., Degen, D., Wang, X., Gigliotti, M., Liu, S., Zhang, Y., Das, D., Michalchuk, T., Ebright, Y. W., Talaue, M., Connell, N., and Ebright, R. H . . . (2015). Structure 23, 1470-1481; Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179; FIG. 3).

The bridge-helix N-terminus target also includes residues that are invariant or nearly invariant in RNAP from Mycobacterial Gram-positive bacterial species, but that are radically different in RNAP from eukaryotic species (Feng, Y., Degen, D., Wang, X., Gigliotti, M., Liu, S., Zhang, Y., Das, D., Michalchuk, T., Ebright, Y. W., Talaue, M., Connell, N., and Ebright, R. H. (2015). Structure 23, 1470-1481; Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. (ell 66, 169-179; FIG. 3).

The bridge-helix N-terminus target also includes residues that are invariant or nearly invariant in RNAP from non-Mycobacterial Gram-positive bacterial species, but that are radically different in RNAP from eukaryotic species (Feng, Y., Degen, D., Wang, X., Gigliotti, M., Liu, S., Zhang, Y., Das, D., Michalchuk, T., Ebright, Y. W., Talaue, M., Connell, N., and Ebright, R. H. (2015). Structure 23, 1470-1481; Lin, W., Mandal, S., Degen, D., Liu, Y., Ebright, Y. W., Li, S., Feng, Y., Zhang, Y., Mandal, S, Jiang, Y., Liu, S., Gigliotti, M, Talaue, M., Connell, N., Das, K., Arnold, E., and Ebright, R. H. (2017) Mol. Cell 66, 169-179; FIG. 3).

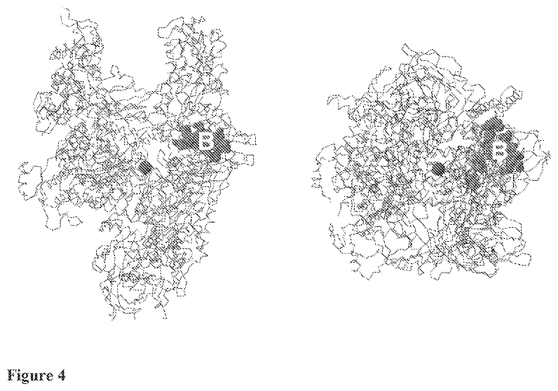

The bridge-helix N-terminus target comprises a pocket overlapping the bridge-helix N-terminus (Feng, Y., Degen, D, Wang, X., Gigliotti, M., Liu, S., Zhang, Y., Das, D., Michalchuk, T., Ebright, Y. W., Talaue, M., Connell, N., and Ebright, R. H. (2015). Structure 23, 1470-1481; Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E, and Ebright, R. H. (2017) Mol. Cell 66, 169-179; FIG. 4). A compound that binds to the bridge-helix N-terminus target target of a bacterial RNAP can block bacterial RNA synthesis (e.g., interfering with bridge-helix conformational dynamics required for RNA synthesis), can inhibit bacterial gene expression, and can inhibit bacterial growth.

The bridge-helix N-terminus target in RNAP from Escherichia coli is similar in amino acid sequence in RNAP from most or all other Gram-negative bacterial species (Feng, Y., Degen, D., Wang, X., Gigliotti, M., Liu, S., Zhang, Y., Das, D., Michalchuk, T., Ebright, Y. W., Talaue, M., Connell, N., and Ebright, R. H. (2015). Structure 23, 1470-1481; Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179; FIG. 3). For example, amino acid residues 550, 552, 555, 637, 640, and 642 of the B subunit and amino acids 749, 750, 755, and 757 of the B′ subunit of RNAP from Escherichia coli exhibit high similarity to corresponding amino acid residues of the B and B′ subunits of RNAP from other Gram-negative bacterial species (FIG. 3). Thus, a molecule that binds to the bridge-helix N-terminus target of, and inhibits RNA synthesis by, RNAP from Escherichia coli also is likely to bind to the bridge-helix N-terminus target of, and inhibit RNA synthesis by, RNAP from other Gram-negative bacterial species.

The bridge-helix N-terminus target in RNAP from Mycobacterium tuberculosis is similar in amino acid sequence in RNAP from most or all other Mycobacterial species (Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179; FIG. 3). For example, amino acid residues 475, 477, 480, 562, 566, and 568 of the β subunit and amino acids 826, 827, 832, 834, 847, 848, 850, 851, and 854 of the B′ subunit of RNAP from Mycobacterium tuberculosis exhibit high similarity to corresponding amino acid residues of the B and B′ subunits of RNAP from other Mycobacterial bacterial species (FIG. 3). Thus, the a molecule that binds to the bridge-helix N-terminus target of, and inhibits RNA synthesis by, RNAP from Mycobacterium tuberculosis also is likely to bind to the bridge-helix N-terminus target of, and inhibit RNA synthesis by, RNAP from other Mycobacterial bacterial species.

The bridge-helix N-terminus target in RNAP from Staphylococcus aureus is similar in amino acid sequence in RNAP from most or all other non-Mycobacterial Gram-positive bacterial species (Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179; FIG. 3). For example, amino acid residues 505. 5-7, 510, 594, 597, and 599 of the β subunit and amino acids 757, 758, 763, 765, 778, 779, 781, 782, and 785 of the β′ subunit of RNAP from Staphylococcus aureus exhibit high similarity to corresponding amino acid residues of the β and B′ subunits of RNAP from other non-Mycobacterial Gram-positive bacterial species (FIG. 3). Thus, the α molecule that binds to the bridge-helix N-terminus target of, and inhibits RNA synthesis by, RNAP from Staphylococcus aureus also is likely to bind to the bridge-helix N-terminus target of, and inhibit RNA synthesis by, RNAP from other non-Mycobacterial Gram-positive bacterial species.

In contrast, the bridge-helix N-terminus target differs radically in amino acid sequence between bacterial RNAP and eukaryotic RNAP, including human RNAP I, human RNAP II, and human RNAP III (FIG. 1). This allows for the identification of molecules that bind, in a bridge-helixpN-terminus-target-dependent fashion, to a bacterial RNAP, but that do not bind, or that bind substantially less well, to a eukaryotic RNAP. This also allows for the identification of molecules that inhibit, in a bridge-helixpN-terminus-target-dependent fashion, an activity of a bacterial RNAP, but that do not inhibit, or that inhibit substantially less well, an activity of a eukaryotic RNAP. This differentiation is important, because it permits the identification of bacterial-RNAP-selective binding molecules and bacteria-selective inhibitors.

Assays that enable identification of compounds that bind to the bridge-helix N-terminus, and inhibit RNA synthesis by, a bacterial RNA polymerase are known in the art [Feng, Y., Degen, D., Wang, X., Gigliotti, M., Liu, S., Zhang, Y., Das, D., Michalchuk, T., Ebright, Y. W., Talaue, M., Connell, N., and Ebright, R. H. (2015). Structure 23, 1470-1481; Bae, B., Nayak, D., Ray, A., Mustaev, A., Landick, R., and Darst, S. A. (2015). Proc. Natl. Acad. Sci. USA 112, E4178-E4187; Ebright, R. H., Ebright, Y., Mandal, S., Wilde, R., and Li, S. (2015) WO2015/120320; Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179].

Compounds that bind to the bridge-helix N-terminus target of, and inhibit RNA synthesis by, a bacterial RNAP are known in the art. Such ligands include, for example, CBR hydroxamidines and CBR pyrazoles (CBRs; Li, L., Chen, X., Fan, P., Mihalic, J. and Cutler, S. (2001) WO/2001/051456; Li, L., Chen, X., Cutler, S and Mann, J. (2001) Pyrazole antimicrobial agents. WO/2001/082930; Artsimovitch, I., Chu, C., Lynch, A. S., and Landick, R. (2003). Science 302, 650-654; Feng, Y., Degen, D., Wang, X., Gigliotti, M., Liu, S., Zhang, Y., Das, D., Michalchuk, T., Ebright, Y. W., Talaue, M., Connell, N., and Ebright, R. H. (2015). Structure 23, 1470-1481; Bae, B., Nayak, D., Ray, A., Mustaev, A., Landick, R., and Darst, S. A. (2015). Proc. Natl. Acad. Sci. USA 112, E4178-E4187) and Nα-aroyl-N-aryl-phenylalanianmides (AAPs; Ebright, R. H., Ebright, Y., Mandal, S., Wilde, R., and Li, S. (2015) WO2015/120320; Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179). The references cited above are incorporated herein in their entirety.

CBR hydroxamidines and CBR pyrazoles (CBRs) are classes of antibacterial agents known in the art that function by inhibiting RNAP through binding to the bridge-helix N-terminus target (Li, L., Chen, X., Fan, P., Mihalic, J. and Cutler, S. (2001) WO/2001/051456; Li, L., Chen, X., Cutler, S. and Mann, J. (2001) Pyrazole antimicrobial agents. WO/2001/082930; Artsimovitch, I., Chu, C., Lynch, A. S., and Landick, R. (2003). Science 302, 650-654; Feng, Y., Degen, D., Wang, X., Gigliotti, M., Liu, S., Zhang, Y., Das, D., Michalchuk, T., Ebright, Y. W., Talaue, M., Connell, N., and Ebright, R. H. (2015). Structure 23, 1470-1481; Bae, B., Nayak, D., Ray, A., Mustaev, A., Landick, R., and Darst, S. A. (2015). Proc. Natl. Acad. Sci. USA 112, E4178-E4187). CBRs, for example, CBR703, can exhibit potent RNAP-inhibitory activity against RNAP from Gram-negative bacteria and potent antibacterial activity against Gram-negative bacterial species.

Na-aroyl-N-aryl-phenylalanianmides (AAPs) are another class of antibacterial agents known in the art that function by inhibiting RNAP through binding to the bridge-helix N-terminus target (Ebright, R. H., Ebright, Y., Mandal, S., Wilde, R., and Li, S. (2015) WO2015/120320; Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179). AAPs, for example, D-AAP-1 and IX-214, can exhibit potent RNAP-inhibitory activity against RNAP from Mycobacteria and potent antibacterial activity against Mycobacterial species.

Resistance to CBRs and AAPs arises from mutations that result in amino acid substitutions in, or immediately adjacent to the bridge-helix N-terminus target (Artsimovitch, I., Chu, C., Lynch, A. S., and Landick, R. (2003). Science 302, 650-654; Feng, Y., Degen, D., Wang, X., Gigliotti, M., Liu, S., Zhang, Y., Das, D., Michalchuk, T., Ebright, Y. W., Talaue, M., Connell, N., and Ebright, R. H. (2015). Structure 23, 1470-1481; Bae, B., Nayak, D., Ray, A., Mustaev, A., Landick, R., and Darst, S. A. (2015). Proc. Natl. Acad. Sci. USA 112, E4178-E4187; Ebright, R. H., Ebright, Y., Mandal, S., Wilde, R., and Li, S. (2015) WO2015/120320; Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179).

CBRs and AAPs exhibit no cross-resistance with rifamycins (Li, L., Chen, X., Fan, P., Mihalic, J. and Cutler, S. (2001) WO/2001/051456; Li, L., Chen, X., Cutler, S. and Mann, J. (2001) Pyrazole antimicrobial agents. WO/2001/082930; Artsimovitch, I., Chu, C., Lynch, A. S., and Landick, R. (2003). Science 302, 650-654; Feng, Y., Degen, D., Wang, X., Gigliotti, M., Liu, S., Zhang, Y., Das, D., Michalchuk, T., Ebright, Y. W., Talaue, M., Connell, N., and Ebright, R. H. (2015). Structure 23, 1470-1481; Bae, B., Nayak, D., Ray, A., Mustaev, A., Landick, R., and Darst, S. A. (2015). Proc. Natl. Acad. Sci. USA 112, E4178-E4187; Ebright, R. H., Ebright, Y., Mandal, S., Wilde, R., and Li, S. (2015) WO2015/120320; Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179).

γ includes any moiety that binds to the bridge-helix N-terminus of a bacterial RNA polymerase.

In certain embodiments, γ is selected from a CBR or an AAP.

In one embodiment, γ is a CBR.

In one embodiment, γ is a compound described in WO/2001/051456 or WO/2001/082930.

In one embodiment, γ is an AAP.

In one embodiment, γ is a compound described in WO2015/120320.

In one embodiment, γ is a compound according to general structural formula (I), or a salt thereof.

In one embodiment, γ is a compound according to general structural formula (II), or a salt thereof:

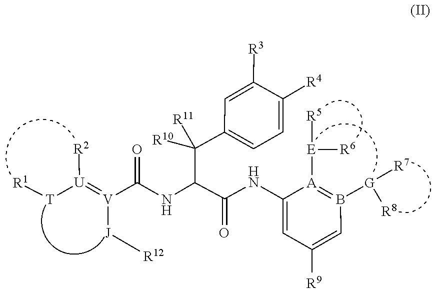

wherein:
T and U each is one of carbon and nitrogen;
E is carbon;
A and B each is one of carbon and nitrogen;
Y is one of carbon, nitrogen, oxygen, and sulfur;
Z is one of hydrogen, halogen, carbon, nitrogen, oxygen, and sulfur;
J is one of carbon and nitrogen, and J, together with T, U, and V forms part of a 6-membered cycle; or J is one of nitrogen, oxygen, sulfur, and selenium, and J, together with T, U, and V forms part of a 5-membered cycle;
R¹ and R² each independently is absent, hydrogen, hydroxy, or halogen, or is alkyl, alkoxy-substituted alkyl, amino-substituted alkyl, aryl-substituted alkyl, or alkoxy, each optionally substituted by halogen; or R¹ and R², together with T and U, form a cycle containing 4 to 9 atoms selected from carbon, nitrogen, oxygen, and sulfur;
R³ and R⁴ each independently is hydrogen, halogen, hydroxyl, amine, amide, ester, phosphate, or O-methylphosphate;
R⁵, R⁶, R⁷, and R⁸ each independently is absent, hydrogen, or halogen, or is alkyl, alkoxy-substituted alkyl, hydroxy-substituted alkyl, amino-substituted alkyl, aryl-substituted alkyl, or alkoxy, each optionally substituted by halogen; or R⁵ and R⁶, together with E, form a cycle containing 3 to 8 atoms selected from carbon, nitrogen, oxygen, and sulfur, said cycle optionally substituted with halogen, amino, alkyl, alkoxy-substituted alkyl, amino-substituted alkyl, aryl-substituted alkyl, alkoxy, acyl, or carbamidyl, each alkyl, alkoxy-substituted alkyl, amino-substituted alkyl, aryl-substituted alkyl, alkoxy, acyl, or carbamidyl optionally substituted by halogen; or R⁷ and R⁸, together with G, form a cycle containing 3 to 8 atoms selected from carbon, nitrogen, oxygen, and sulfur, said cycle optionally substituted with halogen, amino, alkyl, alkoxy-substituted alkyl, hydroxy-substituted alkyl, amino-substituted alkyl, or aryl-substituted alkyl, alkoxy, acyl, or carbamidyl, each alkyl, alkoxy-substituted alkyl, amino-substituted alkyl, aryl-substituted alkyl, alkoxy, acyl, or carbamidyl optionally substituted by halogen; or R⁶ and R⁷ are absent and E and G, together with A and B, form a cycle containing 4 to 9 atoms selected from carbon, nitrogen, oxygen, and sulfur, said cycle optionally substituted with halogen, amino, alkyl, alkoxy-substituted alkyl, amino-substituted alkyl, aryl-substituted alkyl, alkoxy, acyl, or carbamidyl, each alkyl, alkoxy-substituted alkyl, amino-substituted alkyl, aryl-substituted alkyl, alkoxy, acyl, or carbamidyl optionally substituted by halogen;
R⁹ is hydrogen or halogen;
R¹⁰ and R¹¹ each independently is one of hydrogen, halogen, alkyl, or alkoxy, said alkyl or alkoxy optionally substituted by halogen; or one of R¹⁰ and R¹¹ is deuterium, and the other is halogen, alkyl, or alkoxy, said alkyl or alkoxy optionally substituted by halogen; or each of R¹⁰ and R¹¹ is deuterium; and
R¹² is absent, hydrogen, or halogen;
   or a tautomer or salt thereof.

In one embodiment, γ is a compound according to general structural formula (II),
wherein:
T and U each is one of carbon and nitrogen;
E is carbon;
A and B each is one of carbon and nitrogen;
Y is one of carbon, nitrogen, oxygen, and sulfur;
Z is one of hydrogen, halogen, carbon, nitrogen, oxygen, and sulfur;
J is one of carbon and nitrogen, and J, together with T, U, and V forms part of a 6-membered cycle; or J is one of nitrogen, oxygen, sulfur, and selenium, and J, together with T, U, and V forms part of a 5-membered cycle;
R¹ and R² each independently is absent, hydrogen, hydroxy, or halogen, or is (C₁-C₆) alkyl, (C₁-C₆) alkoxy-substituted (C₁-C₆)alkyl, amino-substituted (C₁-C₆)alkyl, aryl-substituted (C₁-C₆) alkyl, or (C₁-C₆) alkoxy, each optionally substituted by halogen; or R¹ and R², together with T and U, form a cycle containing 4 to 9 atoms selected from carbon, nitrogen, oxygen, and sulfur;
R³ and R⁴ each independently is hydrogen, halogen, hydroxyl, amine, amide, ester, phosphate, or O-methylphosphate;
R⁵, R⁶, R⁷, and R⁸ each independently is absent, hydrogen, or halogen, or is (C₁-C₆) alkyl, (C₁-C₆) alkoxy-substituted (C₁-C₆) alkyl, hydroxy-substituted (C₁-C₆)alkyl, amino-substituted (C₁-C₆)alkyl, aryl-substituted (C₁-C₆) alkyl, or (C₁-C₆) alkoxy, each optionally substituted by halogen; or R⁵ and R⁶, together with E, form a cycle containing 3 to 8 atoms selected from carbon, nitrogen, oxygen, and sulfur, said cycle optionally substituted with halogen, amino, (C1-C₆) alkyl, (C₁-C₆) alkoxy-substituted (C₁-C₆)alkyl, amino-substituted (C₁-C₆)alkyl, aryl-substituted (C₁-C₆) alkyl, (C₁-C₆) alkoxy, acyl, or carbamidyl, each (C₁-C₆) alkyl, (C₁-C₆) alkoxy-substituted (C₁-C₆)alkyl, amino-substituted (C₁-C₆)alkyl, aryl-substituted (C₁-C₆) alkyl, (C₁-C₆) alkoxy, acyl, or carbamidyl optionally substituted by halogen; or R⁷ and R⁸, together with G, form a cycle containing 3 to 8 atoms selected from carbon, nitrogen, oxygen, and sulfur, said cycle optionally substituted with halogen, amino, alkyl, (C₁-C₆) alkoxy-substituted (C₁-C₆) alkyl, hydroxy-substituted (C₁-C₆)alkyl, amino-substituted (C₁-C₆) alkyl, or aryl-substituted (C₁-C₆) alkyl, alkoxy, acyl, or carbamidyl, each (C₁-C₆) alkyl, (C₁-C₆) alkoxy-substituted (C₁-C₆) alkyl, amino-substituted (C₁-C₆)alkyl, aryl-substituted (C₁-C₆) alkyl, (C₁-C₆) alkoxy, acyl, or carbamidyl optionally substituted by halogen; or R⁶ and R⁷ are absent and E and G, together with A and B, form a cycle containing 4 to 9 atoms selected from carbon, nitrogen, oxygen, and sulfur, said cycle optionally substituted with halogen, amino, (C₁-C₆) alkyl, (C₁-C₆) alkoxy-substituted (C₁-C₆)alkyl, amino-substituted (C₁-C₆)alkyl, aryl-substituted (C₁-C₆) alkyl, (C₁-C₆) alkoxy, acyl, or carbamidyl, each (C₁-C₆) alkyl, (C₁-C₆) alkoxy-substituted (C₁-C₆) alkyl, amino-substituted (C₁-C₆)alkyl, aryl-substituted (C₁-C₆) alkyl, (C₁-C₆) alkoxy, acyl, or carbamidyl optionally substituted by halogen;
R⁹ is hydrogen or halogen;
R¹⁰ and R¹¹ each independently is one of hydrogen, halogen, (C₁-C₆) alkyl, or (C₁-C₆) alkoxy, said (C₁-C₆) alkyl or (C₁-C₆) alkoxy optionally substituted by halogen; or one of R¹⁰ and R¹¹ is deuterium, and the other is halogen, (C₁-C₆) alkyl, or (C₁-C₆) alkoxy, said (C₁-C₆) alkyl or (C₁-C₆) alkoxy optionally substituted by halogen; or each of R¹⁰ and R¹¹ is deuterium; and
R¹² is absent, hydrogen, or halogen;
   or a tautomer or salt thereof.

It is understood that γ can be connected to β through any synthetically feasable position on γ. Alternatively, γ can be connected to β by removing one or more atoms from γ to provide a residue of γ having an open valence suitable for bonding with β. Synthetic reagents and techniques for attaching a γ to β are known and available. In one embodiment, a compound of formula (II) or (IIa) can be attached to the remainder of a compound of formula (I) through variable A, B, E, R⁵, R⁶, R⁷, R⁸, R⁵-R⁶ or R⁷-R⁸.

In one embodiment, γ is a compound of formula (IIa) or a tautomer or salt thereof:

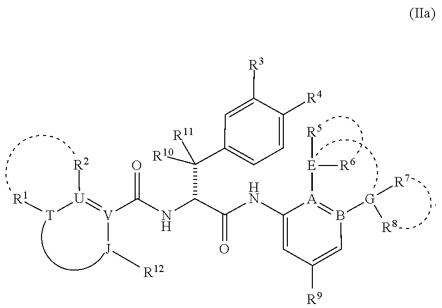

In one embodiment, γ is a compound selected from the group consisting of:

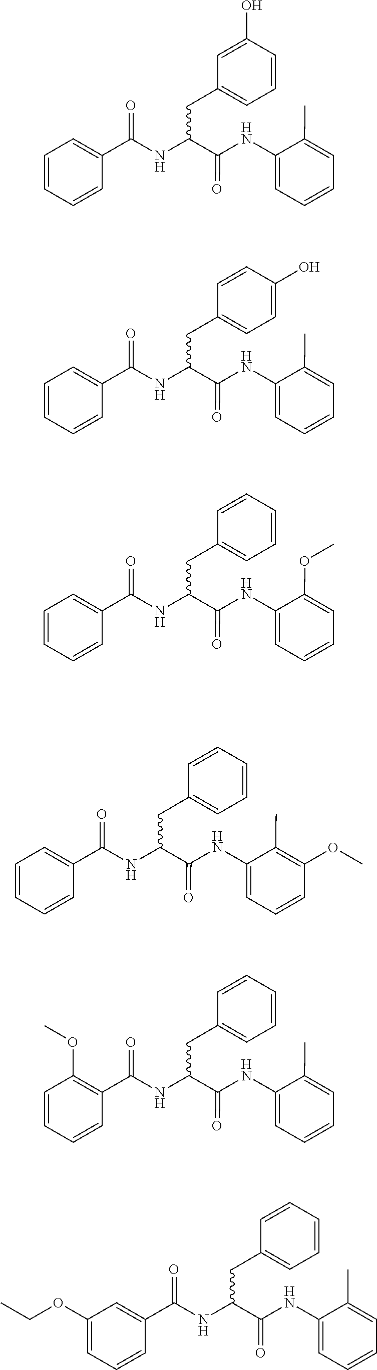

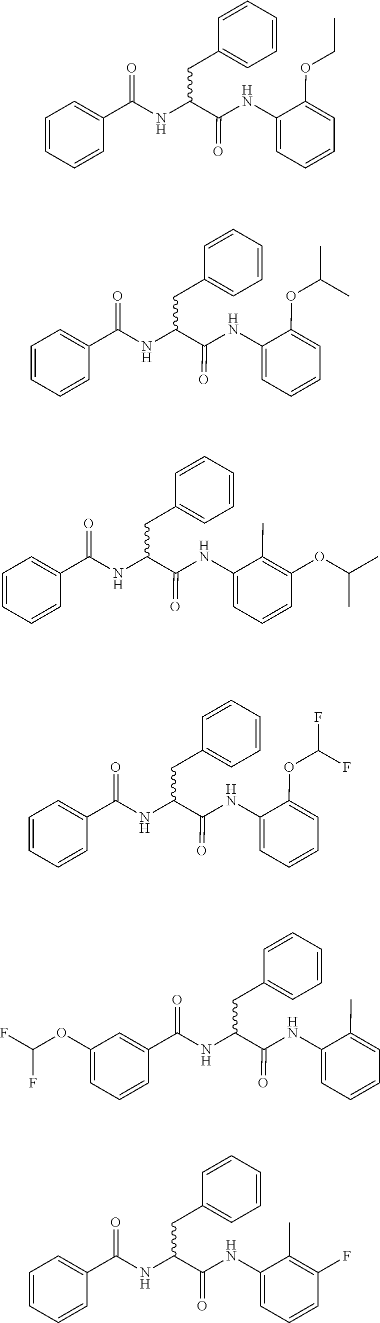 [SMILES: CCOc1ccccc1NC(=O)C(Cc1ccccc1)NC(=O)c1ccccc1 ; CC(C)Oc1ccccc1NC(=O)C(Cc1ccccc1)NC(=O)c1ccccc1 ; Cc1c(NC(=O)C(Cc2ccccc2)NC(=O)c2ccccc2)cccc1OC(C)C ; O=C(NC(Cc1ccccc1)C(=O)Nc1ccccc1OC(F)F)c1ccccc1 ; Cc1ccccc1NC(=O)C(Cc1ccccc1)NC(=O)c1cccc(OC(F)F)c1 ; Cc1c(F)cccc1NC(=O)C(Cc1ccccc1)NC(=O)c1ccccc1]

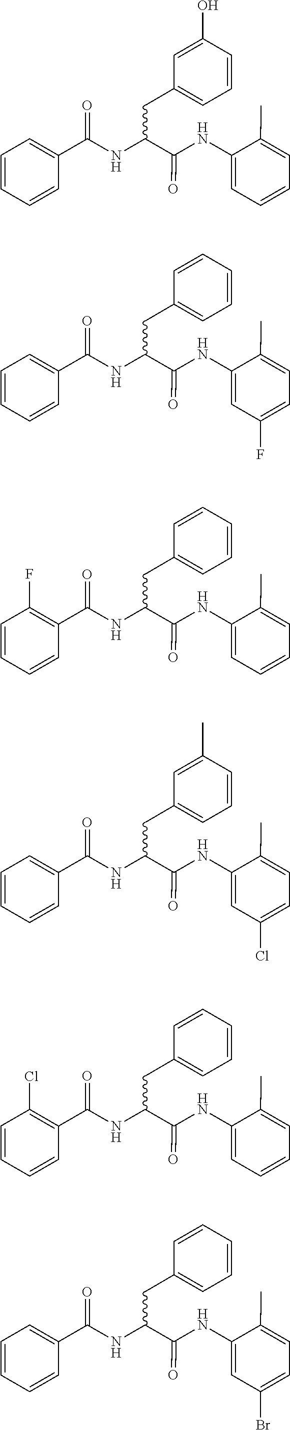 [SMILES: Cc1ccccc1NC(=O)C(Cc1cccc(O)c1)NC(=O)c1ccccc1 ; Cc1ccc(F)cc1NC(=O)C(Cc1ccccc1)NC(=O)c1ccccc1 ; Cc1ccccc1NC(=O)C(Cc1ccccc1)NC(=O)c1ccccc1F ; Cc1cccc(CC(NC(=O)c2ccccc2)C(=O)Nc2cc(Cl)ccc2C)c1 ; Cc1ccccc1NC(=O)C(Cc1ccccc1)NC(=O)c1ccccc1Cl ; Cc1ccc(Br)cc1NC(=O)C(Cc1ccccc1)NC(=O)c1ccccc1]

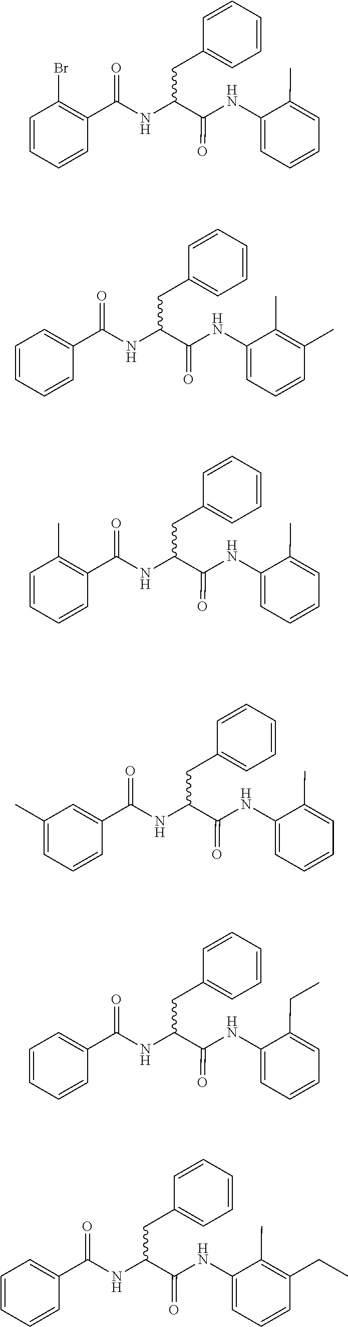

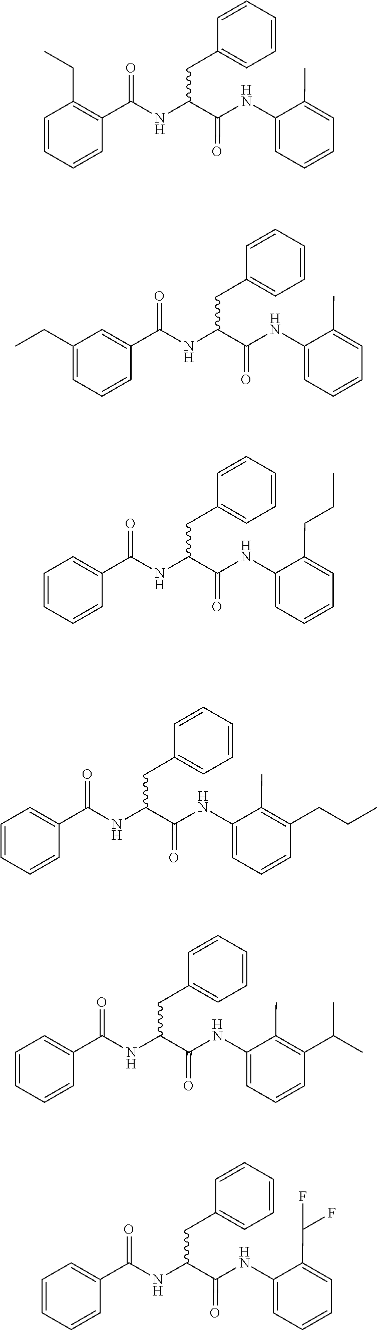 [SMILES: CCc1ccccc1C(=O)NC(Cc1ccccc1)C(=O)Nc1ccccc1C ; CCc1cccc(C(=O)NC(Cc2ccccc2)C(=O)Nc2ccccc2C)c1 ; CCCc1ccccc1NC(=O)C(Cc1ccccc1)NC(=O)c1ccccc1 ; CCCc1cccc(NC(=O)C(Cc2ccccc2)NC(=O)c2ccccc2)c1C ; Cc1c(NC(=O)C(Cc2ccccc2)NC(=O)c2ccccc2)cccc1C(C)C ; O=C(NC(Cc1ccccc1)C(=O)Nc1ccccc1C(F)F)c1ccccc1]

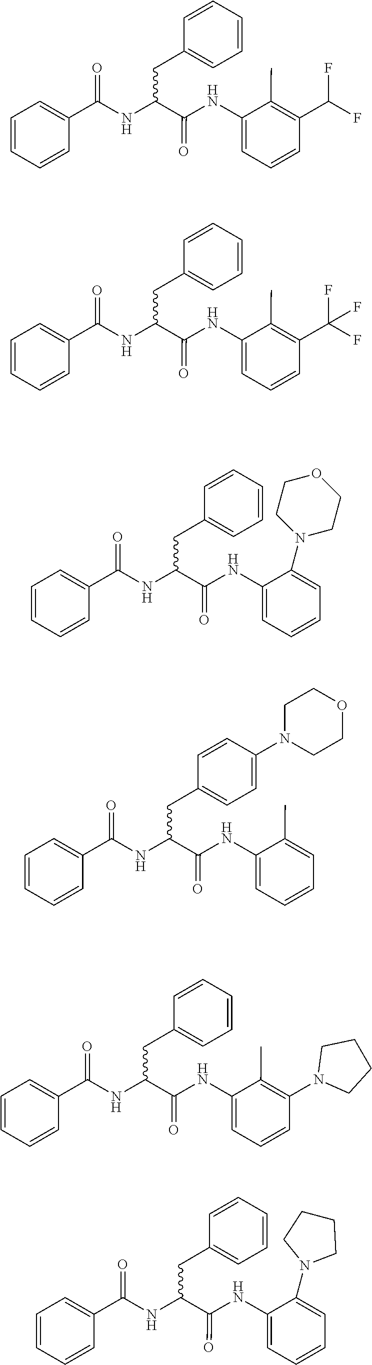 [SMILES: Cc1c(NC(=O)C(Cc2ccccc2)NC(=O)c2ccccc2)cccc1C(F)F ; Cc1c(NC(=O)C(Cc2ccccc2)NC(=O)c2ccccc2)cccc1C(F)(F)F ; O=C(NC(Cc1ccccc1)C(=O)Nc1ccccc1N1CCOCC1)c1ccccc1 ; Cc1ccccc1NC(=O)C(Cc1ccc(N2CCOCC2)cc1)NC(=O)c1ccccc1 ; Cc1c(NC(=O)C(Cc2ccccc2)NC(=O)c2ccccc2)cccc1N1CCCC1 ; O=C(NC(Cc1ccccc1)C(=O)Nc1ccccc1N1CCCC1)c1ccccc1]

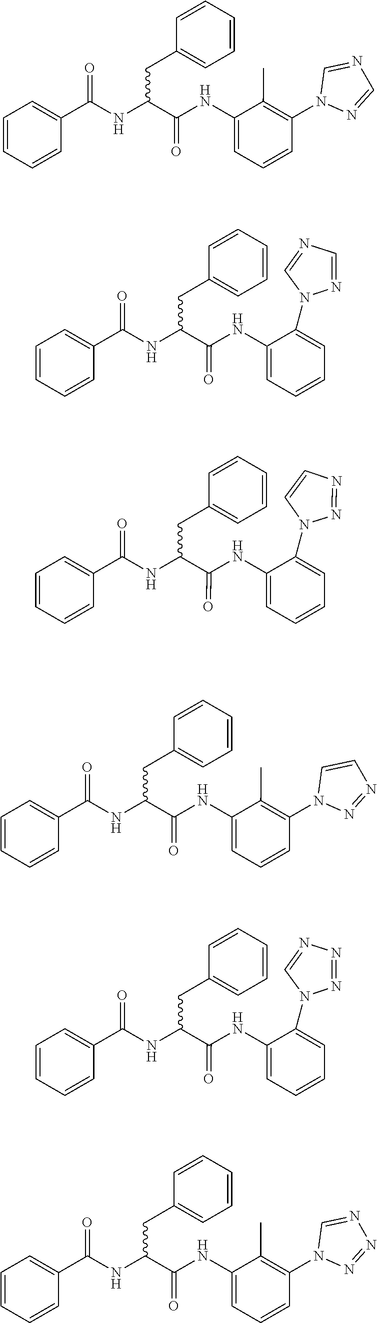 [SMILES: Cc1c(NC(=O)C(Cc2ccccc2)NC(=O)c2ccccc2)cccc1-n1cncn1 ; O=C(NC(Cc1ccccc1)C(=O)Nc1ccccc1-n1cncn1)c1ccccc1 ; O=C(NC(Cc1ccccc1)C(=O)Nc1ccccc1-n1ccnn1)c1ccccc1 ; Cc1c(NC(=O)C(Cc2ccccc2)NC(=O)c2ccccc2)cccc1-n1ccnn1 ; O=C(NC(Cc1ccccc1)C(=O)Nc1ccccc1-n1cnnn1)c1ccccc1 ; Cc1c(NC(=O)C(Cc2ccccc2)NC(=O)c2ccccc2)cccc1-n1cnnn1]

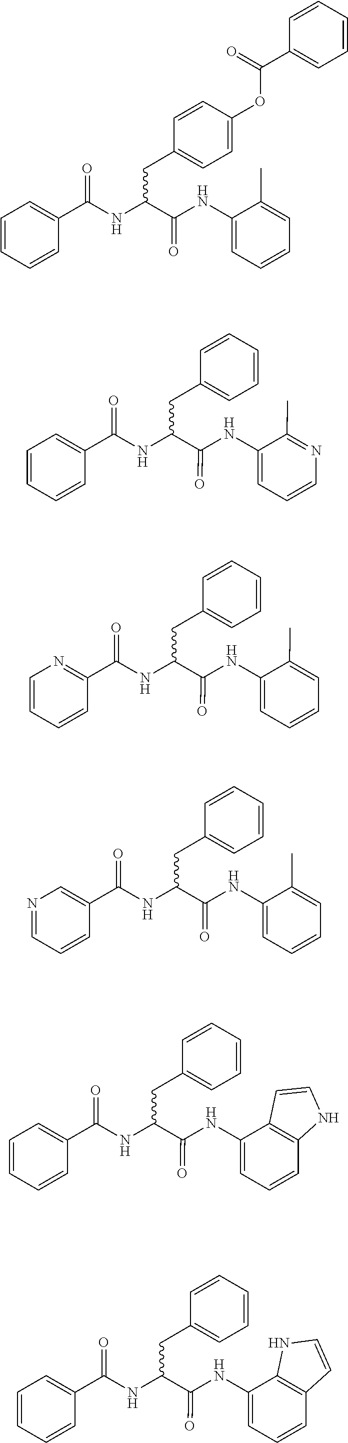 [SMILES: Cc1ccccc1NC(=O)C(Cc1ccc(OC(=O)c2ccccc2)cc1)NC(=O)c1ccccc1 ; Cc1ncccc1NC(=O)C(Cc1ccccc1)NC(=O)c1ccccc1 ; Cc1ccccc1NC(=O)C(Cc1ccccc1)NC(=O)c1ccccn1 ; Cc1ccccc1NC(=O)C(Cc1ccccc1)NC(=O)c1cccnc1 ; O=C(NC(Cc1ccccc1)C(=O)Nc1cccc2[nH]ccc12)c1ccccc1 ; O=C(NC(Cc1ccccc1)C(=O)Nc1cccc2cc[nH]c12)c1ccccc1]

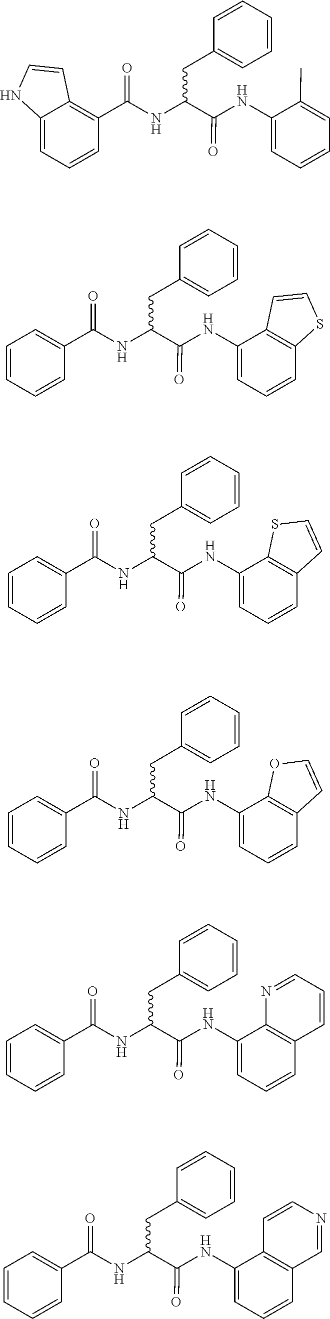 [SMILES: Cc1ccccc1NC(=O)C(Cc1ccccc1)NC(=O)c1cccc2[nH]ccc12 ; O=C(NC(Cc1ccccc1)C(=O)Nc1cccc2sccc12)c1ccccc1 ; O=C(NC(Cc1ccccc1)C(=O)Nc1cccc2ccsc12)c1ccccc1 ; O=C(NC(Cc1ccccc1)C(=O)Nc1cccc2ccoc12)c1ccccc1 ; O=C(NC(Cc1ccccc1)C(=O)Nc1cccc2cccnc12)c1ccccc1 ; O=C(NC(Cc1ccccc1)C(=O)Nc1cccc2cnccc12)c1ccccc1]

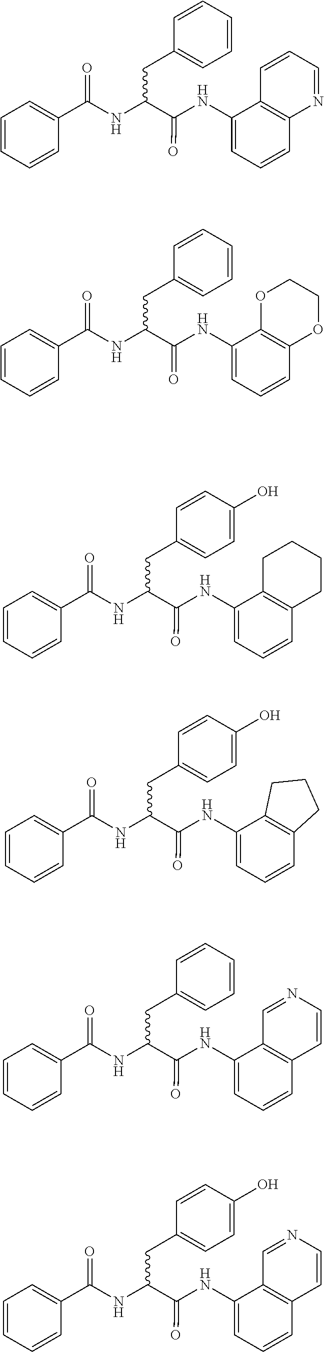 [SMILES: O=C(NC(Cc1ccccc1)C(=O)Nc1cccc2ncccc12)c1ccccc1 ; O=C(NC(Cc1ccccc1)C(=O)Nc1cccc2c1OCCO2)c1ccccc1 ; O=C(NC(Cc1ccc(O)cc1)C(=O)Nc1cccc2c1CCCC2)c1ccccc1 ; O=C(NC(Cc1ccc(O)cc1)C(=O)Nc1cccc2c1CCC2)c1ccccc1 ; O=C(NC(Cc1ccccc1)C(=O)Nc1cccc2ccncc12)c1ccccc1 ; O=C(NC(Cc1ccc(O)cc1)C(=O)Nc1cccc2ccncc12)c1ccccc1]

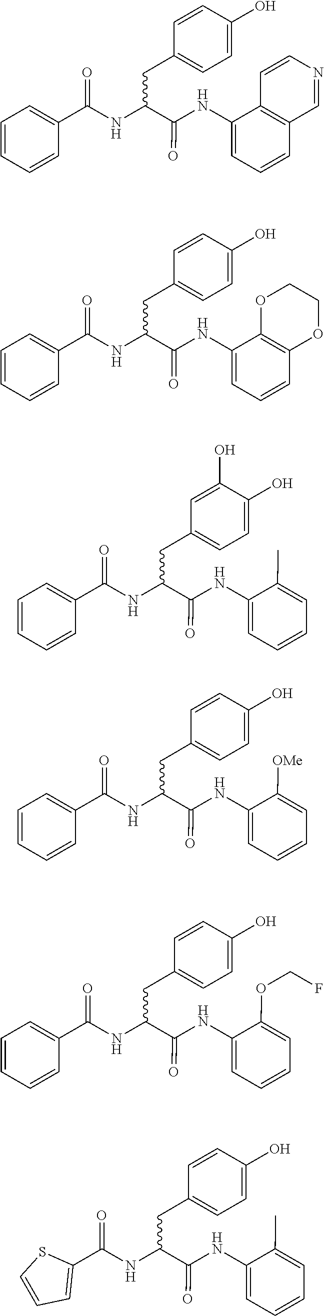

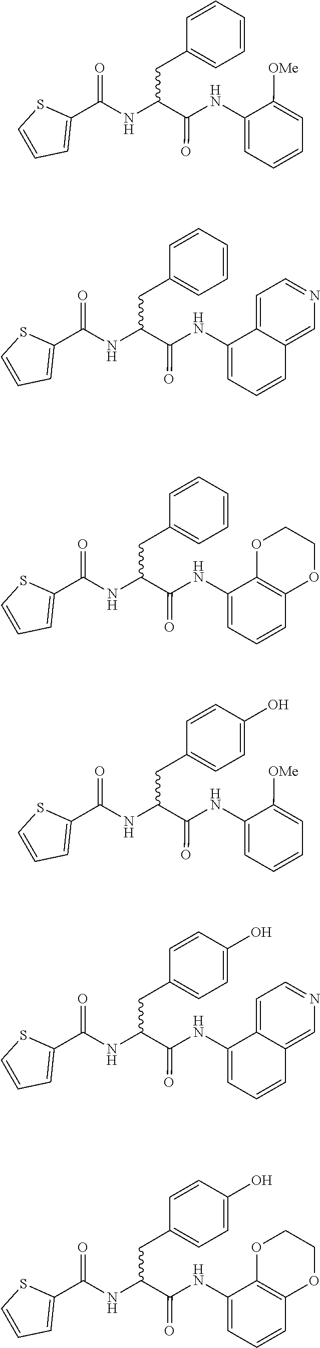 [SMILES: COc1ccccc1NC(=O)C(Cc1ccccc1)NC(=O)c1cccs1 ; O=C(NC(Cc1ccccc1)C(=O)Nc1cccc2cnccc12)c1cccs1 ; O=C(NC(Cc1ccccc1)C(=O)Nc1cccc2c1OCCO2)c1cccs1 ; COc1ccccc1NC(=O)C(Cc1ccc(O)cc1)NC(=O)c1cccs1 ; O=C(NC(Cc1ccc(O)cc1)C(=O)Nc1cccc2cnccc12)c1cccs1 ; O=C(NC(Cc1ccc(O)cc1)C(=O)Nc1cccc2c1OCCO2)c1cccs1]

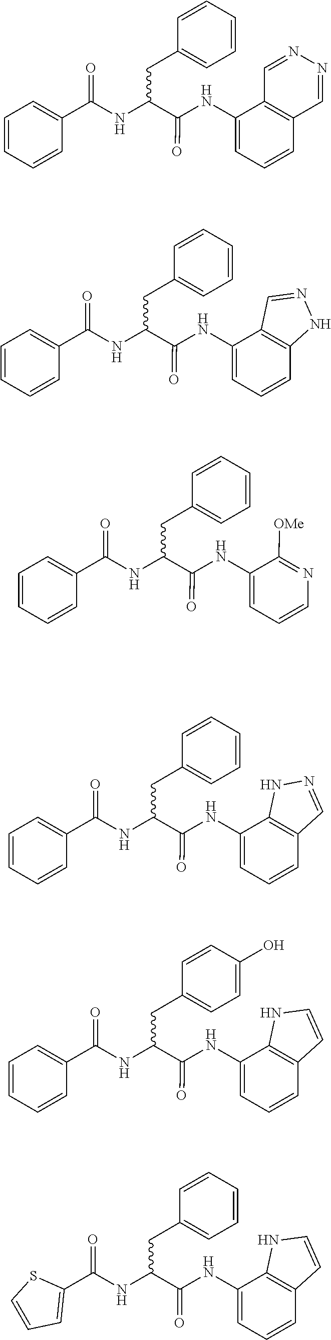 [SMILES: O=C(NC(Cc1ccccc1)C(=O)Nc1cccc2cnncc12)c1ccccc1 ; O=C(NC(Cc1ccccc1)C(=O)Nc1cccc2[nH]ncc12)c1ccccc1 ; COc1ncccc1NC(=O)C(Cc1ccccc1)NC(=O)c1ccccc1 ; O=C(NC(Cc1ccccc1)C(=O)Nc1cccc2cn[nH]c12)c1ccccc1 ; O=C(NC(Cc1ccc(O)cc1)C(=O)Nc1cccc2cc[nH]c12)c1ccccc1 ; O=C(NC(Cc1ccccc1)C(=O)Nc1cccc2cc[nH]c12)c1cccs1]

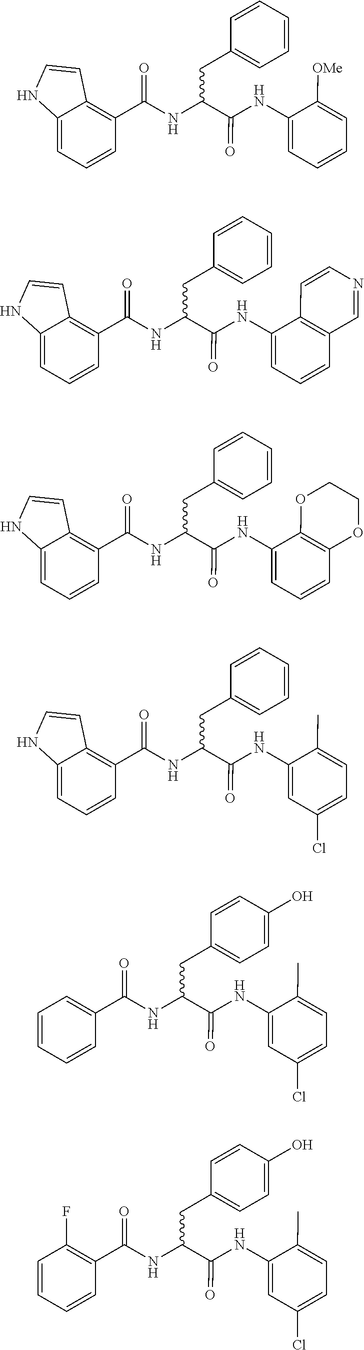 [SMILES: COc1ccccc1NC(=O)C(Cc1ccccc1)NC(=O)c1cccc2[nH]ccc12 ; O=C(NC(Cc1ccccc1)C(=O)Nc1cccc2cnccc12)c1cccc2[nH]ccc12 ; O=C(NC(Cc1ccccc1)C(=O)Nc1cccc2c1OCCO2)c1cccc2[nH]ccc12 ; Cc1ccc(Cl)cc1NC(=O)C(Cc1ccccc1)NC(=O)c1cccc2[nH]ccc12 ; Cc1ccc(Cl)cc1NC(=O)C(Cc1ccc(O)cc1)NC(=O)c1ccccc1 ; Cc1ccc(Cl)cc1NC(=O)C(Cc1ccc(O)cc1)NC(=O)c1ccccc1F]

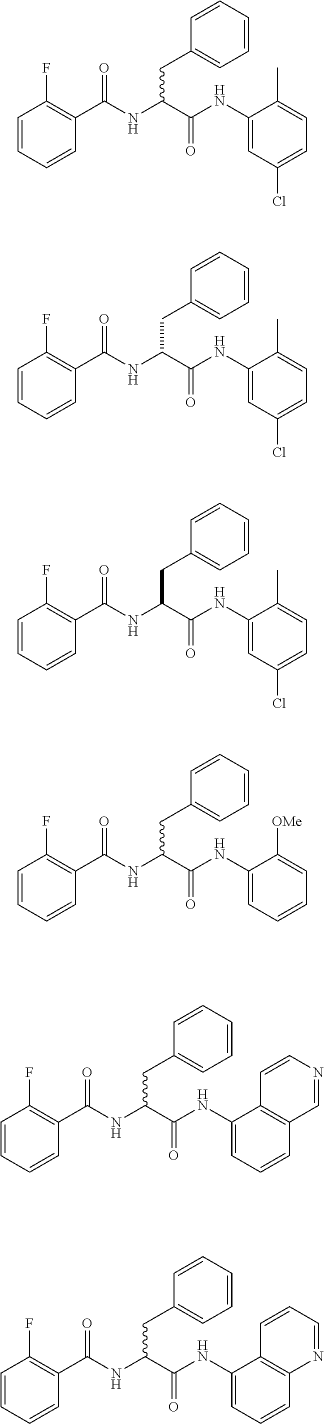 [SMILES: Cc1ccc(Cl)cc1NC(=O)C(Cc1ccccc1)NC(=O)c1ccccc1F ; Cc1ccc(Cl)cc1NC(=O)[C@@H](Cc1ccccc1)NC(=O)c1ccccc1F ; Cc1ccc(Cl)cc1NC(=O)[C@H](Cc1ccccc1)NC(=O)c1ccccc1F ; COc1ccccc1NC(=O)C(Cc1ccccc1)NC(=O)c1ccccc1F ; O=C(NC(Cc1ccccc1)C(=O)Nc1cccc2cnccc12)c1ccccc1F ; O=C(NC(Cc1ccccc1)C(=O)Nc1cccc2ncccc12)c1ccccc1F]

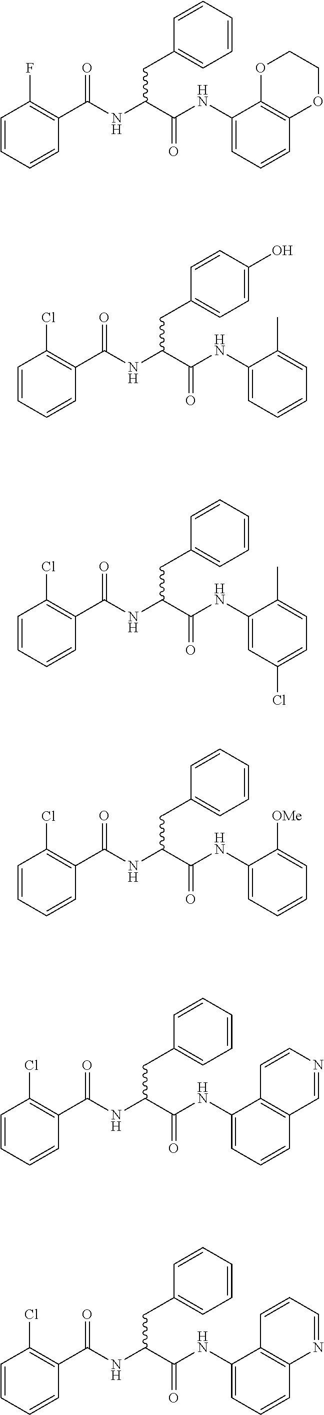 [SMILES: O=C(NC(Cc1ccccc1)C(=O)Nc1cccc2c1OCCO2)c1ccccc1F ; Cc1ccccc1NC(=O)C(Cc1ccc(O)cc1)NC(=O)c1ccccc1Cl ; Cc1ccc(Cl)cc1NC(=O)C(Cc1ccccc1)NC(=O)c1ccccc1Cl ; COc1ccccc1NC(=O)C(Cc1ccccc1)NC(=O)c1ccccc1Cl ; O=C(NC(Cc1ccccc1)C(=O)Nc1cccc2cnccc12)c1ccccc1Cl ; O=C(NC(Cc1ccccc1)C(=O)Nc1cccc2ncccc12)c1ccccc1Cl]

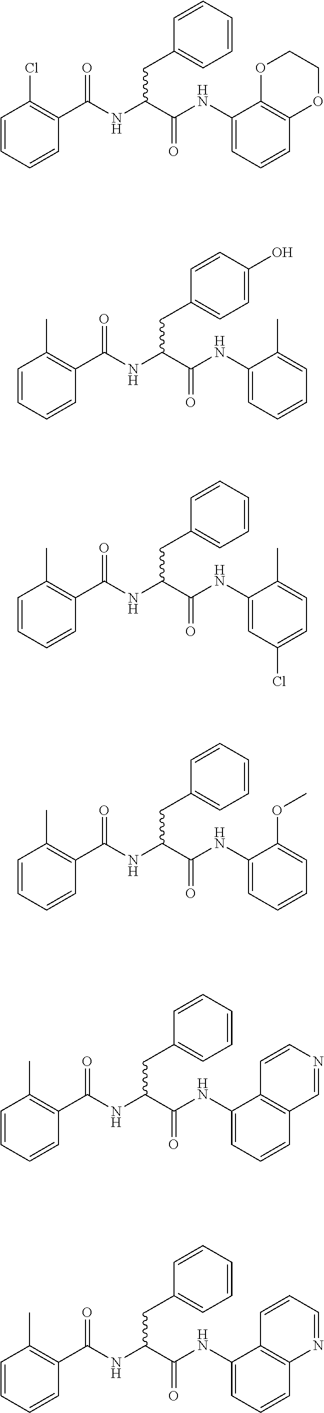 [SMILES: O=C(NC(Cc1ccccc1)C(=O)Nc1cccc2c1OCCO2)c1ccccc1Cl ; Cc1ccccc1NC(=O)C(Cc1ccc(O)cc1)NC(=O)c1ccccc1C ; Cc1ccc(Cl)cc1NC(=O)C(Cc1ccccc1)NC(=O)c1ccccc1C ; COc1ccccc1NC(=O)C(Cc1ccccc1)NC(=O)c1ccccc1C ; Cc1ccccc1C(=O)NC(Cc1ccccc1)C(=O)Nc1cccc2cnccc12 ; Cc1ccccc1C(=O)NC(Cc1ccccc1)C(=O)Nc1cccc2ncccc12]

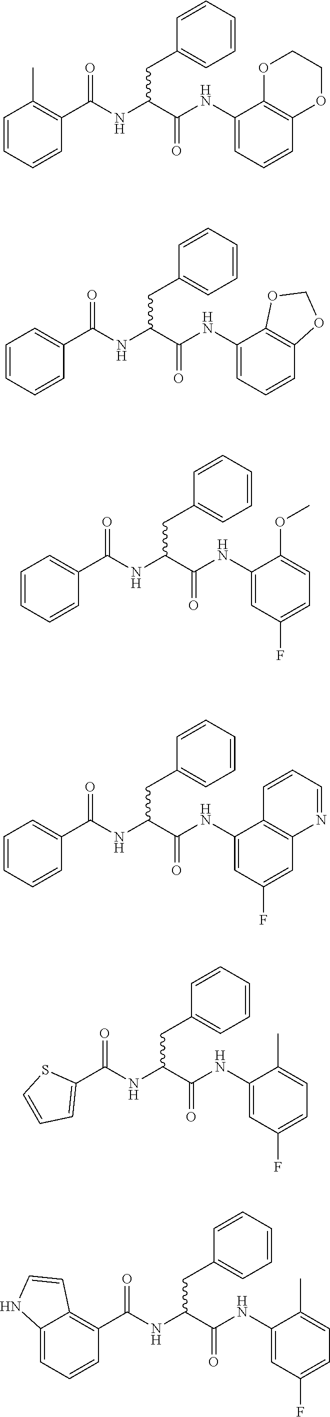 [SMILES: Cc1ccccc1C(=O)NC(Cc1ccccc1)C(=O)Nc1cccc2c1OCCO2 ; O=C(NC(Cc1ccccc1)C(=O)Nc1cccc2c1OCO2)c1ccccc1 ; COc1ccc(F)cc1NC(=O)C(Cc1ccccc1)NC(=O)c1ccccc1 ; O=C(NC(Cc1ccccc1)C(=O)Nc1cc(F)cc2ncccc12)c1ccccc1 ; Cc1ccc(F)cc1NC(=O)C(Cc1ccccc1)NC(=O)c1cccs1 ; Cc1ccc(F)cc1NC(=O)C(Cc1ccccc1)NC(=O)c1cccc2[nH]ccc12]

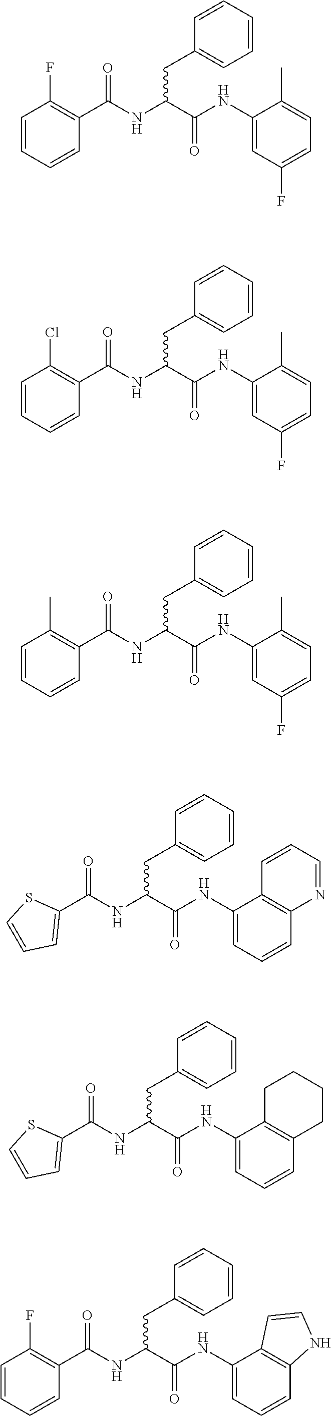 [SMILES: Cc1ccc(F)cc1NC(=O)C(Cc1ccccc1)NC(=O)c1ccccc1F ; Cc1ccc(F)cc1NC(=O)C(Cc1ccccc1)NC(=O)c1ccccc1Cl ; Cc1ccc(F)cc1NC(=O)C(Cc1ccccc1)NC(=O)c1ccccc1C ; O=C(NC(Cc1ccccc1)C(=O)Nc1cccc2ncccc12)c1cccs1 ; O=C(NC(Cc1ccccc1)C(=O)Nc1cccc2c1CCCC2)c1cccs1 ; O=C(NC(Cc1ccccc1)C(=O)Nc1cccc2[nH]ccc12)c1ccccc1F]

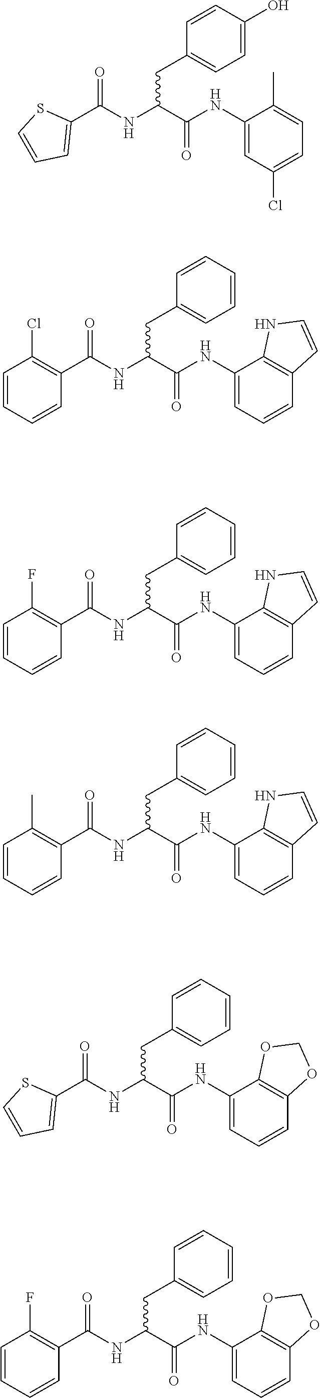 [SMILES: Cc1ccc(Cl)cc1NC(=O)C(Cc1ccc(O)cc1)NC(=O)c1cccs1 ; O=C(NC(Cc1ccccc1)C(=O)Nc1cccc2cc[nH]c12)c1ccccc1Cl ; O=C(NC(Cc1ccccc1)C(=O)Nc1cccc2cc[nH]c12)c1ccccc1F ; Cc1ccccc1C(=O)NC(Cc1ccccc1)C(=O)Nc1cccc2cc[nH]c12 ; O=C(NC(Cc1ccccc1)C(=O)Nc1cccc2c1OCO2)c1cccs1 ; O=C(NC(Cc1ccccc1)C(=O)Nc1cccc2c1OCO2)c1ccccc1F]

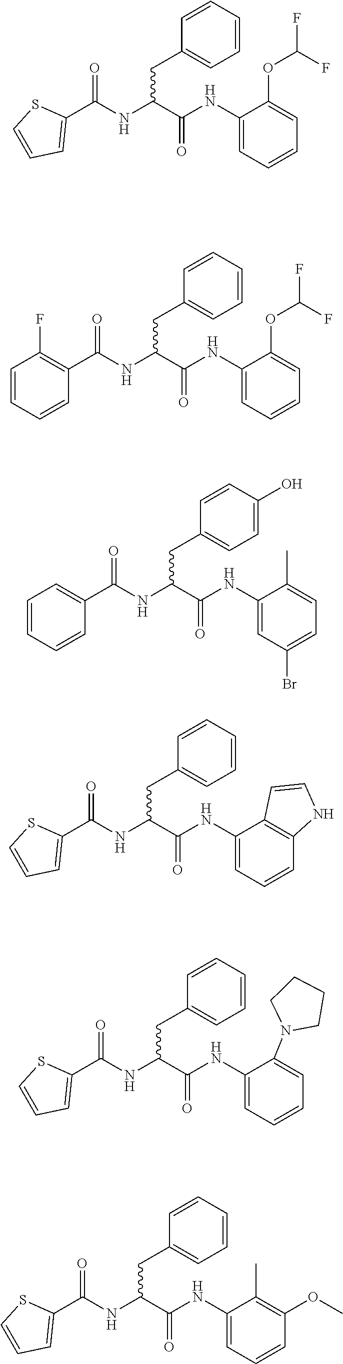 [SMILES: O=C(NC(Cc1ccccc1)C(=O)Nc1ccccc1OC(F)F)c1cccs1 ; O=C(NC(Cc1ccccc1)C(=O)Nc1ccccc1OC(F)F)c1ccccc1F ; Cc1ccc(Br)cc1NC(=O)C(Cc1ccc(O)cc1)NC(=O)c1ccccc1 ; O=C(NC(Cc1ccccc1)C(=O)Nc1cccc2[nH]ccc12)c1cccs1 ; O=C(NC(Cc1ccccc1)C(=O)Nc1ccccc1N1CCCC1)c1cccs1 ; COc1cccc(NC(=O)C(Cc2ccccc2)NC(=O)c2cccs2)c1C]

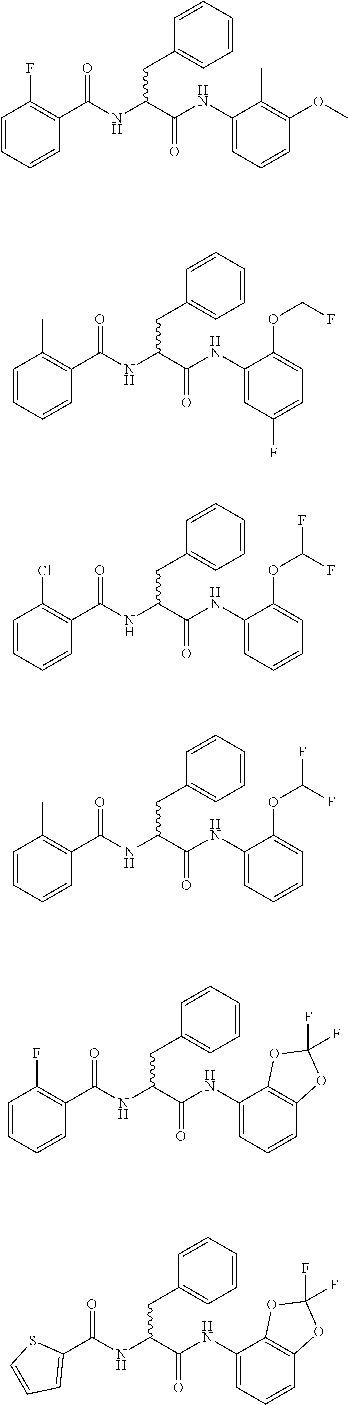 [SMILES: COc1cccc(NC(=O)C(Cc2ccccc2)NC(=O)c2ccccc2F)c1C ; Cc1ccccc1C(=O)NC(Cc1ccccc1)C(=O)Nc1cc(F)ccc1OCF ; O=C(NC(Cc1ccccc1)C(=O)Nc1ccccc1OC(F)F)c1ccccc1Cl ; Cc1ccccc1C(=O)NC(Cc1ccccc1)C(=O)Nc1ccccc1OC(F)F ; O=C(NC(Cc1ccccc1)C(=O)Nc1cccc2c1OC(F)(F)O2)c1ccccc1F ; O=C(NC(Cc1ccccc1)C(=O)Nc1cccc2c1OC(F)(F)O2)c1cccs1]

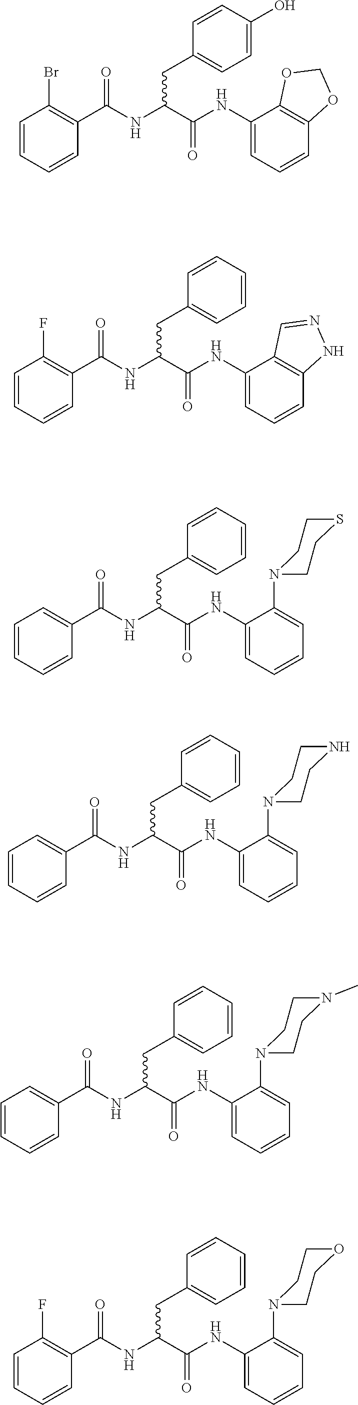 [SMILES: O=C(NC(Cc1ccc(O)cc1)C(=O)Nc1cccc2c1OCO2)c1ccccc1Br ; O=C(NC(Cc1ccccc1)C(=O)Nc1cccc2[nH]ncc12)c1ccccc1F ; O=C(NC(Cc1ccccc1)C(=O)Nc1ccccc1N1CCSCC1)c1ccccc1 ; O=C(NC(Cc1ccccc1)C(=O)Nc1ccccc1N1CCNCC1)c1ccccc1 ; CN1CCN(c2ccccc2NC(=O)C(Cc2ccccc2)NC(=O)c2ccccc2)CC1 ; O=C(NC(Cc1ccccc1)C(=O)Nc1ccccc1N1CCOCC1)c1ccccc1F]

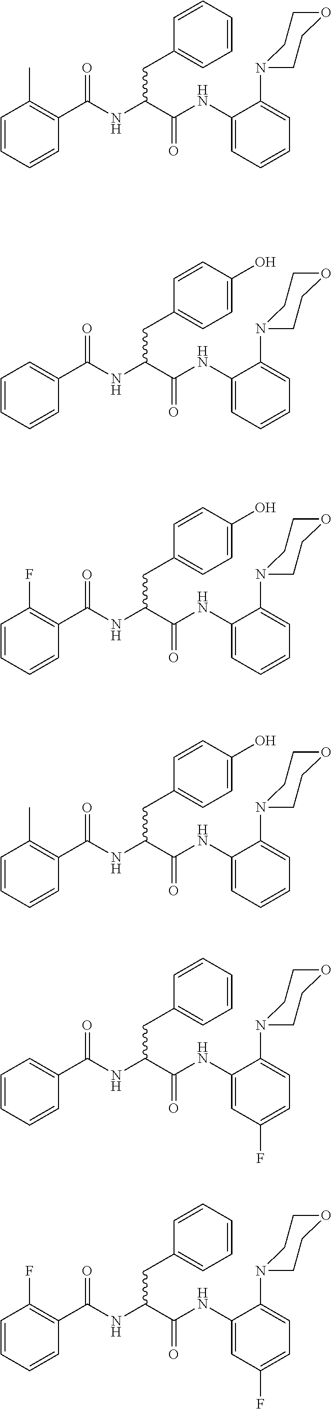 [SMILES: Cc1ccccc1C(=O)NC(Cc1ccccc1)C(=O)Nc1ccccc1N1CCOCC1 ; O=C(NC(Cc1ccc(O)cc1)C(=O)Nc1ccccc1N1CCOCC1)c1ccccc1 ; O=C(NC(Cc1ccc(O)cc1)C(=O)Nc1ccccc1N1CCOCC1)c1ccccc1F ; Cc1ccccc1C(=O)NC(Cc1ccc(O)cc1)C(=O)Nc1ccccc1N1CCOCC1 ; O=C(NC(Cc1ccccc1)C(=O)Nc1cc(F)ccc1N1CCOCC1)c1ccccc1 ; O=C(NC(Cc1ccccc1)C(=O)Nc1cc(F)ccc1N1CCOCC1)c1ccccc1F]

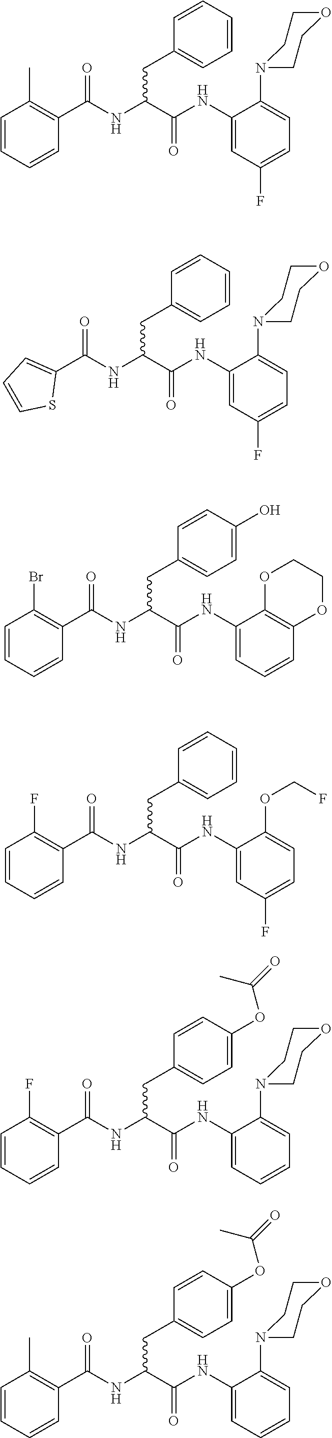

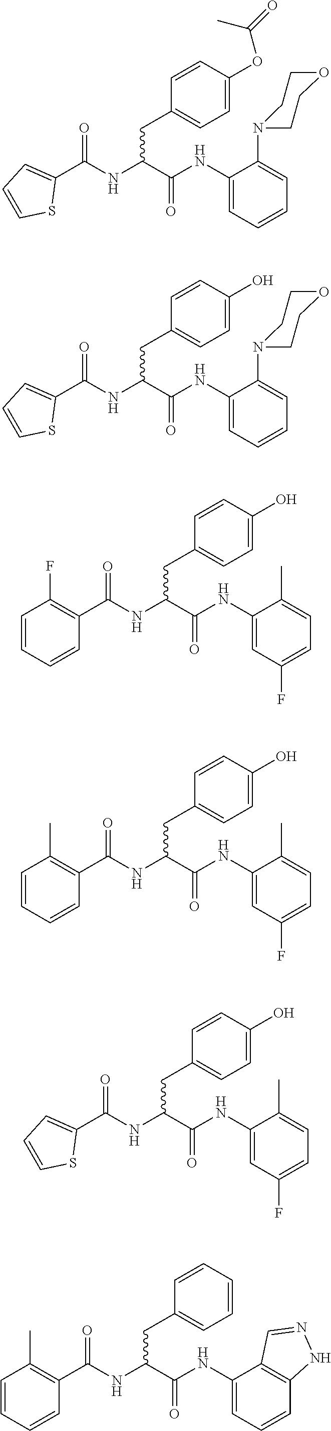 [SMILES: CC(=O)Oc1ccc(CC(NC(=O)c2cccs2)C(=O)Nc2ccccc2N2CCOCC2)cc1 ; O=C(NC(Cc1ccc(O)cc1)C(=O)Nc1ccccc1N1CCOCC1)c1cccs1 ; Cc1ccc(F)cc1NC(=O)C(Cc1ccc(O)cc1)NC(=O)c1ccccc1F ; Cc1ccc(F)cc1NC(=O)C(Cc1ccc(O)cc1)NC(=O)c1ccccc1C ; Cc1ccc(F)cc1NC(=O)C(Cc1ccc(O)cc1)NC(=O)c1cccs1 ; Cc1ccccc1C(=O)NC(Cc1ccccc1)C(=O)Nc1cccc2[nH]ncc12]

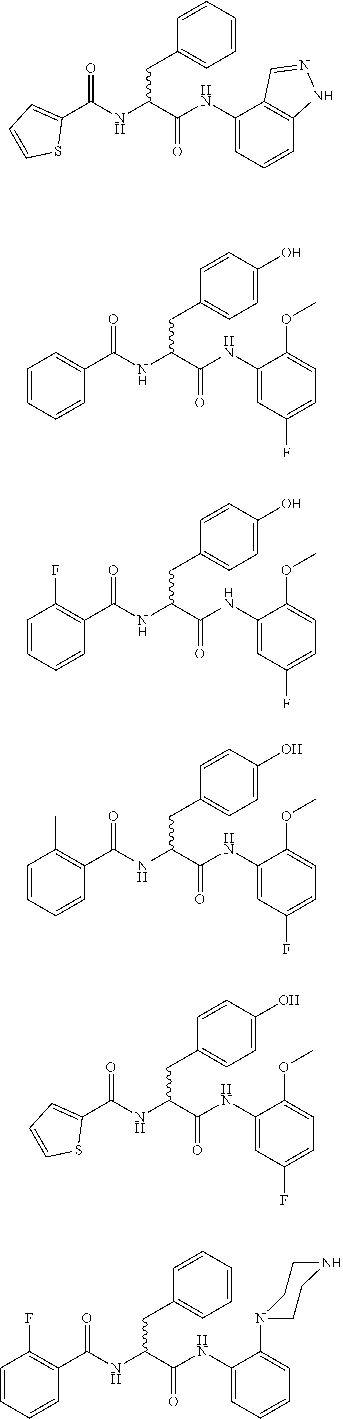

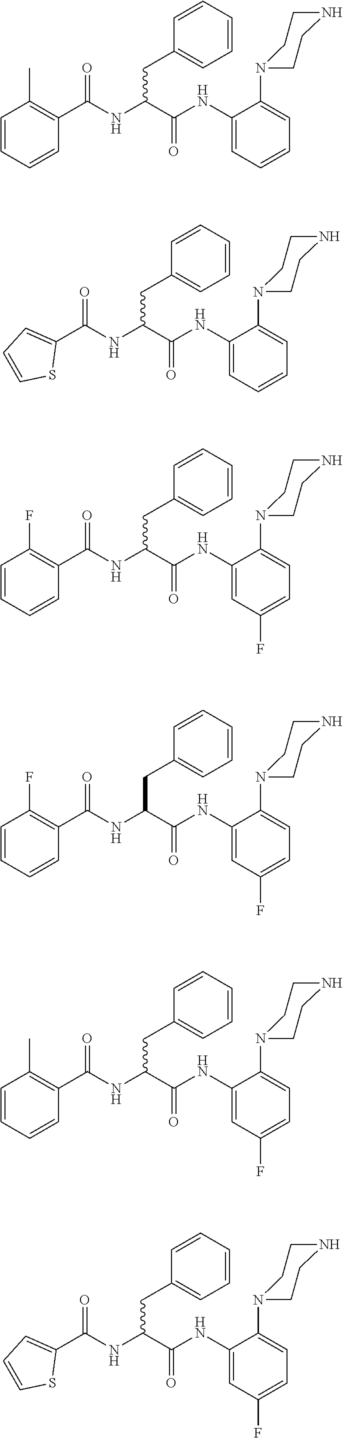 [SMILES: Cc1ccccc1C(=O)NC(Cc1ccccc1)C(=O)Nc1ccccc1N1CCNCC1 ; O=C(NC(Cc1ccccc1)C(=O)Nc1ccccc1N1CCNCC1)c1cccs1 ; O=C(NC(Cc1ccccc1)C(=O)Nc1cc(F)ccc1N1CCNCC1)c1ccccc1F ; O=C(N[C@@H](Cc1ccccc1)C(=O)Nc1cc(F)ccc1N1CCNCC1)c1ccccc1F ; Cc1ccccc1C(=O)NC(Cc1ccccc1)C(=O)Nc1cc(F)ccc1N1CCNCC1 ; O=C(NC(Cc1ccccc1)C(=O)Nc1cc(F)ccc1N1CCNCC1)c1cccs1]

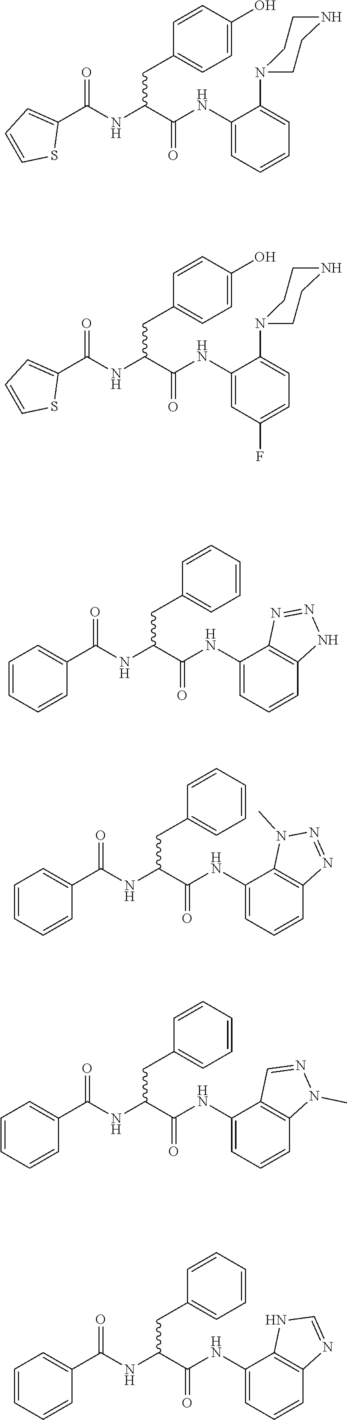 [SMILES: O=C(NC(Cc1ccc(O)cc1)C(=O)Nc1ccccc1N1CCNCC1)c1cccs1 ; O=C(NC(Cc1ccc(O)cc1)C(=O)Nc1cc(F)ccc1N1CCNCC1)c1cccs1 ; O=C(NC(Cc1ccccc1)C(=O)Nc1cccc2[nH]nnc12)c1ccccc1 ; Cn1nnc2cccc(NC(=O)C(Cc3ccccc3)NC(=O)c3ccccc3)c21 ; Cn1ncc2c(NC(=O)C(Cc3ccccc3)NC(=O)c3ccccc3)cccc21 ; O=C(NC(Cc1ccccc1)C(=O)Nc1cccc2nc[nH]c12)c1ccccc1]

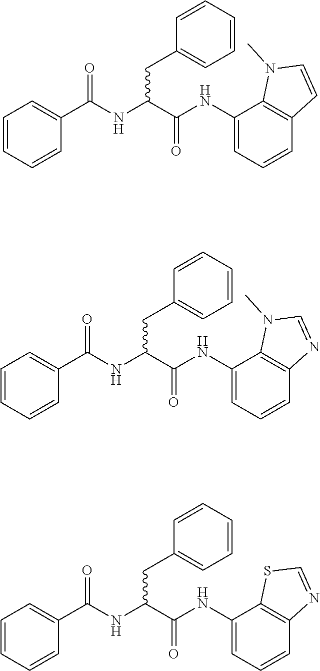 [SMILES: Cn1ccc2cccc(NC(=O)C(Cc3ccccc3)NC(=O)c3ccccc3)c21 ; Cn1cnc2cccc(NC(=O)C(Cc3ccccc3)NC(=O)c3ccccc3)c21 ; O=C(NC(Cc1ccccc1)C(=O)Nc1cccc2ncsc12)c1ccccc1]

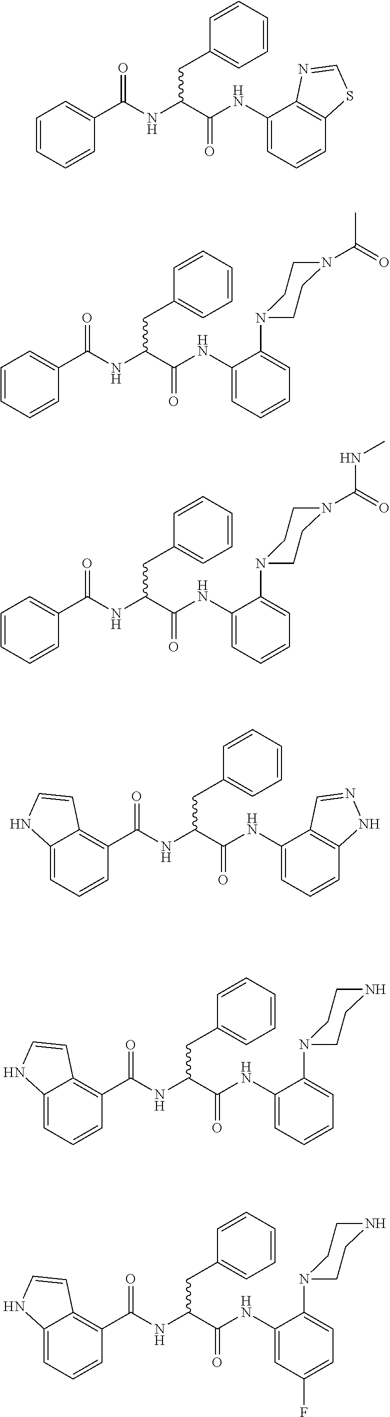 [SMILES: O=C(NC(Cc1ccccc1)C(=O)Nc1cccc2scnc12)c1ccccc1 ; CC(=O)N1CCN(c2ccccc2NC(=O)C(Cc2ccccc2)NC(=O)c2ccccc2)CC1 ; CNC(=O)N1CCN(c2ccccc2NC(=O)C(Cc2ccccc2)NC(=O)c2ccccc2)CC1 ; O=C(NC(Cc1ccccc1)C(=O)Nc1cccc2[nH]ncc12)c1cccc2[nH]ccc12 ; O=C(NC(Cc1ccccc1)C(=O)Nc1ccccc1N1CCNCC1)c1cccc2[nH]ccc12 ; O=C(NC(Cc1ccccc1)C(=O)Nc1cc(F)ccc1N1CCNCC1)c1cccc2[nH]ccc12]

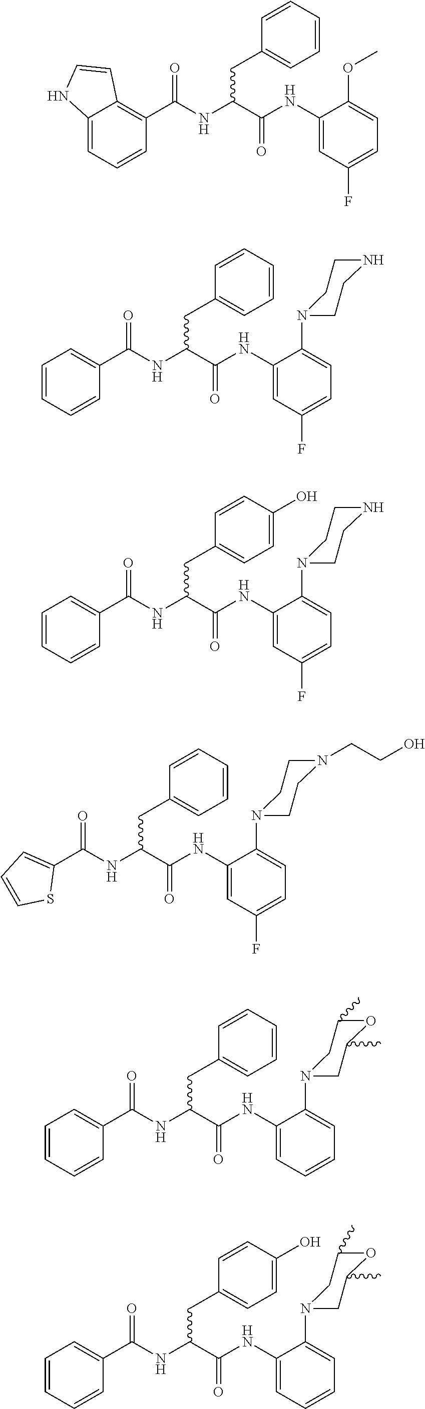

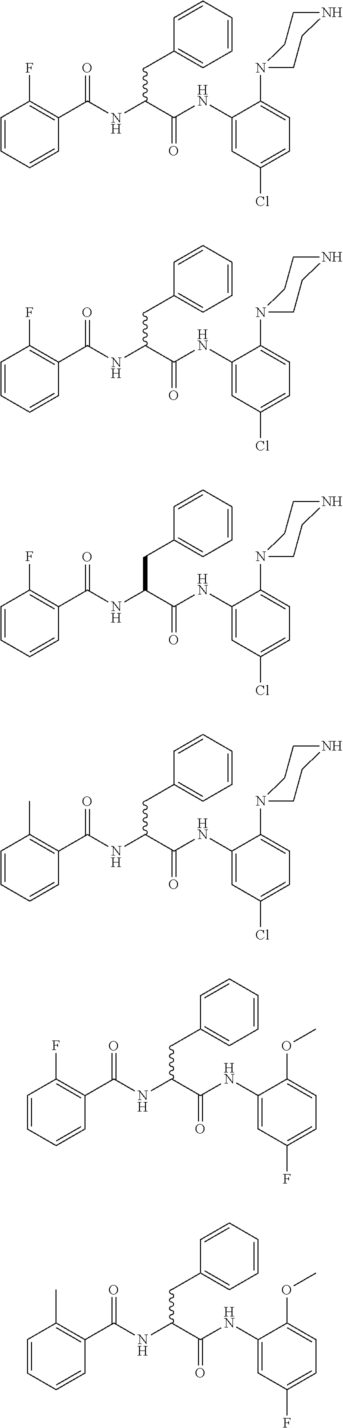 [SMILES: O=C(NC(Cc1ccccc1)C(=O)Nc1cc(Cl)ccc1N1CCNCC1)c1ccccc1F ; O=C(NC(Cc1ccccc1)C(=O)Nc1cc(Cl)ccc1N1CCNCC1)c1ccccc1F ; O=C(N[C@@H](Cc1ccccc1)C(=O)Nc1cc(Cl)ccc1N1CCNCC1)c1ccccc1F ; Cc1ccccc1C(=O)NC(Cc1ccccc1)C(=O)Nc1cc(Cl)ccc1N1CCNCC1 ; COc1ccc(F)cc1NC(=O)C(Cc1ccccc1)NC(=O)c1ccccc1F ; COc1ccc(F)cc1NC(=O)C(Cc1ccccc1)NC(=O)c1ccccc1C]

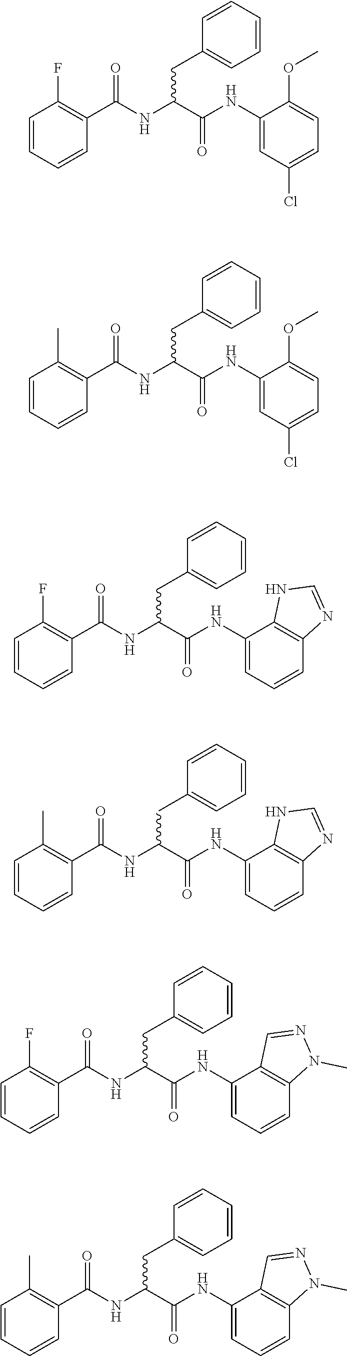 [SMILES: COc1ccc(Cl)cc1NC(=O)C(Cc1ccccc1)NC(=O)c1ccccc1F ; COc1ccc(Cl)cc1NC(=O)C(Cc1ccccc1)NC(=O)c1ccccc1C ; O=C(NC(Cc1ccccc1)C(=O)Nc1cccc2nc[nH]c12)c1ccccc1F ; Cc1ccccc1C(=O)NC(Cc1ccccc1)C(=O)Nc1cccc2nc[nH]c12 ; Cn1ncc2c(NC(=O)C(Cc3ccccc3)NC(=O)c3ccccc3F)cccc21 ; Cc1ccccc1C(=O)NC(Cc1ccccc1)C(=O)Nc1cccc2c1cnn2C]

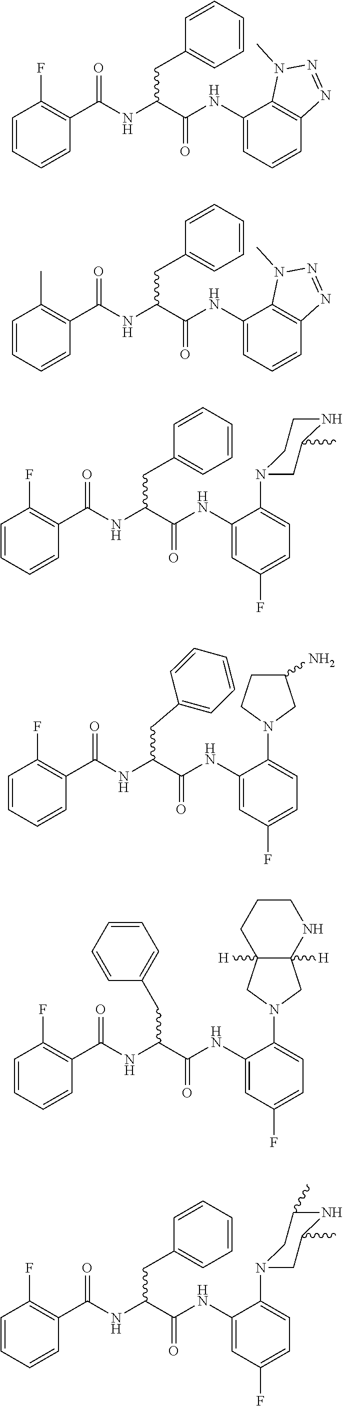

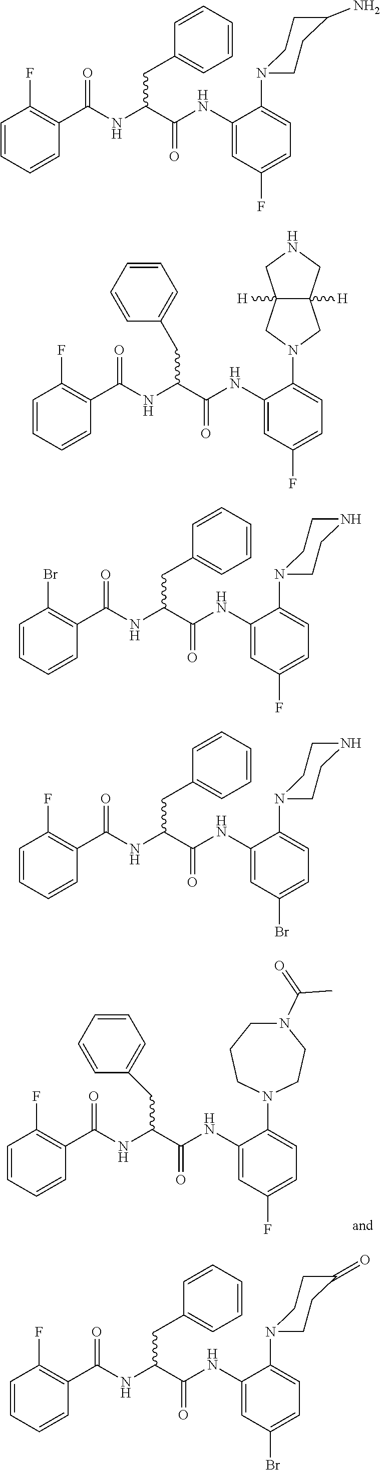

### Linker (β)

In one embodiment β is a linker that links the α moiety and the γ moiety. The linker preferably has a length of from about 0 Å to about 15 Å (representing a length suitable to connect α and γ.

The linker may comprise a covalent bond or multiple covalent bonds. Alternatively, the linker may comprise a coordinate-covalent bond.

Preferably, the linker does not substantially interfere with the individual interactions between the α moiety and the Rif target of a bacterial RNAP and between the γ moiety and the bridge-helix N-terminus target of a bacterial RNA polymerase.

Preferably, the linker does not substantially interfere with simultaneous interactions between the α moiety and the Rif target of a bacterial RNAP and between the γ moiety and the bridge-helix N-terminus target of a bacterial RNA polymerase.

Optionally, the linker makes a favorable interaction with at least one residue of RNAP located between the Rif target and the bridge-helix N-terminus target of a bacterial RNA polymerase.

In certain embodiments, β is a covalent bond.

In certain embodiments, β is two covalent bonds.

In certain embodiments, β comprises a chain of 0 to about 12 consecutively bonded atoms.

In certain embodiments, β comprises a chain of 0 to about 10 consecutively bonded atoms.

In certain embodiments, β comprises a chain of 0 to about 8 consecutively bonded atoms.

In certain embodiments, β comprises a chain of 0 to about 6 consecutively bonded atoms.

In certain embodiments, β comprises a chain of 1 to about 12 consecutively bonded atoms.

In certain embodiments, β comprises a chain of 1 to about 10 consecutively bonded atoms.

In certain embodiments, β comprises a chain of 1 to about 8 consecutively bonded atoms.

In certain embodiments, β comprises a chain of 1 to about 6 consecutively bonded atoms.

In one embodiment, the invention provides a compound selected from the group consisting of:

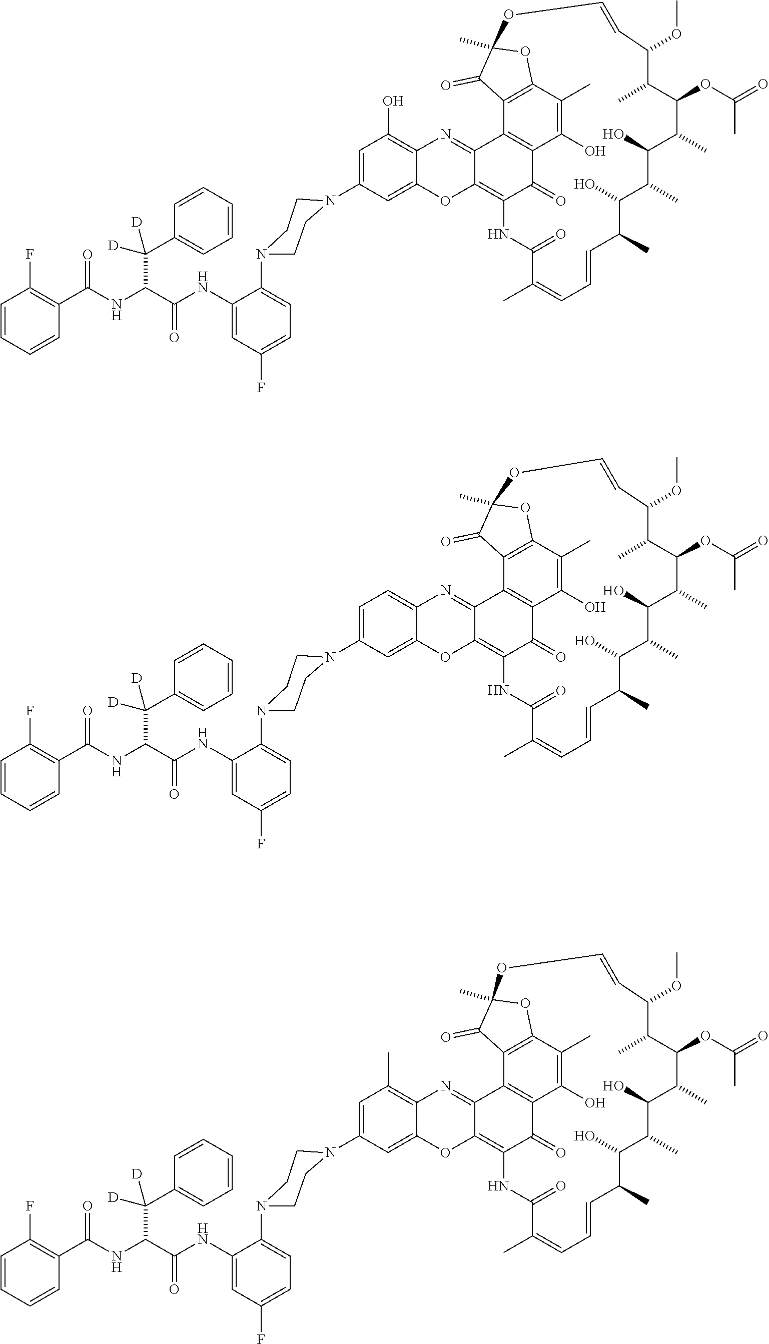

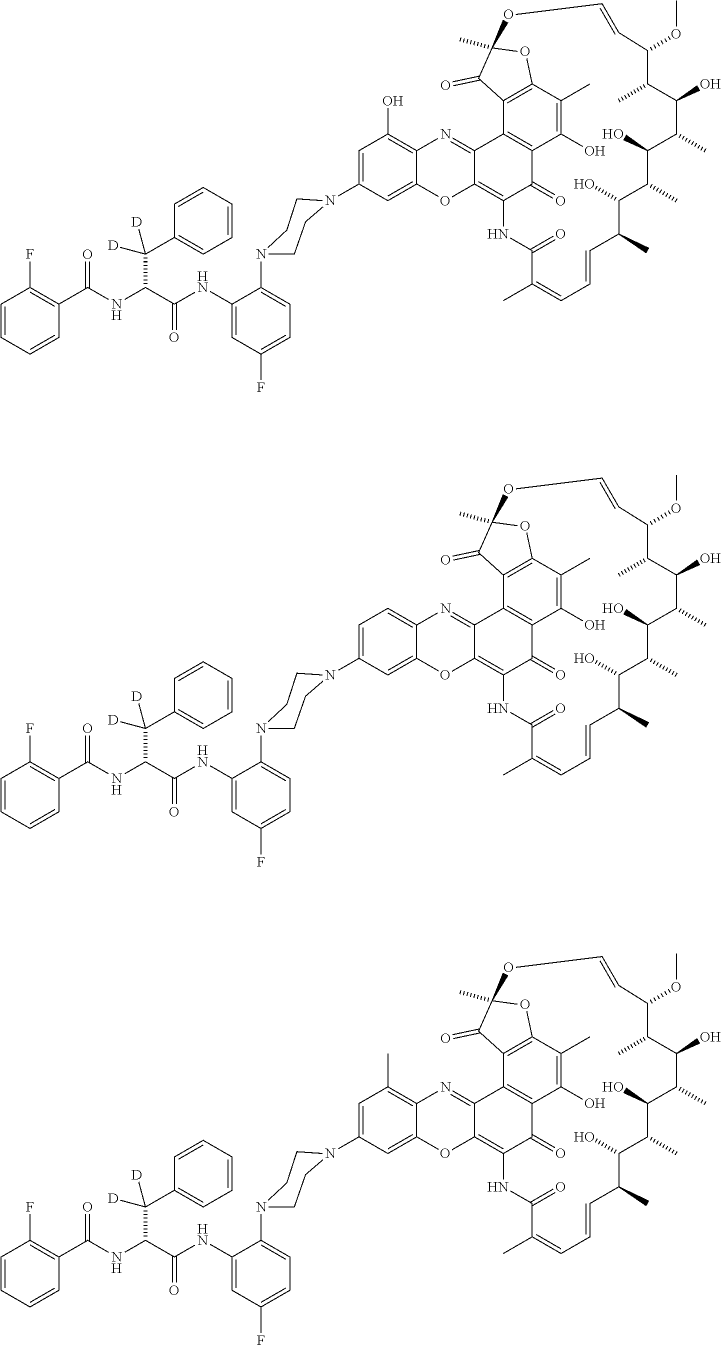

or a tautomer or salt thereof.

In one embodiment, the invention provides a compound selected from the group consisting of:

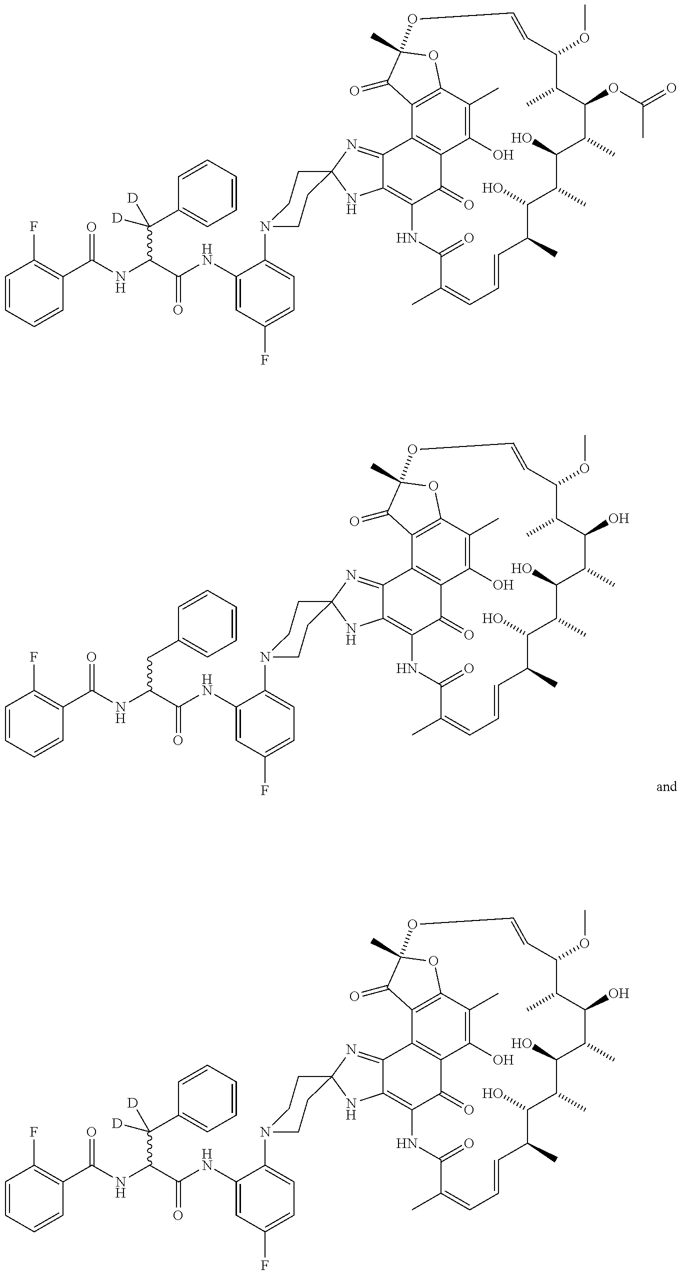

or a tautomer or salt thereof.

### Uses and Methods of Use of Dual-Targeted Inhibitors of RNAP

The invention provides bipartite inhibitors that interacts alternatively with the Rif target and the bridge-helix N-terminus target of a bacterial RNA polymerase; and therefore that typically exhibit at least one of the following useful characteristics:
(i) more potent inhibition of a bacterial RNAP than the individual α and the individual γ;
(ii) more potent antibacterial activity than the individual α and the individual γ;
(iii) potent inhibition of a bacterial RNAP resistant to one of the first RNAP inhibitor α and the second RNAP inhibitor γ; and
(iv) potent antibacterial activity against a bacterium resistant to one of the first RNAP inhibitor α and the second RNAP inhibitor γ.

This invention provides a compound comprising a first RNAP inhibitor that functions through the Rif target coupled to a second RNAP inhibitor that functions through the bridge-helix N-terminus target of a bacterial RNA polymerase.

In certain embodiments, a compound of the invention binds to a bacterial RNAP.

In certain embodiments, a compound of the invention binds to a bacterial RNAP resistant to at least one of α and γ.

In certain embodiments, a compound of the invention inhibits a bacterial RNAP.

In certain embodiments, a compound of the invention inhibits a bacterial RNAP with a potency higher than the potency of a and the potency of γ.

In certain embodiments of the invention, a compound of the invention inhibits a bacterial RNAP resistant to at least one of α and γ.

In certain embodiments of the invention, a compound of the invention inhibits bacterial growth. In certain embodiments, a compound of the invention inhibits bacterial growth with potencies higher than the potency of α and the potency of γ.

Certain embodiments provide the use a compound of the invention to bind to a bacterial RNAP.

Certain embodiments provide the use of a compound of the invention to inhibit a bacterial RNAP.

Certain embodiments provide the use of a compound of the invention to inhibit bacterial gene expression.

Certain embodiments provide the use of a compound of the invention to inhibit bacterial growth.

Certain embodiments provide the use of a compound of the invention to treat a bacterial infection.

Certain embodiments provide a composition comprising a compound of the invention or a pharmaceutically acceptable salt thereof, and a pharmaceutically acceptable vehicle.

Certain embodiments provide a method for inhibiting the growth of bacteria comprising contacting the bacteria with a compound of the invention, or a salt thereof.

Certain embodiments provide a method for inhibiting a bacterial RNAP comprising contacting the bacterial RNAP with a compound of the invention, or a salt thereof.

Certain embodiments provide a method for treating a bacterial infection in a mammal, e.g., a human, comprising administering to the mammal an effective amount of a compound of the invention, or a pharmaceutically acceptable salt thereof.

Certain embodiments provide a compound of formula (I), or a pharmaceutically acceptable salt thereof, for use in the prophylactic or therapeutic treatment of a bacterial infection.

Certain embodiments provide the use of a compound of formula (I), or a pharmaceutically acceptable salt thereof, for the preparation of a medicament for treating a bacterial infection in a mammal, e.g., a human.

In certain embodiments, the targeted bacterial species is selected from Gram-negative bacterial species, including for example, Escherichia coli (ECOLI), Salmonella typhimurium (SALTY), Klebsiella pneumoniae (KLEP7), Enterococcus cloacae (ENTCC), Vibrio cholerae (VIBCH), Haemophilus influenzae (HAEIN), Neisseria gonorrhoeae (NEIG1), Stenotrophomonas maltophilia (STPMP), Moraxella catarrhalis (MORCA), Acinetobacter baumannii (ACIBC), and Pseudomonas aeruginosa (PSEAE) (FIG. 3). In certain embodiments, the targeted bacterial species is selected from Mycobacteria, including, for example, Mycobacterium tuberculosis (MYCTU), Mycobacterium bovis, Mycobacterium avium (MYCA1), Mycobacterium abscessus (MYCA9), Mycobacterium abscessus, Mycobacterium chelonae, Mycobacterium fortuitum, Mycobacterium leprae, Mycobacterium ulcerans, and Mycobacterium smegmatis (MYCSM).

In certain embodiments, the targeted bacterial species is selected from a non-Mycobacterial Gram-positive bacterial species, including, for example, Staphylococcus aureus (STAAU), Staphylococcus epidermidis (STAEQ), Enterococcus faecalis (ENTFA), Streptococcus pyogenes (STRP1), Streptococcus pneumoniae (STRP2), and Clostridium difficile (CDIFF)).

### Methods of Preparing Dual-Targeted Inhibitors of RNAP

The invention also provides a method of preparing a compound having a structural formula (I):

α-β-γ (I)

wherein α is a spiro-rifamycin or a benzoxazino-rifamycin, γ comprises a moiety that binds to the bridge-helix N-terminus target of a bacterial RNA polymerase, and β is a linker. The method includes providing precursors α-B′ and ′β-γ, and reacting moieties β′ and ′β to form β. The precursors may include any suitable precursors that will bind to form a linker moiety and permit the α moiety to bind to the Rif target of the RNAP and permit the γ moiety to bind to the bridge-helix N-terminus target of a bacterial RNA polymerase.

For example, in a preferred embodiment, one precursor contains an aldehyde, a ketone, a protected aldehyde, or a protected ketone, and the other precursor contains a hydrazide or an amine. In another preferred embodiment, one precursor contains an activated ester, an imidazolide, or an anhydride and the other precursor contains an amine. In another preferred embodiment, one precursor contains a halogen and the other precursor contains an amine. In another preferred embodiment, one precursor contains a halogen and the other precursor contains a sulfhydryl. In another preferred embodiment, one precursor contains an azide and the other precursor contains an alkyne. In another preferred embodiment, one precursor contains an azide and the other precursor contains a phosphine. In another preferred embodiment, one precursor contains a boronic acid and the other precursor contains a substituted phenol. In another preferred embodiment, one precursor contains phenylboronic acid and the other precursor contains salicylhydroxamic acid.

Each of the above-referenced chemistries are established and are known to those skilled in the art (see Rostovetsev, et al. (2002) Angew. Chem. Int. Ed. 41, 2596-2599 Wang, et al. (2003) J. Amer. Chem. Soc. 125, 3192-3193; Breibauer, et al. (2003) ChemBioChem. 4, 1147-1149; Saxon, et al. (2000) Science 287, 2007-2010; Kiick, et al. (2002), Proc. Natl. Acad. Sci. USA 99, 19-24; Kohn, et al. (2004) Angew. Chem. Int. Ed. 43, 3106-3116; Stolowitz, et al. (2001) Bioconj. Chem. 12, 229-239; Wiley, et al. (2001), 12, 240-250).

In one embodiment, moieties α′ and ′α of precursors α-β′ and ′β-γ are reacted in the absence of a bacterial RNAP.

In another embodiment, moieties β′ and ′β of precursors α-β′ and ′β-γ are reacted in the presence of a bacterial RNAP. In this embodiment, the bacterial RNAP potentially can serve as a template for reaction of α-β′ and ′β-γ.

Certain embodiments of the invention provide a method of making a compound of the invention, wherein the compound is prepared from precursors α-β′ and ′β-γ, wherein β′ and ′β are moieties that can react to form β.

In certain embodiments, one precursor contains an aldehyde, a ketone, a protected aldehyde, or a protected ketone, and the other precursor contains a hydrazide or an amine.

In certain embodiments, one precursor contains an activated ester, an imidazolide, or an anhydride, and the other precursor contains an amine.

In certain embodiments, one precursor contains a haloacetyl moiety, and the other precursor contains an amine.

In certain embodiments, one precursor contains a halogen, and the other precursor contains an amine.

In certain embodiments, one precursor contains a haloacetyl moiety, and the other precursor contains a sulfhydryl.

In certain embodiments, one precursor contains a halogen, and the other precursor contains a sulfhydryl.

In certain embodiments, one precursor contains an azide, and the other precursor contains an alkyne.

In certain embodiments, one precursor contains an azide, and the other precursor contains a phosphine.

In certain embodiments, one precursor contains a boronic acid, and the other precursor contains a substituted phenol.

In certain embodiments, one precursor contains phenylboronic acid, and the other precursor contains salicylhydroxamic acid.

In certain embodiments, precursors α-β′ and ′β-γ are allowed to react in the absence of a bacterial RNAP.

In certain embodiments, precursors α-β′ and ′β-γ are allowed to react in the presence of a bacterial RNAP.

In certain embodiments, the bacterial RNAP serves as a template for reaction of α-β′ and ′β-γ.

### Pharmaceutical Preparations and Methods of Administration

In cases where compounds are sufficiently basic or acidic, a salt of a compound of the invention can be useful as an intermediate for isolating or purifying a compound of the invention. Additionally, administration of a compound of the invention as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartarate, succinate, benzoate, ascorbate, α-ketoglutarate, and α-glycerophosphate. Suitable inorganic salts may also be formed, including hydrochloride, sulfate, nitrate, bicarbonate, and carbonate salts.

Pharmaceutically acceptable salts may be obtained using standard procedures well known in the art, for example by reacting a sufficiently basic compound such as an amine with a suitable acid affording a physiologically acceptable anion. Alkali metal (for example, sodium, potassium or lithium) or alkaline earth metal (for example calcium) salts of carboxylic acids can also be made.

The compound of the invention can be formulated as pharmaceutical compositions and administered to a mammalian host, such as a human patient in a variety of forms adapted to the chosen route of administration, i.e., orally or parenterally, by intravenous, intramuscular, topical, or subcutaneous routes.

Thus, the present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Examples of useful dermatological compositions which can be used to deliver the compound of the invention to the skin are known to the art; for example, see Jacquet et al. (U.S. Pat. No. 4,608,392), Geria (U.S. Pat. No. 4,992,478), Smith et al. (U.S. Pat. No. 4,559,157) and Wortzman (U.S. Pat. No. 4,820,508).

Useful dosages of the compound of the invention can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.5 to about 150 mg/kg, e.g., from about 10 to about 100 mg/kg of body weight per day, such as 3 to about 75 mg per kilogram body weight of the recipient per day, preferably in the range of 6 to 120 mg/kg/day, most preferably in the range of 15 to 90 mg/kg/day.

The compound is conveniently formulated in unit dosage form; for example, containing 5 to 1000 mg, conveniently 10 to 750 mg, most conveniently, 50 to 500 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

The present invention is not to be limited in scope by the specific embodiments describe herein. Indeed, various modifications of the invention in addition to those described herein will become apparent to those skilled in the art from the foregoing description and the accompanying figures. Such modifications are intended to fall within the scope of the appended claims.

### INDUSTRIAL APPLICABILITY

Compounds identified according to the target and method of this invention would have applications not only in antibacterial therapy, but also in: (a) identification of bacterial RNAP (diagnostics, environmental-monitoring, and sensors applications), (b) labeling of bacterial RNAP (diagnostics, environmental-monitoring, imaging, and sensors applications), (c) immobilization of bacterial RNAP (diagnostics, environmental-monitoring, and sensors applications), (d) purification of bacterial RNA polymerase (biotechnology applications), (e) regulation of bacterial gene expression (biotechnology applications), and (f) antisepsis (antiseptics, disinfectants, and advanced-materials applications).

The invention will now be illustrated by the following non-limiting examples.

### Examples

With reference to the examples below, Applicant has identified compounds that inhibit bacterial RNAP and inibit bacterial growth.

### Example 1: Synthesis of 3'-hydroxy-benzoxazinorifamycin S-(IX-370a) conjugate (IX-511a)

### Example 1.1: Synthesis of 2-amino-3-tert-butyl-dimethylsilyloxyphenol

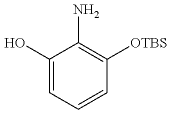 [SMILES: CC(C)(C)[Si](C)(C)Oc1cccc(O)c1N]

2-amino-3-tert-butyl-dimethylsilyloxyphenol was synthesized by a modification of the procedure of Yamane et al. (Chem. Pharm. Bull. 41 (1) 148-155, 1993). 2-amino-1,3-benzenediol (0.108 mg; 0.863 mmol; Sigma-Aldrich) and imidazole (0.147 mg; 2.16 mmol; Sigma-Aldrich) were dissolved in 2 ml anhydrous DMF. Tert-butyl-dimethylchlorosilane (156 mg; 1.04 mmol; Sigma-Aldrich) in 1 ml anhydrous DMF was added to the reaction dropwise over 30 minutes. The reaction was stirred for another 10 minutes, quenched with 3 ml saturated ammonium chloride, and extracted with 3×3 ml ethyl acetate. The organic extracts were pooled, washed with brine, dried over anhydrous sodium sulfate, filtered, evaporated, and purified by silica chromatography (ethyl acetate/hexanes gradient). Yield: 100 mg; 50%. MS (MALDI): calculated: m/z 240.35 (M+H⁺); found: 240.33.

### Example 1.2: Synthesis of 3′-(tert-butyldimethylsilyl)oxy)-benzoxazinorifamycin S

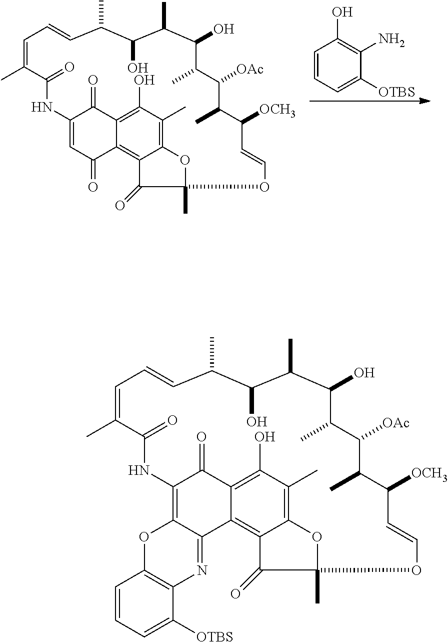 [SMILES: CO[C@H]1/C=C/O[C@@]2(C)Oc3c(C)c(O)c4c(c3C2=O)C(=O)C=C(NC(=O)/C(C)=C\C=C\[C@H](C)[C@H](O)[C@@H](C)[C@@H](O)[C@@H](C)[C@H](OC(C)=O)[C@@H]1C)C4=O ; CC(C)(C)[Si](C)(C)Oc1cccc(O)c1N ; CO[C@H]1/C=C/O[C@@]2(C)Oc3c(C)c(O)c4c(=O)c(c5oc6cccc(O[Si](C)(C)C(C)(C)C)c6nc-5c4c3C2=O)NC(=O)/C(C)=C\C=C\[C@H](C)[C@H](O)[C@@H](C)[C@@H](O)[C@@H](C)[C@H](OC(C)=O)[C@@H]1C]

Rifamycin S (44 mg; 0.063 mmol; AvaChem Scientific) and 2-amino-3-tert-butyl-dimethylsilyloxyphenol (Example 1.1) were stirred together in 0.8 ml toluene for 16 h and then evaporated to dryness. To the residue, was added 0.6 ml anhydrous ethanol and manganese dioxide (25 mg; 0.29 mmol; Sigma-Aldrich). The suspension was stirred for 30 min, filtered with a Celite pad, evaporated to dryness, and purified by silica chromatography (ethyl acetate/hexanes gradient). Yield: 40 mg; 70%. MS (MALDI): calculated: m/z 916.12 (M+H+); found: 916.20.

### Example 1.3: Synthesis of (R)-2-fluoro-N-(1-((5-fluoro-2-(piperazin-1-yl)phenyl)amino)-1-oxo-3-phenylpropan-2-yl-3,3-d₂) benzamide (IX-370a)

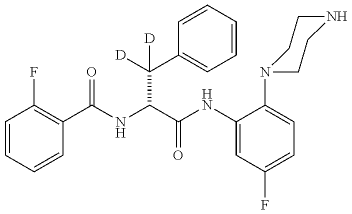 [SMILES: [2H]C([2H])(c1ccccc1)[C@@H](NC(=O)c1ccccc1F)C(=O)Nc1cc(F)ccc1N1CCNCC1]

(R)-2-fluoro-N-(1-((5-fluoro-2-(piperazin-1-yl)phenyl)amino)-1-oxo-3-phenylpropan-2-yl-3,3-d₂) benzamide was synthesized as in Ebright, R., Ebright, Y., Mandal, S., Wilde, R., and Li, S. (2018) Antibacterial agents: N (alpha)-aroyl-N-aryl-phenylalaninamides. U.S. Pat. No. 9,919,998. Yield: 2.9 g; 25.6% (overall yield, starting from 2,5-difluoronitrobenzene). MS (MALDI): calculated: m/z 666.79 (M+H⁺); found: 667.25.

### Example 1.4: Synthesis of 3-hydroxy-benzoxazinorifamycin S-(IX-370a) conjugate (IX-511a)

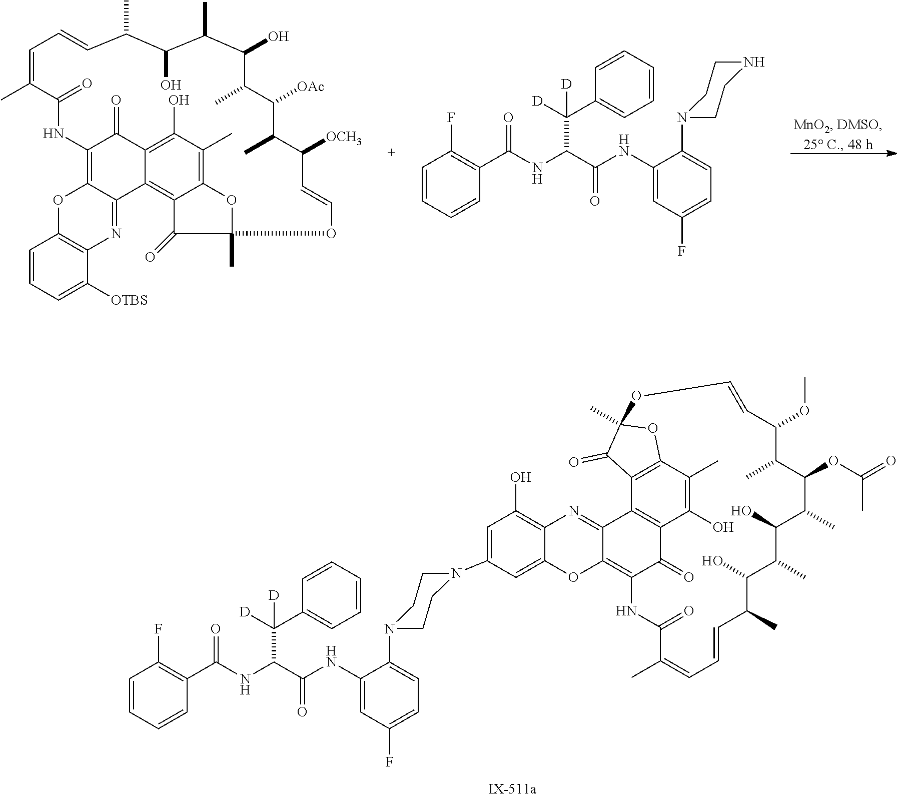 [SMILES: CO[C@H]1/C=C/O[C@@]2(C)Oc3c(C)c(O)c4c(=O)c(c5oc6cccc(O[Si](C)(C)C(C)(C)C)c6nc-5c4c3C2=O)NC(=O)/C(C)=C\C=C\[C@H](C)[C@H](O)[C@@H](C)[C@@H](O)[C@@H](C)[C@H](OC(C)=O)[C@@H]1C ; [2H]C([2H])(c1ccccc1)[C@@H](NC(=O)c1ccccc1F)C(=O)Nc1cc(F)ccc1N1CCNCC1 ; [2H]C([2H])(c1ccccc1)[C@@H](NC(=O)c1ccccc1F)C(=O)Nc1cc(F)ccc1N1CCN(c2cc(O)c3nc4c5c6c7c(C)c(O)c5c(=O)c(c-4oc3c2)NC(=O)/C(C)=C\C=C\[C@H](C)[C@H](O)[C@@H](C)[C@@H](O)[C@@H](C)[C@H](OC(C)=O)[C@H](C)[C@@H](OC)/C=C/O[C@@](C)(O7)C6=O)CC1]

To a solution of 3′-(tert-butyldimethylsilyl)oxy)-benzoxazinorifamycin (60 mg; 0.066 mmol; Example 1.2) in 3 ml DMSO, was added IX370a (60 mg; 0.13 mmol; example 1.3) and manganese dioxide (60 mg; 0.69 mmol; Sigma-Aldrich). The suspension was stirred at 25° C. for 60 h, after which 24 ml ethyl acetate was added. The suspension was filtered through a Celite pad. The filtrate was washed with 20 ml water, 20 ml brine, dried over anhydrous sodium sulfate, evaporated, and purified by silica chromatography (ethyl acetate/hexanes gradient). Yield: 10.5 mg; 13%. MS (MALDI): calculated: m/z 1266.35 (M+H⁺); found: 1266.49 (M+H⁺), 1287.46 (M+Na⁺), 1234.49 (M-MeOH).

### Example 2: Synthesis of benzoxazinorifamycin S-(IX-370a) conjugate (IX-516a)

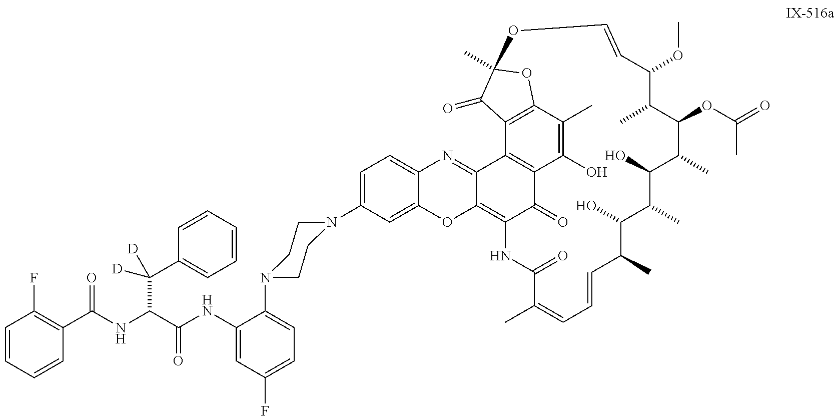 [SMILES: [2H]C([2H])(c1ccccc1)[C@@H](NC(=O)c1ccccc1F)C(=O)Nc1cc(F)ccc1N1CCN(c2ccc3nc4c5c6c7c(C)c(O)c5c(=O)c(c-4oc3c2)NC(=O)/C(C)=C\C=C\[C@H](C)[C@H](O)[C@@H](C)[C@@H](O)[C@@H](C)[C@H](OC(C)=O)[C@H](C)[C@@H](OC)/C=C/O[C@@](C)(O7)C6=O)CC1]

IX-516a was prepared as described for IX-511a in Example 1, but using 2-aminophenol (Sigma-Aldrich) in place of 2-amino-3-tert-butyl-dimethylsilyloxyphenol. Yield: 12.44 mg; 19.5%. MS (MALDI): calculated: m/z 1250.32 (M+H⁺); found: 1249.68, 1272.65 (M+Na⁺), 1218.74 (M-MeOH).

### Example 3: Synthesis of 3′-methyl-benzoxazinorifamycin S-(IX-370a) conjugate (IX-517a)

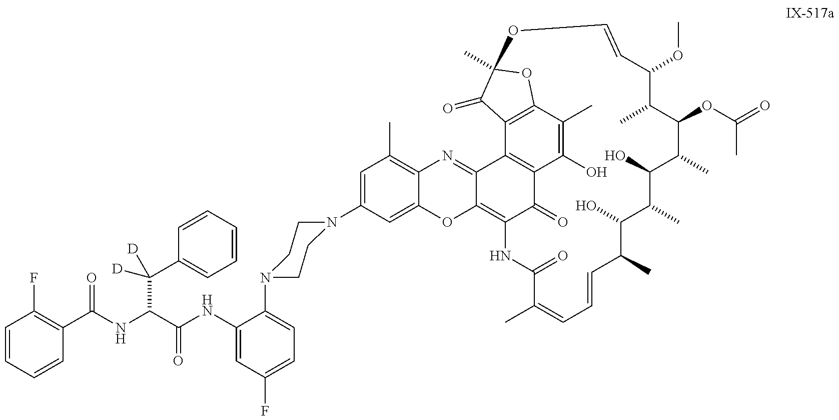 [SMILES: [2H]C([2H])(c1ccccc1)[C@@H](NC(=O)c1ccccc1F)C(=O)Nc1cc(F)ccc1N1CCN(c2cc(C)c3nc4c5c6c7c(C)c(O)c5c(=O)c(c-4oc3c2)NC(=O)/C(C)=C\C=C\[C@H](C)[C@H](O)[C@@H](C)[C@@H](O)[C@@H](C)[C@H](OC(C)=O)[C@H](C)[C@@H](OC)/C=C/O[C@@](C)(O7)C6=O)CC1]

IX-517a was prepared as described for IX-511a in Example 1, but using 2-amino-m-cresol (Sigma-Aldrich) in place of 2-amino-3-tert-butyl-dimethylsilyloxyphenol. Yield: 17 mg; 16%. MS (MALDI): calculated: m/z 1264.40 (M+H+); found: 1264.40, 1286.40 (M+Na⁺), 1231.40 (M-MeOH).

### Example 4: Synthesis of spirorifamycin S-(IX-513) conjugate (IX-515)

### Example 4.1: Synthesis of 2-fluoro-N-(1-((5-fluoro-2-(4-oxopiperadin-1-yl)phenyl) amino)-1-oxo-3-phenylpropan-2-yl-3,3-d₂) benzamide (LX-513)

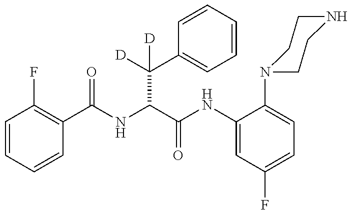 [SMILES: [2H]C([2H])(c1ccccc1)[C@@H](NC(=O)c1ccccc1F)C(=O)Nc1cc(F)ccc1N1CCNCC1]

IX-513 was prepared as described for IX-370a (Example 1.3), but using 4-piperidone ethylene acetal (Sigma-Aldrich) in place of 1-Boc-piperazine. The resulting acetal was hydrolysed with HCl to give IX-513. Yield: 114 mg; 38.4% (overall yield, starting from 2,5-difluoronitrobenzene). MS (MALDI): calculated: m/z 479.52 (M+H⁺); found: 480.25.

### Example 4.2: Synthesis of spirorifamycin S-(IX-513) conjufate (IX-515)

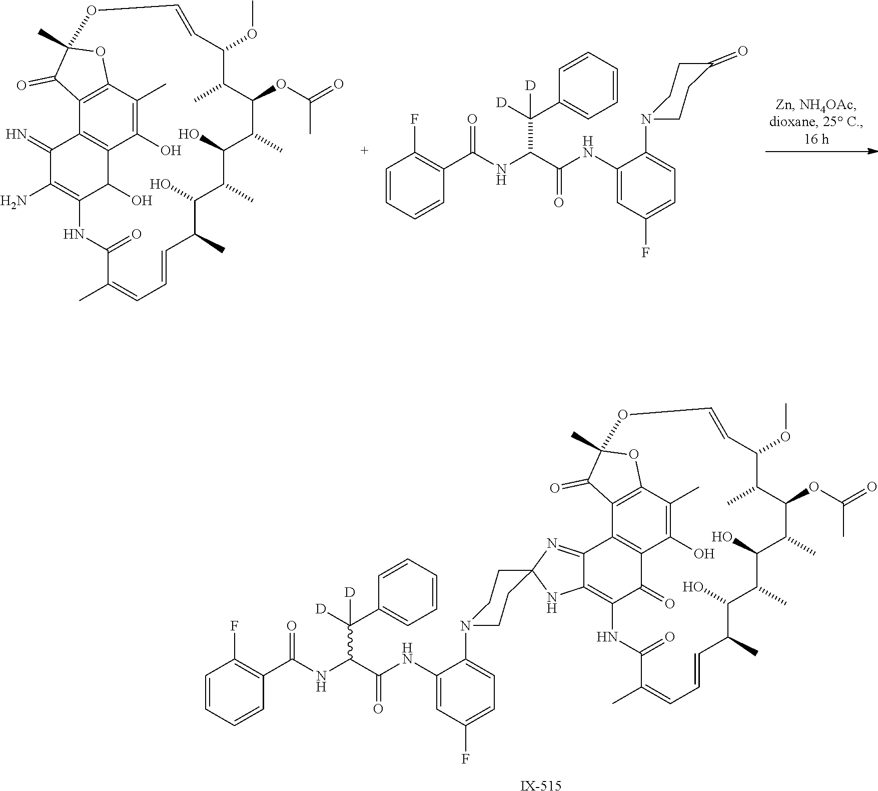

3-amino-4-imino-Rif S (40 mg; 0.056 mmol; BOC Sci), ammonium acetate (5 mg, 0.06 mmol; Sigma-Aldrich), zinc dust (5 mg, 0.08 mmol; Sigma-Aldrich), and IX-513 (Example 4.1) were stirred together in 0.2 ml anhydrous dioxane for 16 h. The reaction mixture then was centrifuged at 8,000×g for 10 min, and the supernatant was collected and added to 10 ml ethyl acetate. The solution was washed with 2 ml water, washed with 2 ml brine, dried over anhydrous sodium sulfate, filtered, evaporated, and purified by silica chromatography (ethyl acetate/hexanes gradient). Yield: 30 mg; 45% yield. MS (MALDI): calculated: m/z 1172.3 (M+H⁺); found: 1193.60 (M+Na⁺), 1141.7 (M-MeOH).

### Example 5: Synthesis of desacetyl-3′-hydroxy-benzoxazinorifamycin S-(IX-370a) conjugate (IX-519a)

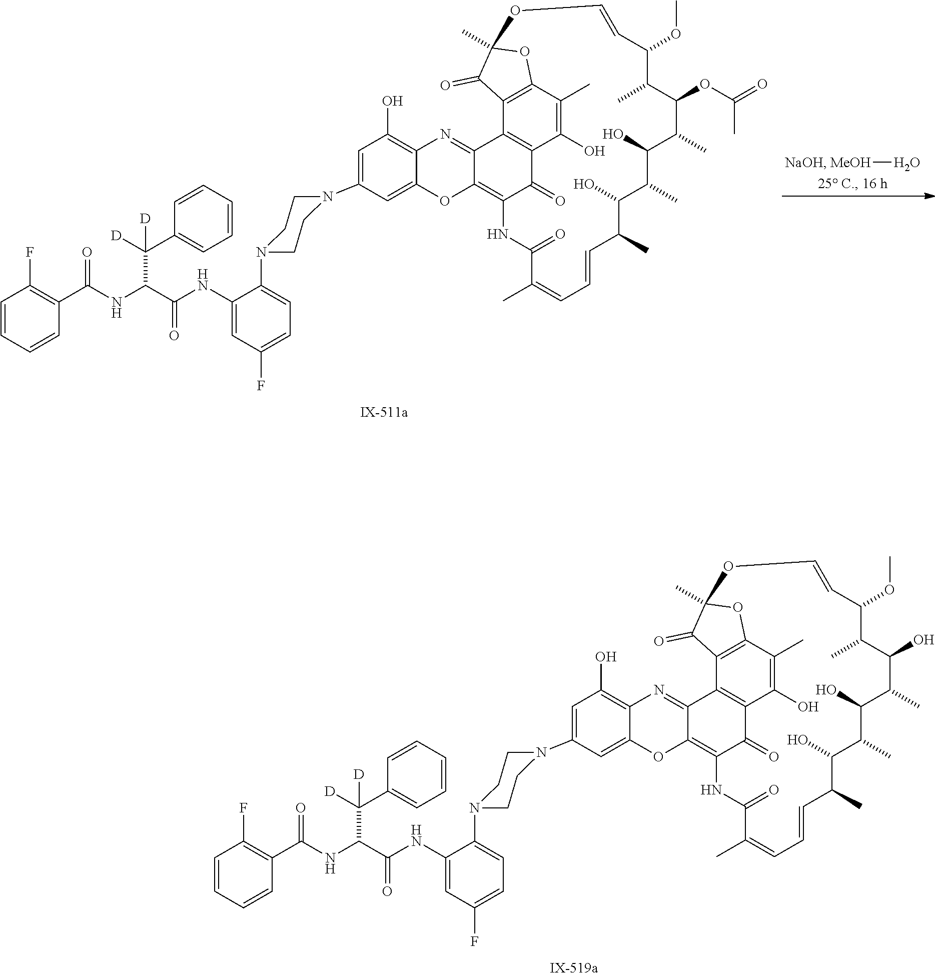

To IX-511a (100 mg in 5 mL methanol, 0.079 mmol), was added 0.80 mmol sodium hydroxide (4 ml 0.2 M solution in 1:1 MeOH: water) and 0.1 mmol zinc chloride (1 ml freshly prepared 0.1 M solution in water). The reaction was stirred at 25° C. for 16 h. The reaction mixture was quenched with 30 ml ice water, was extracted with 2×30 ml ethyl acetate, and the pooled ethyl acetate extacts were dried over anhydrous sodium sulfate. The product was purified by silica chromatography (ethyl acetate/hexanes gradient). Yield: 75 mg; 77% yield. MS (MALDI): calculated: m/z 1224.35 (M+H+); found: 1246.56 (M+Na⁺), 1192.57 (M-MeOH).

### Example 6: Synthesis of desacetyl-spirorifamycin S-(IX-513) conjugate (IX-520)

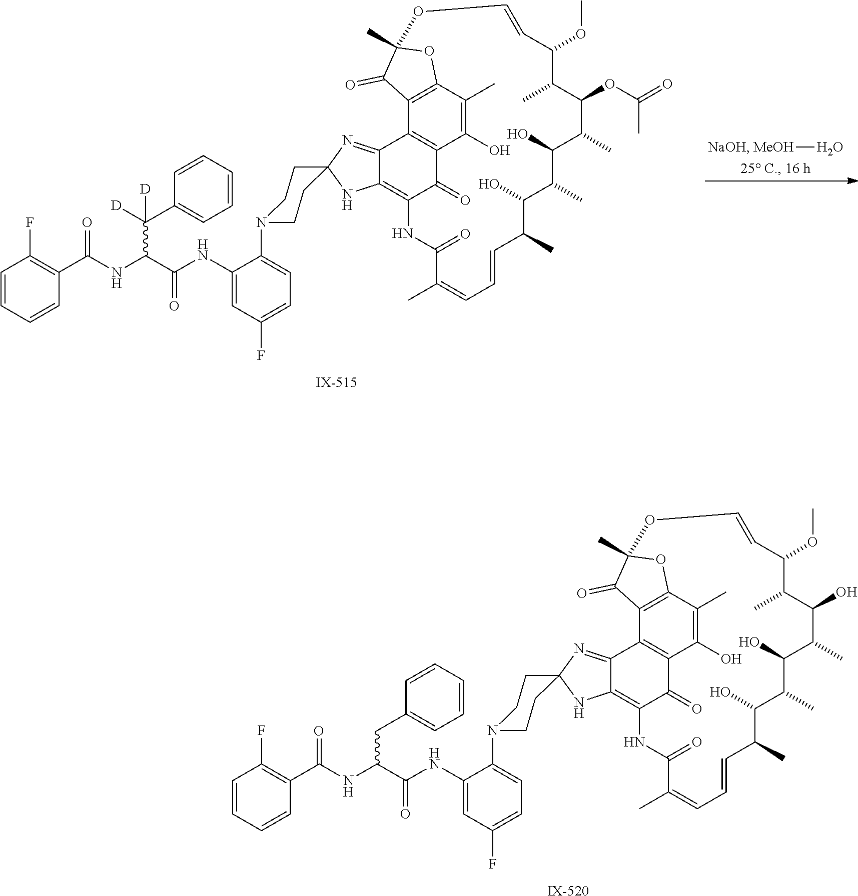

To IX-515 (100 mg in 5 ml methanol, 0.085 mmol), was added 0.85 mmol sodium hydroxide (4.25 ml 0.2 M solution in 1:1 MeOH-water) and 0.1 mmol zinc chloride (1 ml freshly prepared 0.1 M solution in water). The reaction was stirred at 25° C. for 16 hours. The reaction mixture was quenched with 30 ml ice water, was extracted with 2×30 ml ethyl acetate, and the pooled ethyl acetate extracts were dried over anhydrous sodium sulfate. The product was purified by silica chromatography (ethyl acetate/hexanes gradient). The product was found to have undergone hydrogen exchange, replacing the two deuterium atoms for two hydrogen atoms. Yield: 81.5 mg; 85% yield. MS (MALDI): calculated: m/z 1128.25 (M+H⁺); found: 1128.65, 1097.73 (M-MeOH).

### Example 7: Synthesis of deuterated desacetyl-spirorifamycin S-(IX-513) conjugate (IX-520D)

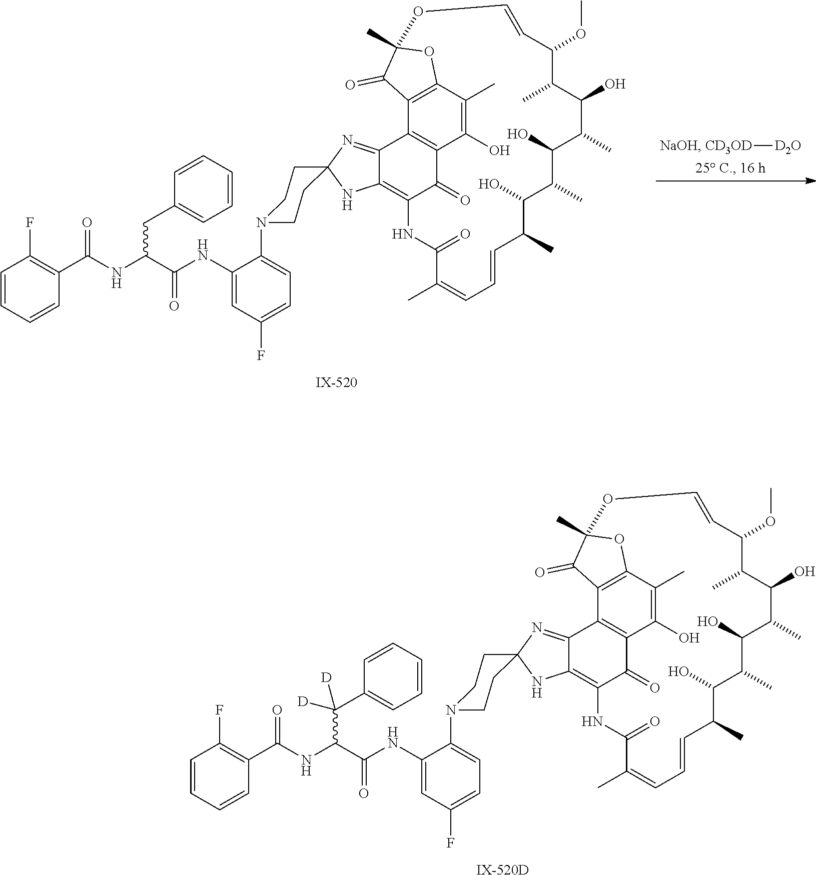

To IX-520 (0.6 mg in 30 ul CD₃OD methanol, 0.5 umol), was added 5 umol sodium hydroxide [25 ul 0.2 M solution in 1:1 CD₃OD (Sigma-Aldrich): D₂O (Sigma-Aldrich)] and 0.6 umol zinc chloride (6 ul freshly prepared 0.1 M solution in D₂O). The reaction was stirred at 25° C. for 16 h. The reaction mixture was quenched with 1 ml ice water, was extracted with 2×1 ml ethyl acetate, and the pooled ethyl acetate extracts were dried over anhydrous sodium sulfate. Yield: 0.5 mg; 88% crude yield. MS (MALDI): calculated: m/z 1130.25 (M+H⁺); found: 1130.64, 1052.59 (M+Na⁺), 1098.75 (M-MeO-).

### Example 8: Synthesis of desacetyl-3′-benzoxazinorifamycin S-(IX-370a) conjugate (IX-521a)

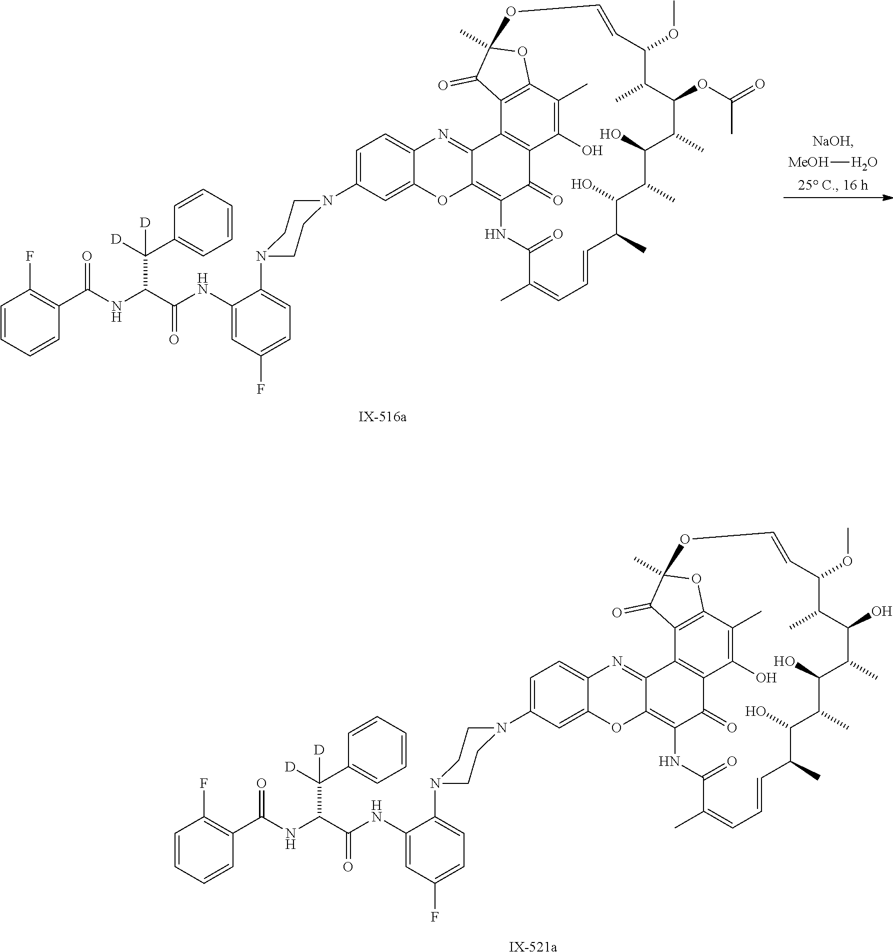

To IX-516a (50 mg in 5 ml methanol, 0.040 mmol), was added 0.080 mmol sodium hydroxide (2 ml 0.2 M solution in 1:1 MeOH-water) and 0.048 mmol zinc chloride (0.48 ml freshly prepared 0.1 M solution in water). The reaction was stirred at 25° C. for 16 h. The reaction mixture was quenched with 15 ml ice water, was extracted with 2×15 ml ethyl acetate, and the pooled ethyl acetate extracts were dried over anhydrous sodium sulfate. The product was purified by silica chromatography (ethyl acetate/hexanes gradient). Yield: 35 mg; 73% yield. MS (MALDI): calculated: m/z 1208.34 (M+H⁺); found: 1230.59 (M+Na⁺), 1176.58 (M-MeOH).

### Example 9: Synthesis of desacetyl-3′-methyl-benzoxazinorifamycin S (IX-370a) conjugate (IX-522a)

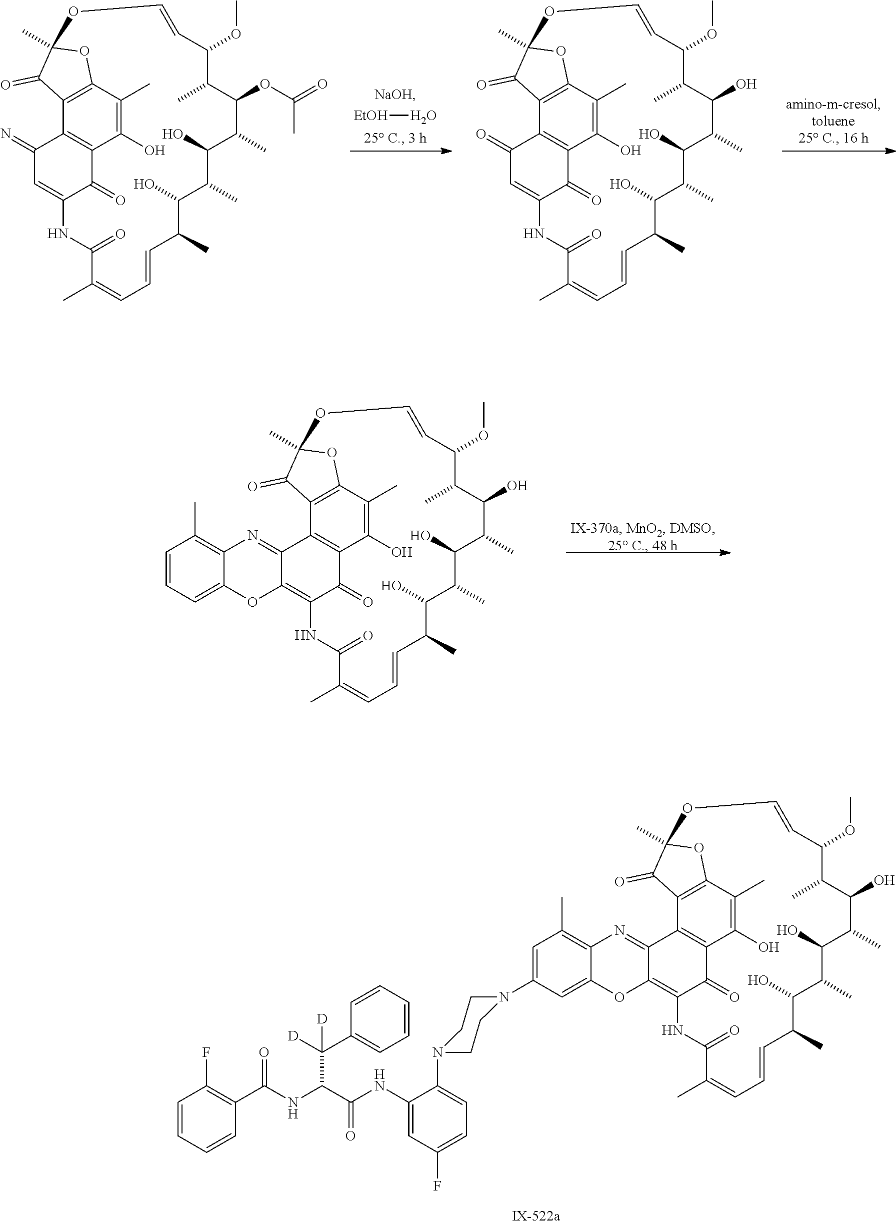

IX-522a was synthesized by an alternative method. Rifamycin S was desacetylated to provide O-25-desacetyl rifamycin S, according to the method of Maggi and Sensi, 1980 [Maggi, N. and Sensi, P. 25-Desacetyl rifamycins. U.S. Pat. No. 4,188,321 (1980)]. The resulting provide desacetyl-rifamycin S was reacted with amino-cresol to provide desacetyl-3′-methyl-benzoxazino-rifamycin S, which then was reacted with IX-370a to provide desacetyl-3′-methyl-benzoxazinorifamycin S-(IX-370a) conjugate (IX-522a).

### Example 9.1: Synthesis of desacetyl-rifamycin S

To rifamycin S (44 mg, 0.063 mmol; AvaChem Scientific), was added 3.15 mL 0.5% ethanolic sodium hydroxide (0.5 g sodium hydroxide dissolved in 5 ml water, followed by adding 95 ml ethanol). The resulting red-violet solution was stirred at 25° C. for 3 h. 10 ml ice water was added, and the pH was adjusted to 4 by dropwise addition of 1M HCl, resulting in precipitation of a yellow solid. The reaction mizture was extracted with 2×10 ml ethyl acetate, and the pooled ethyl acetate extracts were dried over anhydrous sodium sulfate. The product was purified by silica chromatography (ethyl acetate/hexanes gradient). TLC in ethyl acetate showed a spot with reduced Rf compared with rifamycin S. Yield: 38 mg; 92% yield.

### Example 9.2: Synthesis of desacetyl-3′-methyl-benzoxazinorifamycin S

To desacetyl-rifamyin S (38 mg, 0.058 mmol; Example 9.1) in 1 ml toluene, was added 2-amino-m-cresol (8 mg, 0.062 mmol, Sigma-Aldrich). The reaction was stirred at 25° C. for 16 h and then was evaporated to dryness. To the residue, was added 1 ml anhydrous ethanol and manganese dioxide (25 mg; 0.29 mmol; Sigma-Aldrich). The suspension was stirred for 30 min, filtered with a Celite pad, evaporated to dryness, and purified by silica chromatography (ethyl acetate/hexanes gradient). Yield: 31 mg; 71%. MS (MALDI): calculated: m/z 757.86 (M+H⁺); found 779.41 (M+Na⁺), 725.42 (M-MeOH).

### Example 9.3: Synthesis of desacetyl-3′-methyl-benzoxazinorifamycin S-(IX-370a) conjugate (IX-522a)

IX-522a was prepared as described for IX-511a in Example 1.4, but using 3′-methyl-benzoxazino-desacetyl-rifamycin S (Example 9.2) in place of 3′-(tert-butyldimethylsilyl)oxy)-benzoxazinorifamycin S. Yield: 8.2 mg; 26%. MS (MALDI): calculated: m/z 1222.36 (M+H⁺); found: 1243.56 (M+Na⁺), 1189.56 (M-MeOH).

### Example 10: Assay of RNAP-Inhibitory Activity

Fluorescence-Detected RNA Polymerase Assays were Performed by a Modification of the procedure of Kuhlman et al., 2004 [Kuhlman, P., Duff, H. and Galant, A. (2004) A fluorescence-based assay for multisubunit DNA-dependent RNA polymerases. Anal. Biochem. 324, 183-190]. Reaction mixtures contained (20 μL): 0-100 nM test compound, 75 nM Mycobacterium tuberculosis RNA polymerase core enzyme or Mycobacterium tuberculosis RNA polymerase core enzyme derivative [prepared as in Lin, W., Mandal, S., Degen, D., Liu, Y., Ebright, Y. W., Li, S., Feng, Y., Zhang, Y., Mandal, S., Jiang, Y., Liu, S., Gigliotti, M., Talaue, M., Connell, N., Das, K., Arnold, E., and Ebright, R. H. (2017) Mol. Cell 66, 169-179], 300 nM Mycobacterium. tuberculosis σ^{A}, 20 nM 384 bp DNA fragment containing the bacteriophage T4 N25 promoter, 100μ M ATP, 100μ M GTP, 100μ M UTP, 100μ M CTP, 40 mM Tris-HCl, pH 8.0, 80 mM NaCl, 5 mM MgCl₂, 2.5 mM DTT, and 12.7% glycerol. Reaction components other than DNA and NTPs were pre-incubated for 10 minutes at 37° C. Reactions were carried out by addition of DNA and incubation for 5 minutes at 37° C., followed by addition of NTPs and incubation for 60 minutes at 37° C. DNA was removed by addition of 1 μL 5 mM CaCl₂) and 2 U DNaseI (Ambion, Inc.), followed by incubation for 90 minutes at 37° C. RNA was quantified by addition of 100 μl RiboGreen RNA Quantitation Reagent (Invitrogen, Inc.; 1:500 dilution in Tris-HCl, pH 8.0, 1 mM EDTA), followed by incubation for 10 minutes at 25° C., followed by measurement of fluorescence intensity [excitation wavelength=485 nm and emission wavelength=535 nm; QuantaMaster QM1 spectrofluorimeter (PTI, Inc.)]. IC₅₀ is defined as the concentration of inhibitor resulting in 50% inhibition of RNA polymerase activity.

Data for compounds of this invention and the comparator compounds rifampin and IX-370a are presented in Table 1.

TABLE 1
Inhibition of bacterial RNAP.

| | | RNAP-inhibitory | RNAP-inhibitory |
|---|---|---|---|
| | | activity | activity |
| | | rifampin-resistant | AAP-resistant |
| | RNAP-inhibitory | M. tuberculosis | M. tuberculosis |
| | activity | RNAP (rpoB- | RNAP (rpoB- |
| | M. tuberculosis | S′531′L) | R′637′C) |
| compound | RNAP IC50 (μM) | IC50 (μM) | IC50 (μM) |
| rifampin | 0.03 | >400 | 0.04 |
| IX-370a | 0.9 | 0.3 | 5 |
| IX-511a | 0.03 | 2 | 0.006 |
| IX-515 | 0.02 | 2 | 0.01 |
| IX-516a | 0.01 | 1 | 0.004 |
| IX-517a | 0.01 | 5 | 0.01 |
| IX-519a | 0.05 | 64 | 0.03 |
| IX-520H | 0.05 | 0.1 | 0.2 |
| IX-522a | 0.1 | 35 | 0.05 |

The data in Table 1 show that certain compounds of this invention potently inhibit a bacterial RNA polymerase.

The data in Table 1 further show that certain compounds of this invention inhibit a rifampin-resistant bacterial RNA polymerase >6 to >4,000 times more potently than rifampin (underlined in table).

The data in Table 1 further show that certain compounds of this invention inhibit an AAP-resistant bacterial RNA polymerase 25 to 900 times more potently than IX-370a (italicized in table).

### Example 11: Assay of Antibacterial Activity

MICs for Mycobacterium tuberculosis H37Rv; rifampin-resistant Mycobacterium tuberculosis isolates 10571 (rpoB-D′516′V), 20626 (rpoB-H′526′D), 4457 (rpoB-H′526′Y), and 14571 (rpoB-S′531′L; and Mycobacterium avium ATCC 25291) were quantified using microplate Alamar Blue assays as described [Collins, L. and Franzblau, S. (1997) Antimicrob. Agents Chemother. 41, 1004-1009]. MICs for Mycobacterium abscessus IDR-1400012185 were quantified were quantified using broth microdilution assays as described [Clinical and Laboratory Standards Institute (CLSI/NCCLS) (2009) Methods for Dilution Antimicrobial Susceptibility Tests for Bacteria That Grow Aerobically; Approved Standard, Eighth Edition. CLIS Document M07-A8 (CLIS, Wayne PA)].

Data for compounds of this invention and the comparator compound rifampin are presented in Tables 2-3.

TABLE 2
Inhibition of bacterial growth, Mycobacterium tuberculosis.

| | | antibacterial | antibacterial | antibacterial | antibacterial |
|---|---|---|---|---|---|
| | | activity | activity | activity | activity |
| | | rifampin- | rifampin- | rifampin- | rifampin- |
| | antibacterial | resistant | resistant | resistant | resistant |
| | activity | M. | M. | M. | M. |
| | M. | tuberculosis | tuberculosis | tuberculosis | tuberculosis |
| | tuberculosis | 10571 (rpoB- | 20626 (rpoB- | 4457 (rpoB- | 10545 (rpoB- |
| | H37Rv | D′516′V) | H′526′D) | H′526′Y) | S′531′L) |
| compound | MIC (μg/ml) | MIC (μg/ml) | MIC (μg/ml) | MIC (μg/ml) | MIC (μg/ml) |
| rifampin | 0.063 | 12.5 | >50 | >50 | >50 |
| IX-511a | 0.0020 | 0.016 | 0.78 | 3.13 | 50 |
| IX-515 | 0.031 | 0.25 | >50 | 12.5 | >50 |
| IX-516a | 0.031 | 0.78 | >50 | >50 | >50 |
| IX-519a | 0.024 | 12.5 | 3.1 | 50 | 12.5 |
| IX-520 | 0.024 | 0.040 | 0.20 | 0.20 | 0.20 |

The data in Table 2 show that certain compounds of this invention potently inhibit growth of Mycobacterium tuberculosis.

The data in Table 2 further show that certain compounds of this invention inhibit a rifampin-resistant isolate of Mycobacterium tuberculosis 2 to >300 times more potentiy than rifampin (underlined in table).

TABLE 3
Inhibition of bacterial growth, non-
tuberculous Mycobacteria (NTM)

| | antibacterial activity | antibacterial activity |
|---|---|---|
| | M. avium | M. abscessus |
| compound | ATCC 25291 | IDR-1400012185 |
| rifampin | 0.024 | 1.56 |
| IX-511a | 0.024 | 1.56 |
| IX-515 | 0.78 | 6.25 |
| IX-516a | | 3.13 |
| IX-520 | | 0.39 |

The data in Table 3 show that certain compounds of this invention potently inhibit growth of the non-tubercular Mycobacteria (NTMs) Mycobacterium avium and Mycobacterium abscessus.

### Example 12: Assay of Cytochrome P450 Induction Activity

Imduction of cytochrome P450 3A4 (CYP3A4) activity in human hepatocytes (male, Caucasian) was assayed by multiple-reaction-montoring LC-MS-MS, using midazolam as CCYP3A4-specific substrate and 1′-hydroxymidazolam as CYP3A4-specific product, essentially as described [Rhodes, S., Otten, J., Hingorani, G., Hartley, D., Franklin, R. (2011) J. Pharmacol. Toxicol. Meths. 63, 223-226].

Data for compounds of this invention and the comparator compound rifampin are presented in Table 4.

TABLE 4
Cytochrome P450 induction

| | CYP3A4 induction activity |
|---|---|
| compound | IC50 (μM) |
| rifampin | <1 |
| IX-511a | 10 |
| IX-515 | >10 |
| IX-516a | 10 |

The data in Table 4 show that, in contrast to the comparator compound rifampin, certain compounds of this invention do not potently induce cytochrome P450 3A4 (CYP3A4), The data suggest that, in contrast to the comparator compound rifampin, certain compounds of this invention will not exhibit unfavorable drug interactions associated with induction of CYP3A4.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A compound of formula (I):
α-β-γ (I)
or a tautomer thereof, or salt thereof, wherein:
α is a benzoxazino-rifamycin or a spiro-rifamycin;
β is a bond, or two bonds, or a-linker comprising at least one atom and at least two bonds; and
γ is a moiety that binds to the bridge-helix N-terminus target of a bacterial RNA polymerase.

2. The compound, tautomer, or salt of claim 1, wherein α is a bednzoxazino-rifamycin.

3. The compound, tautomer, or salt of claims 1 and 2, wherein α is: 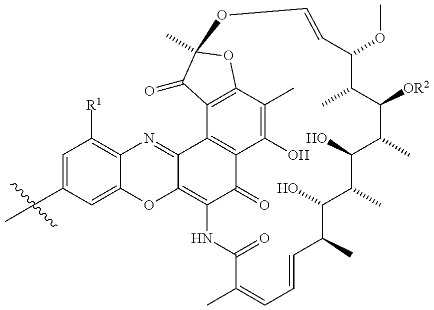
where R¹ is one of hydrogen, hydroxyl, and (C₁-C₆)alkyl, and R² is one of hydrogen, R^{x}, and —C(═O)NR^{a}R^{b}; wherein each R^{a} and R^{b} is one of hydrogen and (C₁-C₆) alkyl or R^{a} and R^{b} together with the nitrogen to which they are attached form a aziridino, azetidino, morpholino, piperazino, pyrrolidino or piperidino; and R^{x} is “(C₁-C₆) alkanoyl optionally substituted by halogen, aroyl optionally substituted by halogen, (C₃-C₅) heteroaroyl optionally substituted by halogen.

4. The compound, tautomer, or salt of claim 1, wherein α is a spiro-rifamycin.

5. The compound, tautomer, or salt of claim 1, wherein α is: 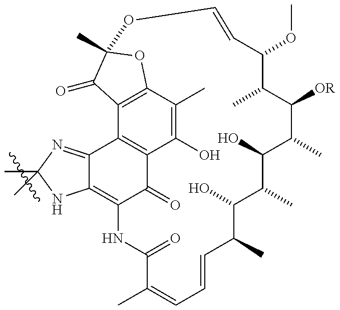
where R is one of hydrogen, R^{x}, and —C(═O) NR^{a}R^{b}, wherein each R^{a} and R^{b} is one of hydrogen and (C₁-C₆) alkyl or R^{a} and R^{b} together with the nitrogen to which they are attached form a aziridino, azetidino, morpholino, piperazino, pyrrolidino or piperidino; and R^{x} is “(C₁-C₆) alkanoyl optionally substituted by halogen, aroyl optionally substituted by halogen, (C₃-C₅) heteroaroyl optionally substituted by halogen.

6. The compound, tautomer, or salt of any one of claims 1-5, wherein γ has formula (II): 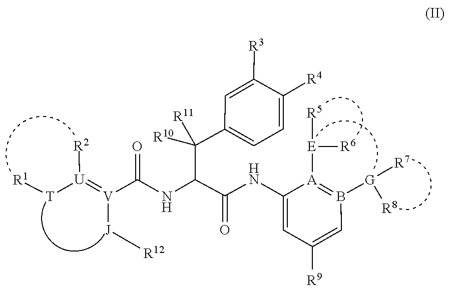
wherein:
T and U each is one of carbon and nitrogen;
E is carbon;
A and B each is one of carbon and nitrogen;
Y is one of carbon, nitrogen, oxygen, and sulfur;
Z is one of hydrogen, halogen, carbon, nitrogen, oxygen, and sulfur;
J is one of carbon and nitrogen, and J, together with T, U, and V forms part of a 6-membered cycle; or J is one of nitrogen, oxygen, sulfur, and selenium, and J, together with T, U, and V forms part of a 5-membered cycle;
R¹ and R² each independently is absent, hydrogen, hydroxy, or halogen, or is alkyl, alkoxy-substituted alkyl, amino-substituted alkyl, aryl-substituted alkyl, or alkoxy, each optionally substituted by halogen; or R¹ and R², together with T and U, form a cycle containing 4 to 9 atoms selected from carbon, nitrogen, oxygen, and sulfur;
R³ and R⁴ each independently is hydrogen, halogen, hydroxyl, amine, amide, ester, phosphate, or O-methylphosphate;
R⁵, R⁶, R⁷, and R⁸ each independently is absent, hydrogen, or halogen, or is alkyl, alkoxy-substituted alkyl, hydroxy-substituted alkyl, amino-substituted alkyl, aryl-substituted alkyl, or alkoxy, each optionally substituted by halogen; or R⁵ and R⁶, together with E, form a cycle containing 3 to 8 atoms selected from carbon, nitrogen, oxygen, and sulfur, said cycle optionally substituted with halogen, amino, alkyl, alkoxy-substituted alkyl, amino-substituted alkyl, aryl-substituted alkyl, alkoxy, acyl, or carbamidyl, each alkyl, alkoxy-substituted alkyl, amino-substituted alkyl, aryl-substituted alkyl, alkoxy, acyl, or carbamidyl optionally substituted by halogen; or R⁷ and R⁸, together with G, form a cycle containing 3 to 8 atoms selected from carbon, nitrogen, oxygen, and sulfur, said cycle optionally substituted with halogen, amino, alkyl, alkoxy-substituted alkyl, hydroxy-substituted alkyl, amino-substituted alkyl, or aryl-substituted alkyl, alkoxy, acyl, or carbamidyl, each alkyl, alkoxy-substituted alkyl, amino-substituted alkyl, aryl-substituted alkyl, alkoxy, acyl, or carbamidyl optionally substituted by halogen; or R⁶ and R⁷ are absent and E and G, together with A and B, form a cycle containing 4 to 9 atoms selected from carbon, nitrogen, oxygen, and sulfur, said cycle optionally substituted with halogen, amino, alkyl, alkoxy-substituted alkyl, amino-substituted alkyl, aryl-substituted alkyl, alkoxy, acyl, or carbamidyl, each alkyl, alkoxy-substituted alkyl, amino-substituted alkyl, aryl-substituted alkyl, alkoxy, acyl, or carbamidyl optionally substituted by halogen;
R⁹ is hydrogen or halogen;
R¹⁰ and R¹¹ each independently is one of hydrogen, halogen, alkyl, or alkoxy, said alkyl or alkoxy optionally substituted by halogen; or one of R¹⁰ and R¹¹ is deuterium, and the other is halogen, alkyl, or alkoxy, said alkyl or alkoxy optionally substituted by halogen; or each of R¹⁰ and R¹¹ is deuterium; and
R¹² is absent, hydrogen, or halogen;
or a tautomer or salt thereof.

7. The compound, tautomer, or salt of any one of claims 1-5, wherein γ has formula (IIa):

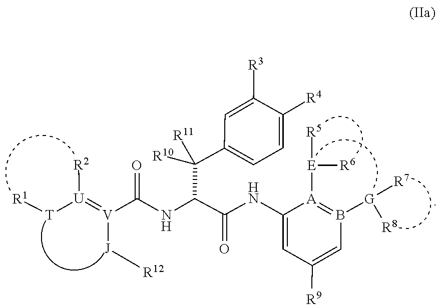

8. The compound, tautomer, or salt of any one of claims 1-5, wherein γ is selected from the group consisting of:

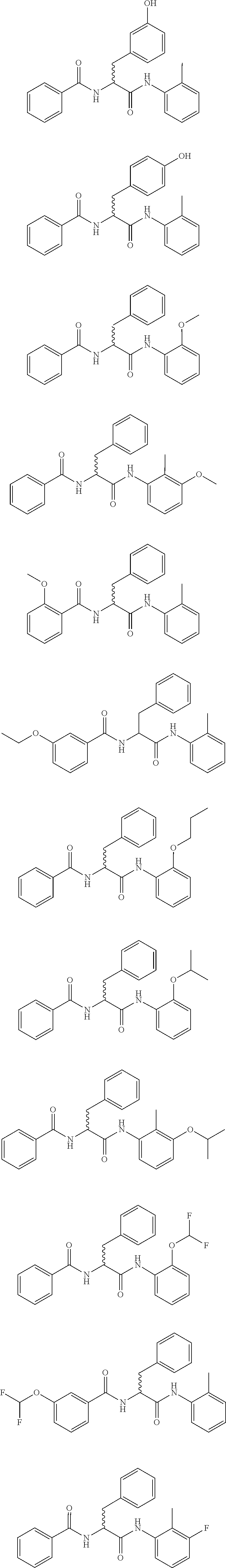

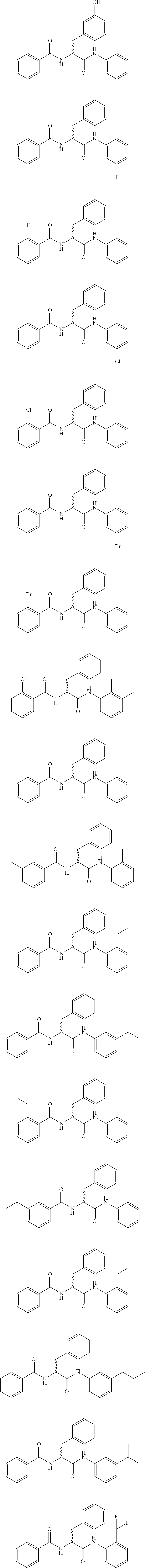

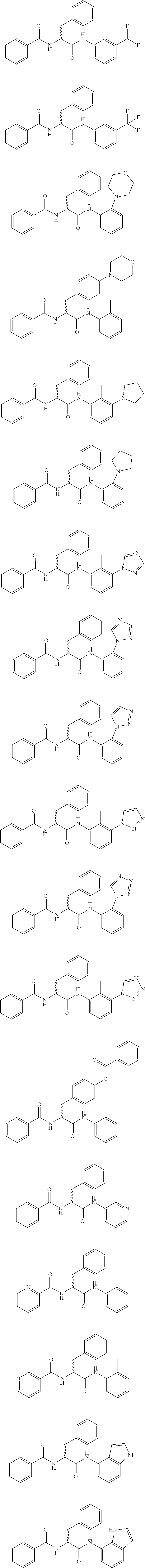

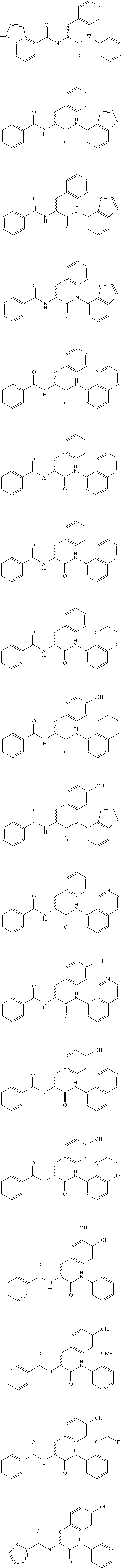

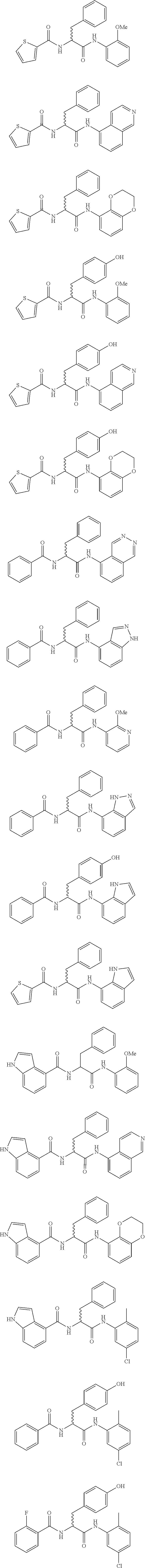

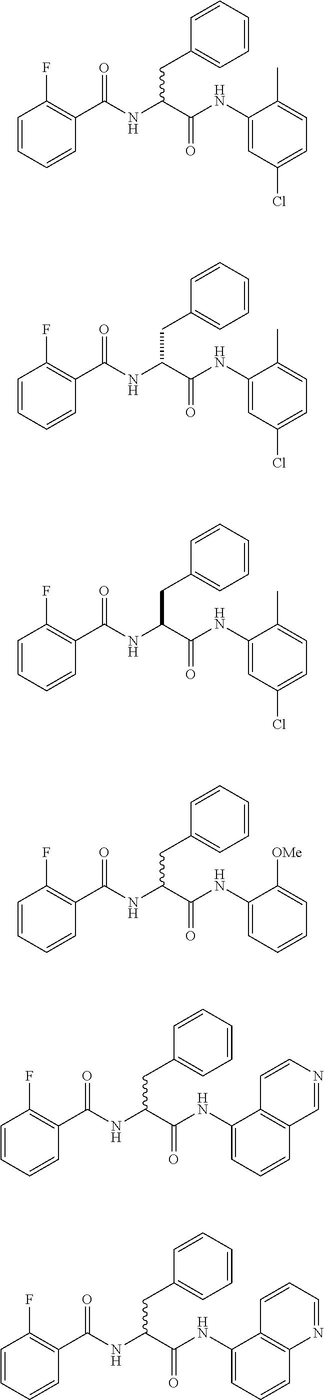 [SMILES: Cc1ccc(Cl)cc1NC(=O)C(Cc1ccccc1)NC(=O)c1ccccc1F ; Cc1ccc(Cl)cc1NC(=O)[C@@H](Cc1ccccc1)NC(=O)c1ccccc1F ; Cc1ccc(Cl)cc1NC(=O)[C@H](Cc1ccccc1)NC(=O)c1ccccc1F ; COc1ccccc1NC(=O)C(Cc1ccccc1)NC(=O)c1ccccc1F ; O=C(NC(Cc1ccccc1)C(=O)Nc1cccc2cnccc12)c1ccccc1F ; O=C(NC(Cc1ccccc1)C(=O)Nc1cccc2ncccc12)c1ccccc1F]

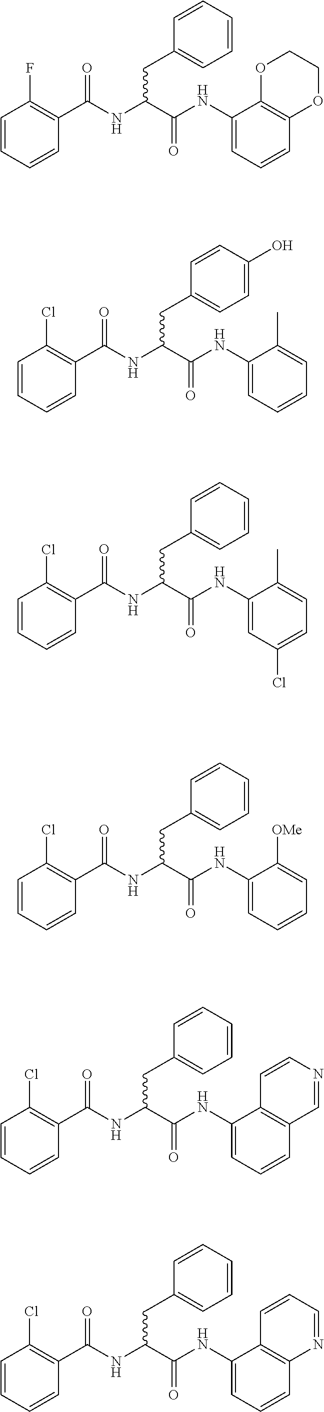 [SMILES: O=C(NC(Cc1ccccc1)C(=O)Nc1cccc2c1OCCO2)c1ccccc1F ; Cc1ccccc1NC(=O)C(Cc1ccc(O)cc1)NC(=O)c1ccccc1Cl ; Cc1ccc(Cl)cc1NC(=O)C(Cc1ccccc1)NC(=O)c1ccccc1Cl ; COc1ccccc1NC(=O)C(Cc1ccccc1)NC(=O)c1ccccc1Cl ; O=C(NC(Cc1ccccc1)C(=O)Nc1cccc2cnccc12)c1ccccc1Cl ; O=C(NC(Cc1ccccc1)C(=O)Nc1cccc2ncccc12)c1ccccc1Cl]

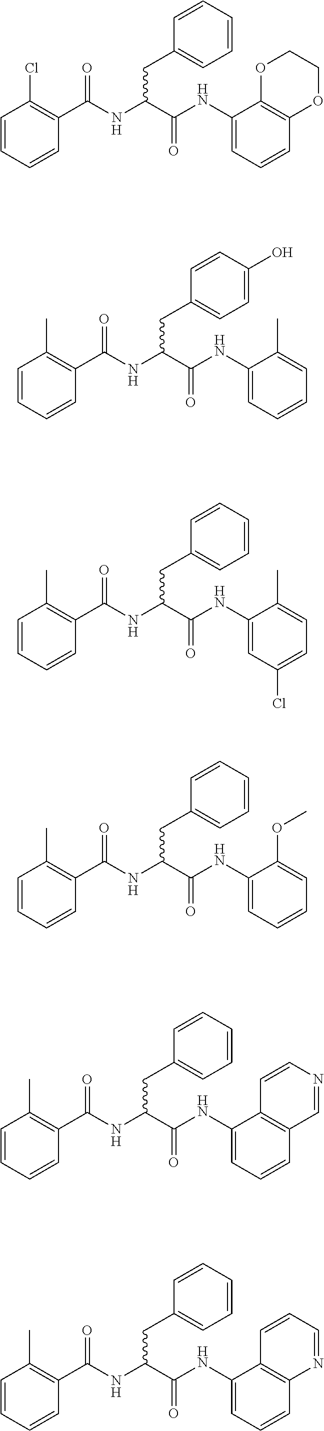 [SMILES: O=C(NC(Cc1ccccc1)C(=O)Nc1cccc2c1OCCO2)c1ccccc1Cl ; Cc1ccccc1NC(=O)C(Cc1ccc(O)cc1)NC(=O)c1ccccc1C ; Cc1ccc(Cl)cc1NC(=O)C(Cc1ccccc1)NC(=O)c1ccccc1C ; COc1ccccc1NC(=O)C(Cc1ccccc1)NC(=O)c1ccccc1C ; Cc1ccccc1C(=O)NC(Cc1ccccc1)C(=O)Nc1cccc2cnccc12 ; Cc1ccccc1C(=O)NC(Cc1ccccc1)C(=O)Nc1cccc2ncccc12]

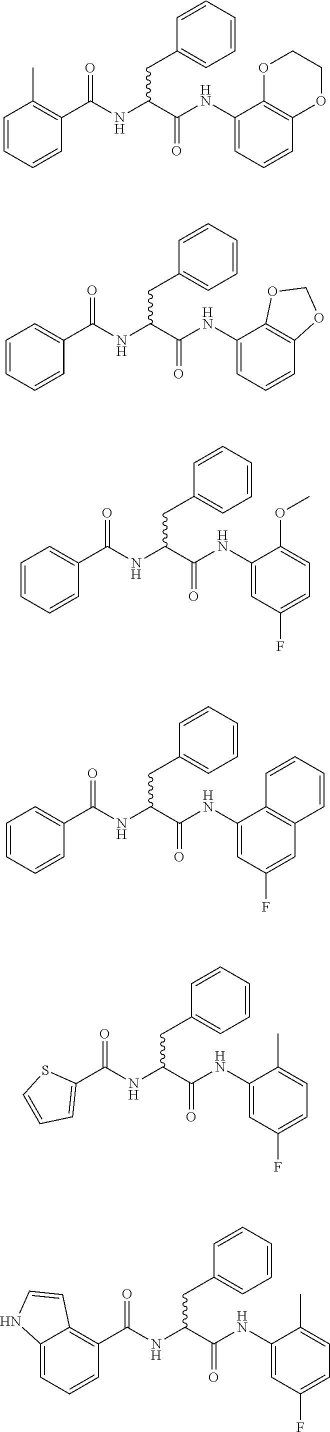 [SMILES: Cc1ccccc1C(=O)NC(Cc1ccccc1)C(=O)Nc1cccc2c1OCCO2 ; O=C(NC(Cc1ccccc1)C(=O)Nc1cccc2c1OCO2)c1ccccc1 ; COc1ccc(F)cc1NC(=O)C(Cc1ccccc1)NC(=O)c1ccccc1 ; O=C(NC(Cc1ccccc1)C(=O)Nc1cc(F)cc2ccccc12)c1ccccc1 ; Cc1ccc(F)cc1NC(=O)C(Cc1ccccc1)NC(=O)c1cccs1 ; Cc1ccc(F)cc1NC(=O)C(Cc1ccccc1)NC(=O)c1cccc2[nH]ccc12]

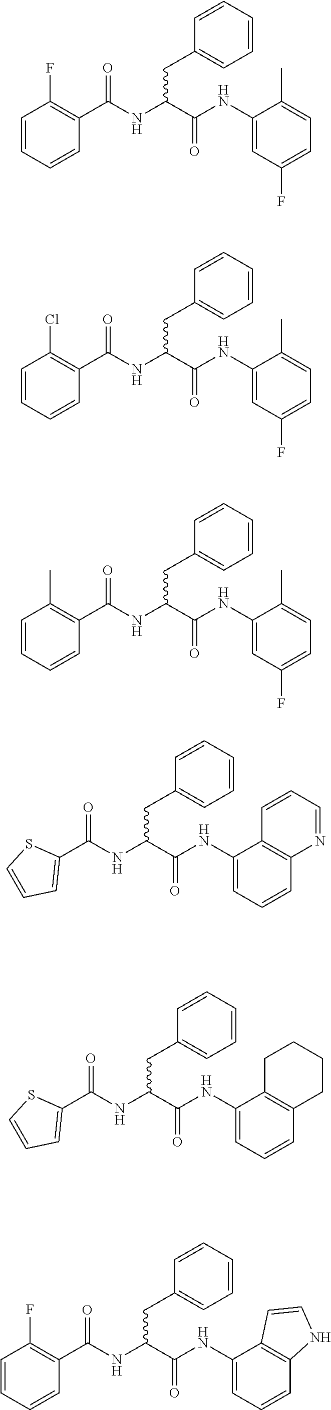 [SMILES: Cc1ccc(F)cc1NC(=O)C(Cc1ccccc1)NC(=O)c1ccccc1F ; Cc1ccc(F)cc1NC(=O)C(Cc1ccccc1)NC(=O)c1ccccc1Cl ; Cc1ccc(F)cc1NC(=O)C(Cc1ccccc1)NC(=O)c1ccccc1C ; O=C(NC(Cc1ccccc1)C(=O)Nc1cccc2ncccc12)c1cccs1 ; O=C(NC(Cc1ccccc1)C(=O)Nc1cccc2c1CCCC2)c1cccs1 ; O=C(NC(Cc1ccccc1)C(=O)Nc1cccc2[nH]ccc12)c1ccccc1F]

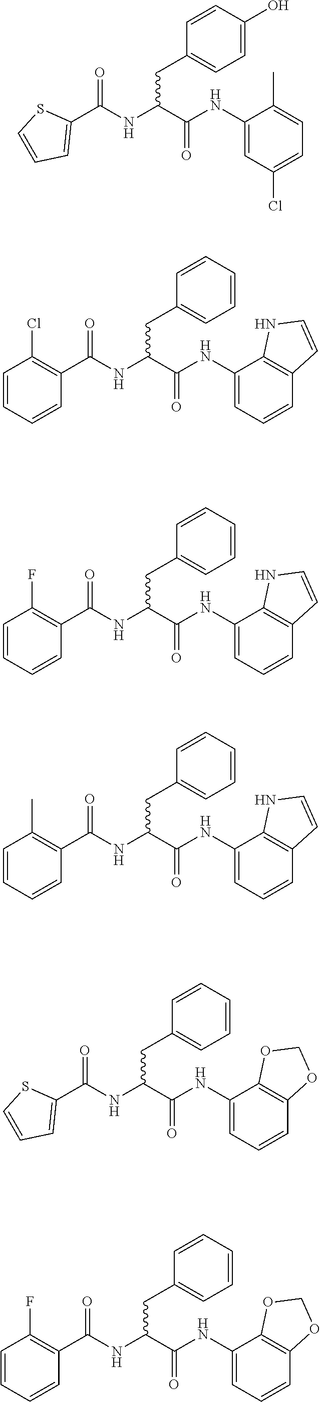 [SMILES: Cc1ccc(Cl)cc1NC(=O)C(Cc1ccc(O)cc1)NC(=O)c1cccs1 ; O=C(NC(Cc1ccccc1)C(=O)Nc1cccc2cc[nH]c12)c1ccccc1Cl ; O=C(NC(Cc1ccccc1)C(=O)Nc1cccc2cc[nH]c12)c1ccccc1F ; Cc1ccccc1C(=O)NC(Cc1ccccc1)C(=O)Nc1cccc2cc[nH]c12 ; O=C(NC(Cc1ccccc1)C(=O)Nc1cccc2c1OCO2)c1cccs1 ; O=C(NC(Cc1ccccc1)C(=O)Nc1cccc2c1OCO2)c1ccccc1F]

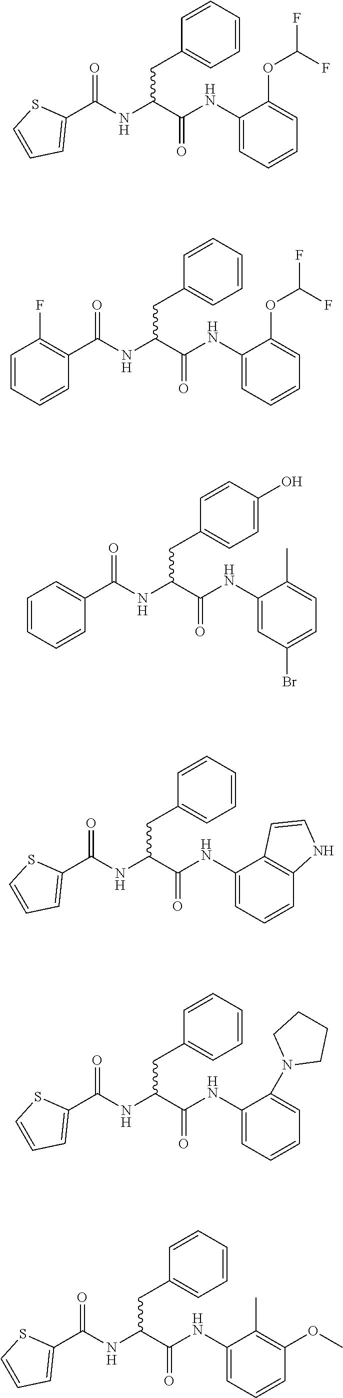 [SMILES: O=C(NC(Cc1ccccc1)C(=O)Nc1ccccc1OC(F)F)c1cccs1 ; O=C(NC(Cc1ccccc1)C(=O)Nc1ccccc1OC(F)F)c1ccccc1F ; Cc1ccc(Br)cc1NC(=O)C(Cc1ccc(O)cc1)NC(=O)c1ccccc1 ; O=C(NC(Cc1ccccc1)C(=O)Nc1cccc2[nH]ccc12)c1cccs1 ; O=C(NC(Cc1ccccc1)C(=O)Nc1ccccc1N1CCCC1)c1cccs1 ; COc1cccc(NC(=O)C(Cc2ccccc2)NC(=O)c2cccs2)c1C]

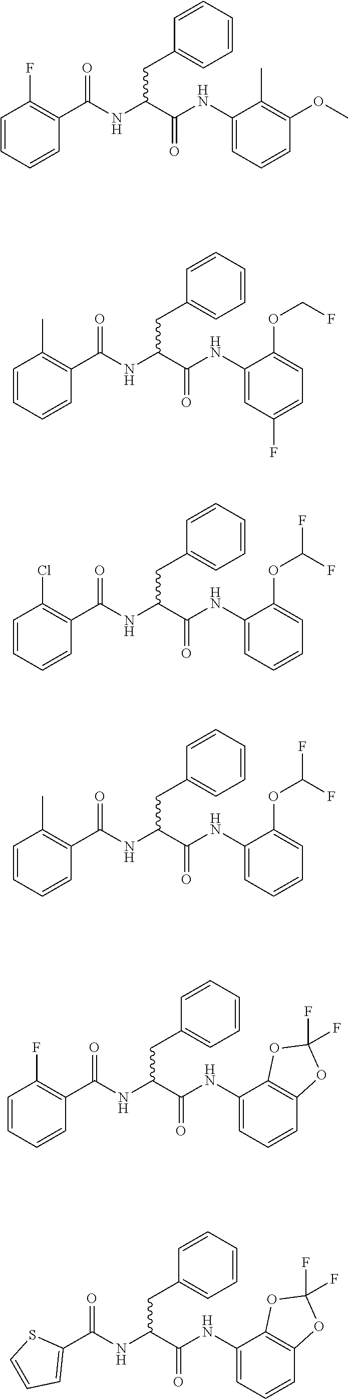 [SMILES: COc1cccc(NC(=O)C(Cc2ccccc2)NC(=O)c2ccccc2F)c1C ; Cc1ccccc1C(=O)NC(Cc1ccccc1)C(=O)Nc1cc(F)ccc1OCF ; O=C(NC(Cc1ccccc1)C(=O)Nc1ccccc1OC(F)F)c1ccccc1Cl ; Cc1ccccc1C(=O)NC(Cc1ccccc1)C(=O)Nc1ccccc1OC(F)F ; O=C(NC(Cc1ccccc1)C(=O)Nc1cccc2c1OC(F)(F)O2)c1ccccc1F ; O=C(NC(Cc1ccccc1)C(=O)Nc1cccc2c1OC(F)(F)O2)c1cccs1]

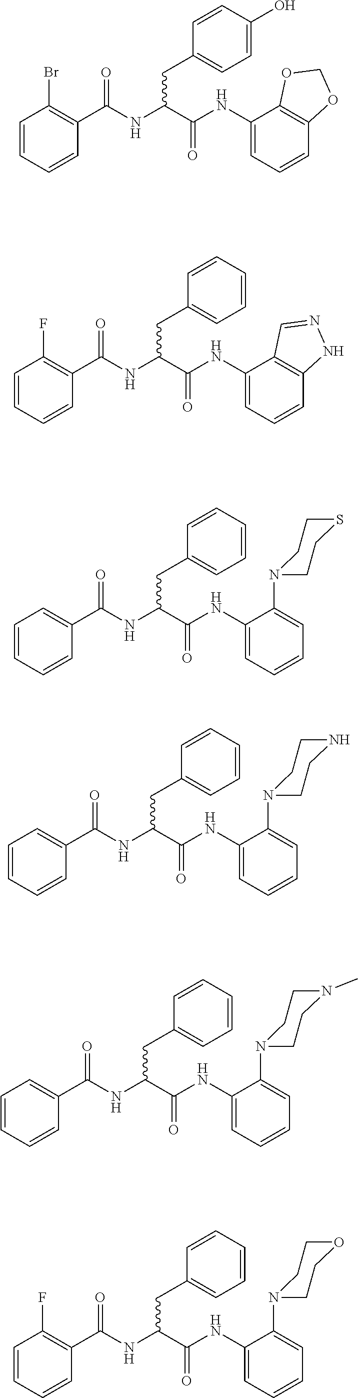 [SMILES: O=C(NC(Cc1ccc(O)cc1)C(=O)Nc1cccc2c1OCO2)c1ccccc1Br ; O=C(NC(Cc1ccccc1)C(=O)Nc1cccc2[nH]ncc12)c1ccccc1F ; O=C(NC(Cc1ccccc1)C(=O)Nc1ccccc1N1CCSCC1)c1ccccc1 ; O=C(NC(Cc1ccccc1)C(=O)Nc1ccccc1N1CCNCC1)c1ccccc1 ; CN1CCN(c2ccccc2NC(=O)C(Cc2ccccc2)NC(=O)c2ccccc2)CC1 ; O=C(NC(Cc1ccccc1)C(=O)Nc1ccccc1N1CCOCC1)c1ccccc1F]

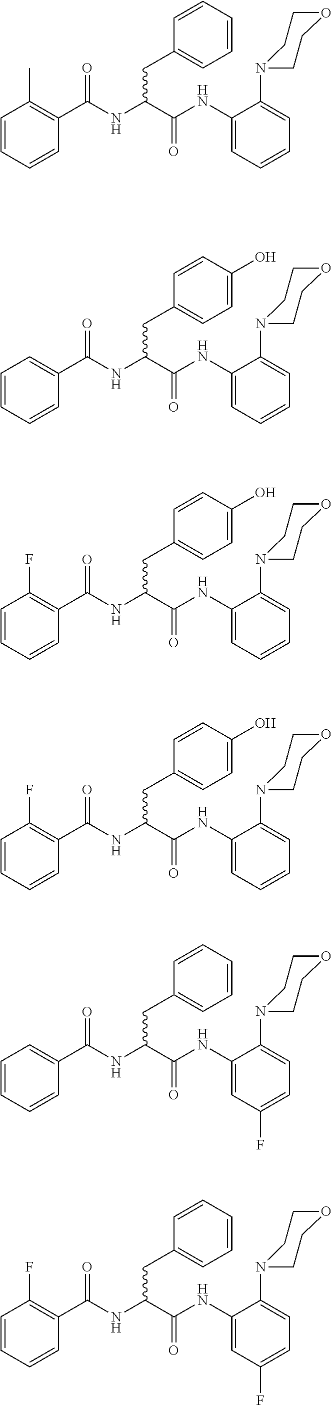 [SMILES: Cc1ccccc1C(=O)NC(Cc1ccccc1)C(=O)Nc1ccccc1N1CCOCC1 ; O=C(NC(Cc1ccc(O)cc1)C(=O)Nc1ccccc1N1CCOCC1)c1ccccc1 ; O=C(NC(Cc1ccc(O)cc1)C(=O)Nc1ccccc1N1CCOCC1)c1ccccc1F ; O=C(NC(Cc1ccc(O)cc1)C(=O)Nc1ccccc1N1CCOCC1)c1ccccc1F ; O=C(NC(Cc1ccccc1)C(=O)Nc1cc(F)ccc1N1CCOCC1)c1ccccc1 ; O=C(NC(Cc1ccccc1)C(=O)Nc1cc(F)ccc1N1CCOCC1)c1ccccc1F]

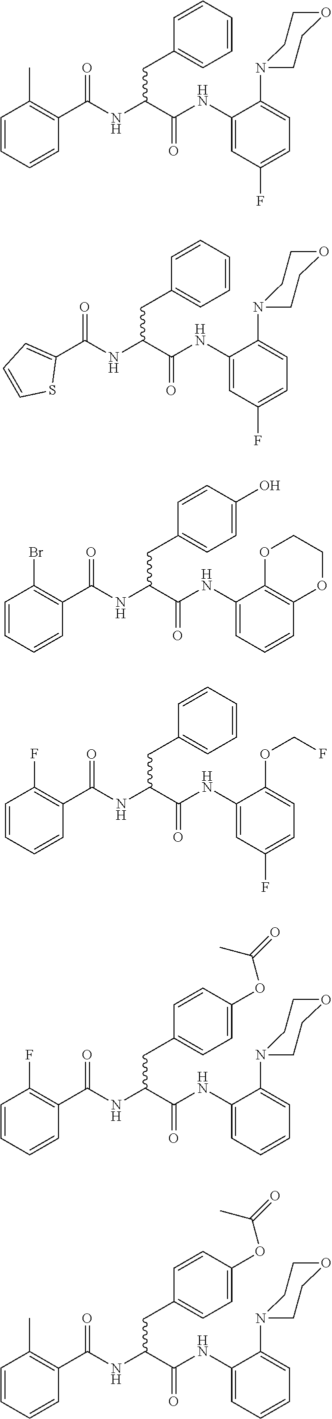

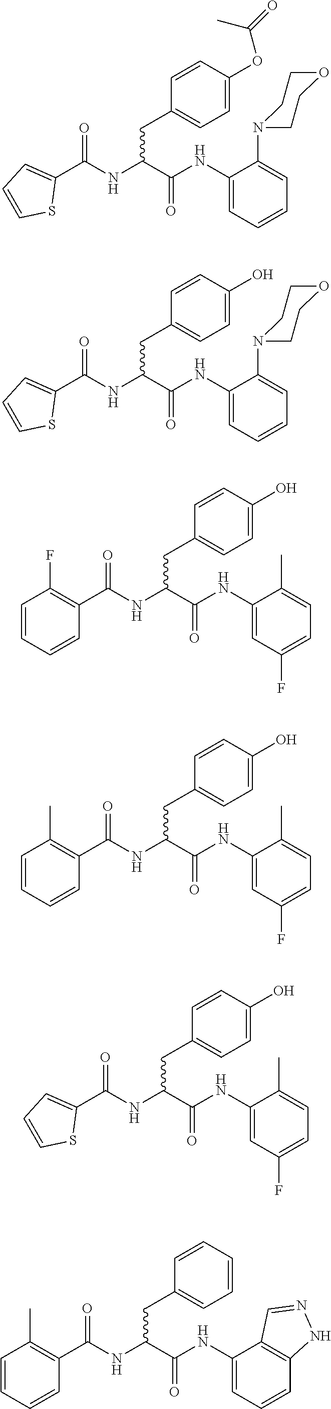 [SMILES: CC(=O)Oc1ccc(CC(NC(=O)c2cccs2)C(=O)Nc2ccccc2N2CCOCC2)cc1 ; O=C(NC(Cc1ccc(O)cc1)C(=O)Nc1ccccc1N1CCOCC1)c1cccs1 ; Cc1ccc(F)cc1NC(=O)C(Cc1ccc(O)cc1)NC(=O)c1ccccc1F ; Cc1ccc(F)cc1NC(=O)C(Cc1ccc(O)cc1)NC(=O)c1ccccc1C ; Cc1ccc(F)cc1NC(=O)C(Cc1ccc(O)cc1)NC(=O)c1cccs1 ; Cc1ccccc1C(=O)NC(Cc1ccccc1)C(=O)Nc1cccc2[nH]ncc12]

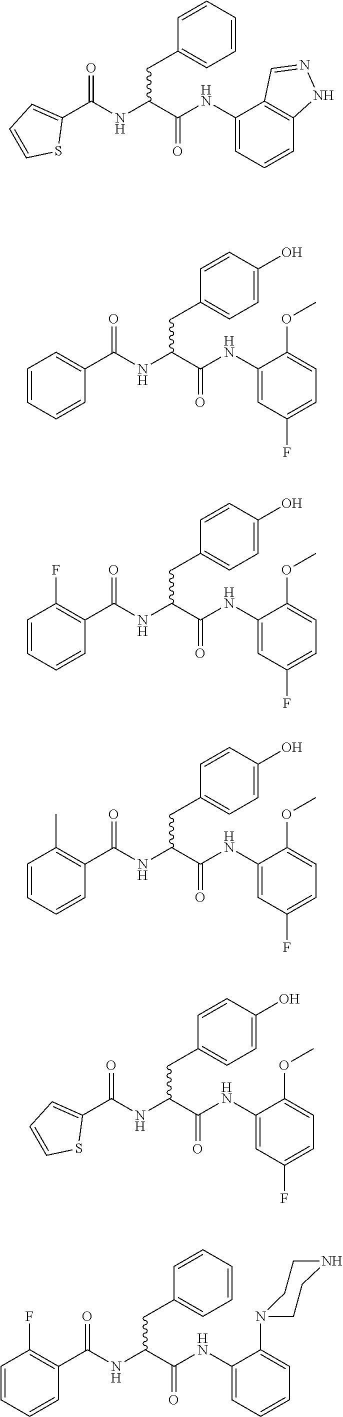

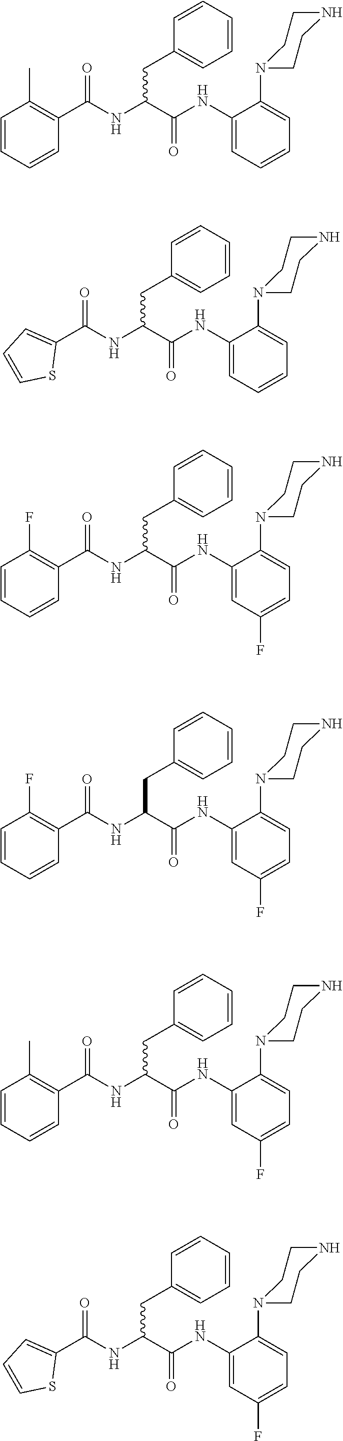 [SMILES: Cc1ccccc1C(=O)NC(Cc1ccccc1)C(=O)Nc1ccccc1N1CCNCC1 ; O=C(NC(Cc1ccccc1)C(=O)Nc1ccccc1N1CCNCC1)c1cccs1 ; O=C(NC(Cc1ccccc1)C(=O)Nc1cc(F)ccc1N1CCNCC1)c1ccccc1F ; O=C(N[C@@H](Cc1ccccc1)C(=O)Nc1cc(F)ccc1N1CCNCC1)c1ccccc1F ; Cc1ccccc1C(=O)NC(Cc1ccccc1)C(=O)Nc1cc(F)ccc1N1CCNCC1 ; O=C(NC(Cc1ccccc1)C(=O)Nc1cc(F)ccc1N1CCNCC1)c1cccs1]

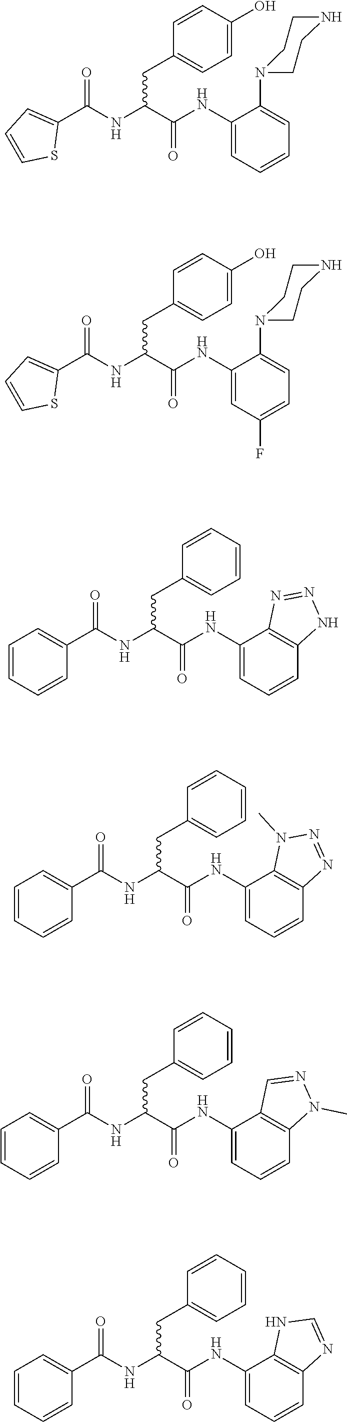 [SMILES: O=C(NC(Cc1ccc(O)cc1)C(=O)Nc1ccccc1N1CCNCC1)c1cccs1 ; O=C(NC(Cc1ccc(O)cc1)C(=O)Nc1cc(F)ccc1N1CCNCC1)c1cccs1 ; O=C(NC(Cc1ccccc1)C(=O)Nc1cccc2[nH]nnc12)c1ccccc1 ; Cn1nnc2cccc(NC(=O)C(Cc3ccccc3)NC(=O)c3ccccc3)c21 ; Cn1ncc2c(NC(=O)C(Cc3ccccc3)NC(=O)c3ccccc3)cccc21 ; O=C(NC(Cc1ccccc1)C(=O)Nc1cccc2nc[nH]c12)c1ccccc1]

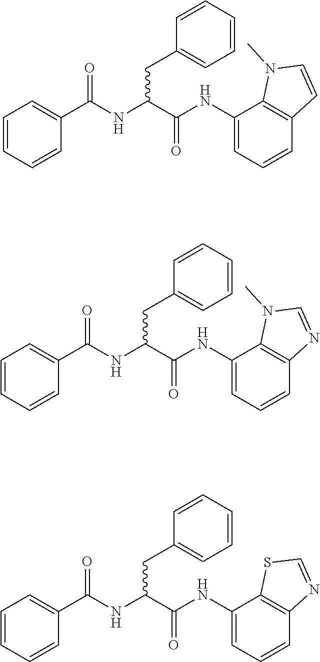 [SMILES: Cn1ccc2cccc(NC(=O)C(Cc3ccccc3)NC(=O)c3ccccc3)c21 ; Cn1cnc2cccc(NC(=O)C(Cc3ccccc3)NC(=O)c3ccccc3)c21 ; O=C(NC(Cc1ccccc1)C(=O)Nc1cccc2ncsc12)c1ccccc1]

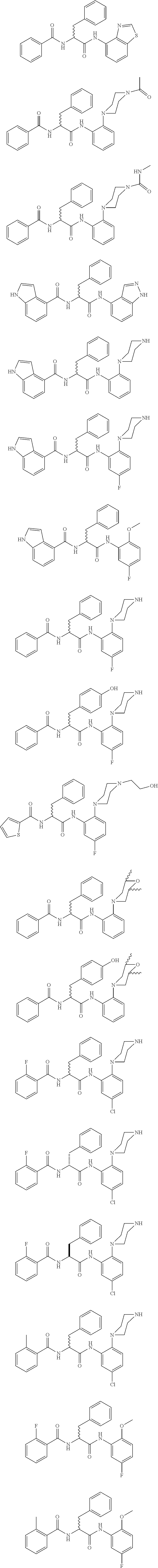

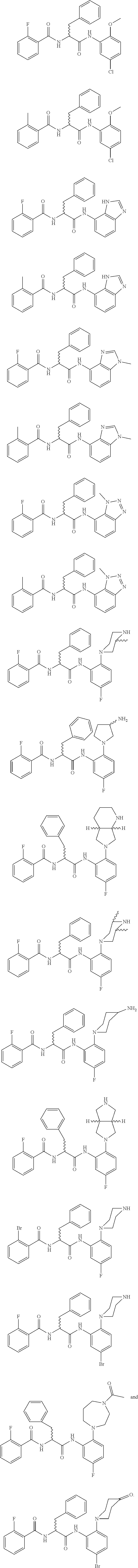

9. A compound, or a salt thereof, selected from: 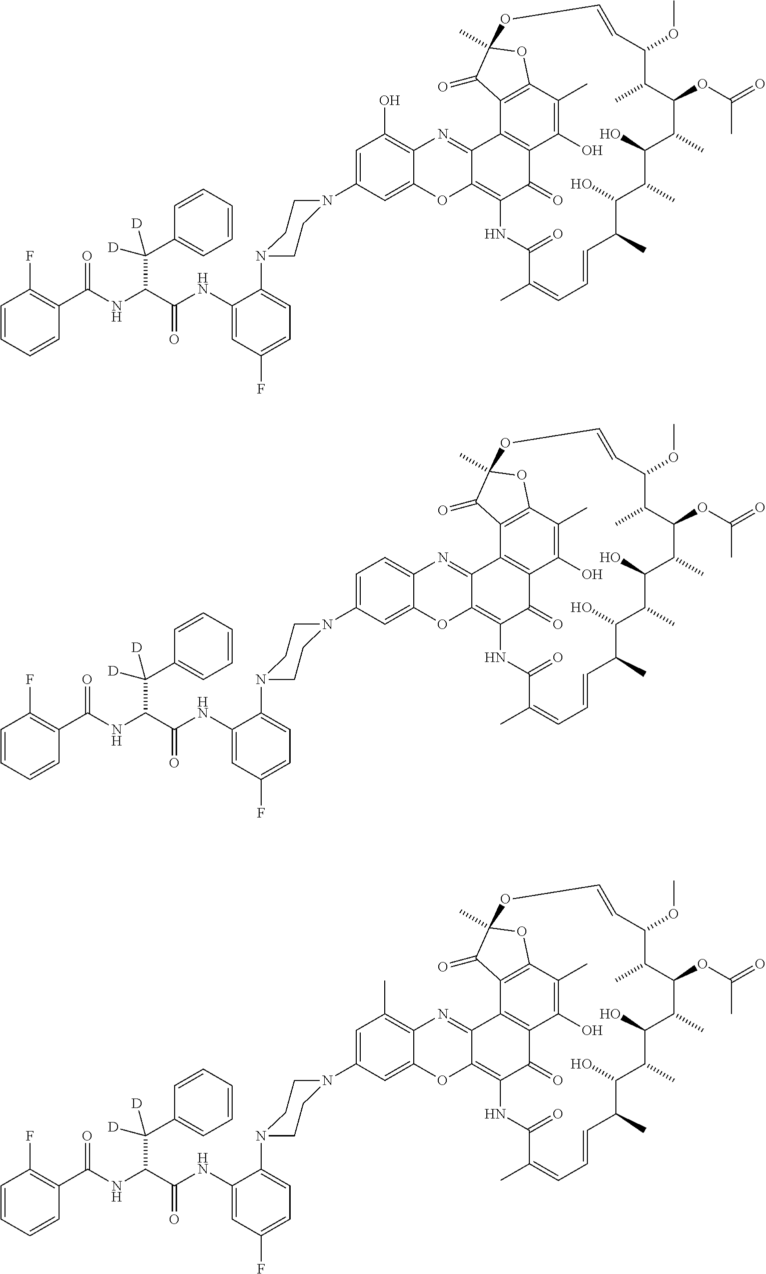 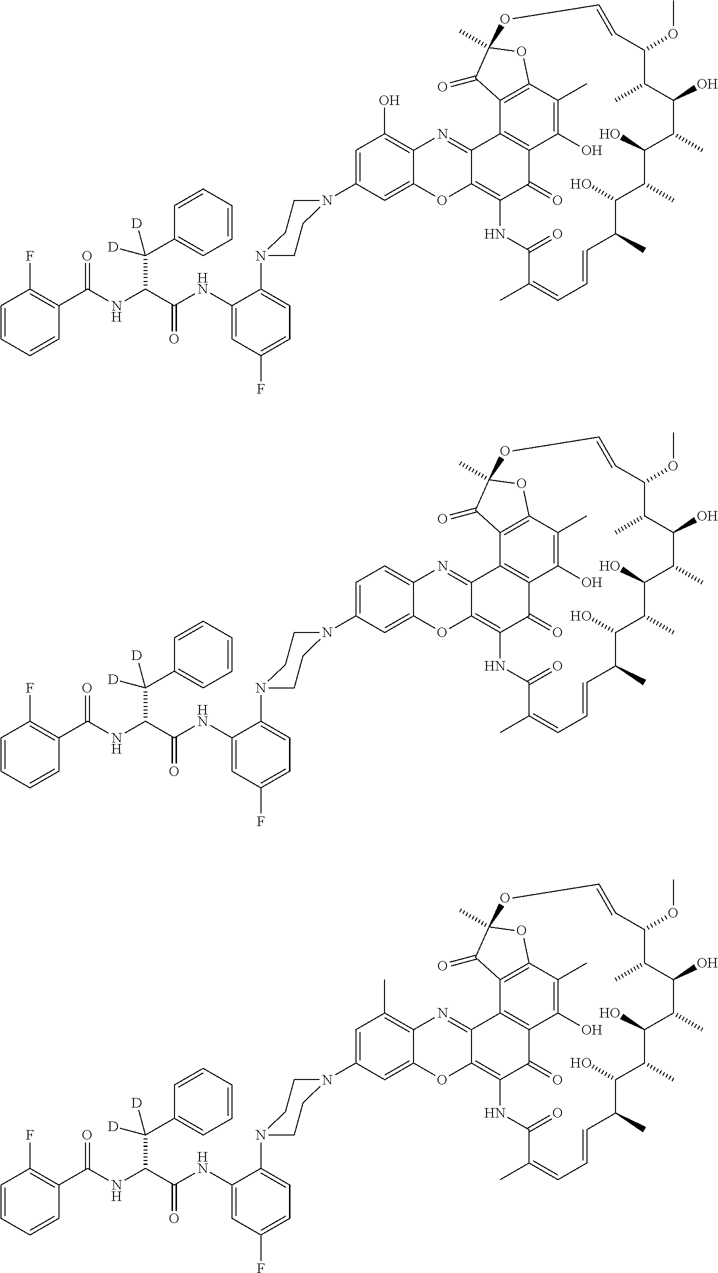 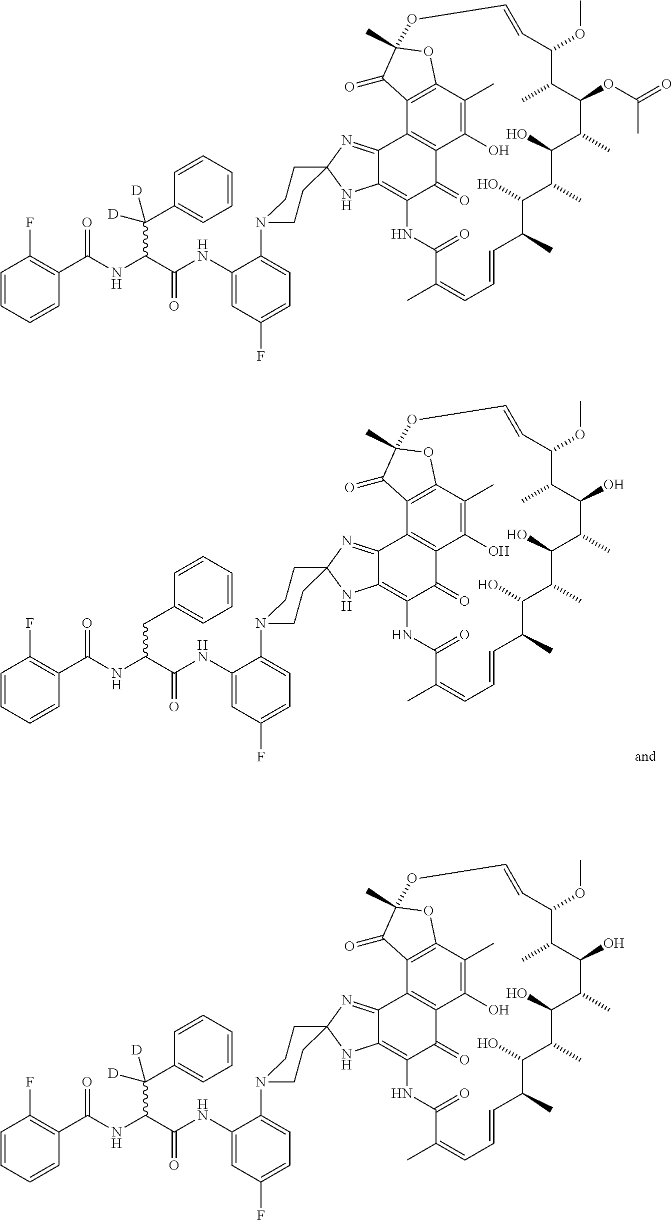
or a tautomer or salt thereof.

10. A method of making a compound as described in claim 1, wherein the compound is prepared from precursors α-β′ and ′β-γ, where β′ and ′β are moieties that can react to form β.

11. The method of claim 10, wherein precursors X-α′ and ′α-Y are allowed to react in the presence of a bacterial RNA polymerase.

12. The method of claim 11, wherein the bacterial RNA polymerase serves as a template for reaction of X-α′ and ′α-Y.

13. A method of making a compound as described herein.

14. Use of a compound, salt, or tautomer of any of claims 1-9 to inhibit an RNA polymerase from a bacterium.

15. Use of a compound, salt, or tautomer of any of claims 1-9 to inhibit an RNA polymerase from a Mycobacterium.

16. Use of a compound, salt, or tautomer of any of claims 1-9 to inhibit an RNA polymerase from one of Mycobacterium tuberculosis, Mycobacterium bovis, Mycobacterium avium, Mycobacterium abscessus, Mycobacterium chelonae, Mycobacterium fortuitum, Mycobacterium marimim, Mycobacterium leprae, Mycobacterium ulcerans, and Mycobacterium smegmatis.

17. Use of a compound, salt, or tautomer of any of claims 1-9 to inhibit one of the growth and the viability of a bacterium.

18. Use of a compound, salt, or tautomer of any of claims 1-9 to inhibit one of the growth and the viability of a Mycobacterium.

19. Use of a compound, salt, or tautomer of any of claims 1-9 to inhibit one of the growth and the viability of one of Mycobacterium tuberculosis, Mycobacterium bovis, Mycobacterium avium, Mycobacterium abscessus, Mycobacterium chelonae, Mycobacterium fortuitum, Mycobacterium marimim, Mycobacterium leprae, Mycobacterium ulcerans, and Mycobacterium smegmatis.

20. Use of a compound, salt, or tautomer of any of claims 1-8 to prevent or treat an infection by a bacterium.

21. Use of a compound, salt, or tautomer of any of claims 1-8 to prevent or treat an infection by a Mycobacterium.

22. Use of a compound, salt, or tautomer of any of claims 1-9 to prevent or treat an infection by one of Mycobacterium tuberculosis, Mycobacterium bovis, Mycobacterium avium, Mycobacterium abscessus, Mycobacterium chelonae, Mycobacterium fortuitum, Mycobacterium marinum, Mycobacterium leprae, Mycobacterium ulcerans, and Mycobacterium smegmatis.

23. A method of inhibiting a bacterial RNA polymerase, comprising contacting a bacterial RNA polymerase with a compound, salt, or tautomer of any of claims 1-9.

24. A method of inhibiting one of the growth and the viability of a bacterium, comprising contacting a bacterium with a compound, salt, or tautomer of any of claims 1-9.

25. A method of preventing a bacterial infection, comprising administering to a mammal a compound, salt, or tautomer of any of claims 1-9.

26. A method of treating a bacterial infection, comprising administering to a mammal a compound, salt, or tautomer of any of claims 1-9.

27. A formulation comprising a compound, salt, or tautomer of any of claims 1-9, for administration to a mammal to prevent a bacterial infection.

28. A formulation comprising a compound, salt, or tautomer of any of claims 1-9, for administration to a mammal to treat a bacterial infection.

29. Administration of a formulation comprising a compound, salt, or tautomer of any of claims 1-9.

30. Administration of a formulation comprising a compound, salt, or tautomer of any of claims 1-8 to a mammal to prevent or treat a bacterial infection.

31. Use of a compound, salt, or tautomer of any of claims 1-9 to bind to a bacterial RNA polymerase.

32. Use of a compound, salt, or tautomer of any of claims 1-9 to inhibit a bacterial RNA polymerase.

33. Use of a compound, salt, or tautomer of any of claims 1-9 to inhibit bacterial gene expression.

34. Use of a compound, salt, or tautomer of any of claims 1-9 to inhibit bacterial growth.

35. Use of a compound, salt, or tautomer of any of claims 1-9 to treat a bacterial infection.

36. A composition comprising a compound or tautomer of any of claims 1-9 or a pharmaceutically acceptable salt thereof, and a pharmaceutically acceptable vehicle.

37. A method for inhibiting a bacterial RNA polymerase comprising contacting the bacterial RNA polymerase with a compound, salt, or tautomer of any of claims 1-9.

38. A method for inhibiting the growth of bacteria comprising contacting the bacteria with a compound, salt, or tautomer of any of claims 1-9.

39. A method for treating a bacterial infection in a mammal comprising administering to the mammal an effective amount of a compound or tautomer of any of claims 1-9 or a pharmaceutically acceptable salt thereof.

40. A compound or tautomer of any of claims 1-9, or a pharmaceutically acceptable salt thereof, for use in the prophylactic or therapeutic treatment of a bacterial infection.

41. The use of compound or tautomer of any of claims 1-9, or a pharmaceutically acceptable salt thereof, for the preparation of a medicament for treating a bacterial infection in a mammal.

42. A compound or tautomer of any of claims 1-9, or a pharmaceutically acceptable salt thereof for use in medical treatment.
